SEQUENCE LISTING (47 sequences)

SEQ ID NO: 1 - protein, 10 aa, Artificial Sequence
  Description of Artificial Sequence Synthetic peptide
GYTFTSYWIH

SEQ ID NO: 2 - protein, 17 aa, Artificial Sequence
  Description of Artificial Sequence Synthetic peptide
LIDPSDSYTNYNQKFRG

SEQ ID NO: 3 - protein, 6 aa, Artificial Sequence
  Description of Artificial Sequence Synthetic peptide
  MOD_RES (6)..(6): Any amino acid
SDPTGX

SEQ ID NO: 4 - protein, 16 aa, Artificial Sequence
  Description of Artificial Sequence Synthetic peptide
RSSKSLLHSSGITYLS

SEQ ID NO: 5 - protein, 7 aa, Artificial Sequence
  Description of Artificial Sequence Synthetic peptide
QMSNLAS

SEQ ID NO: 6 - protein, 9 aa, Artificial Sequence
  Description of Artificial Sequence Synthetic peptide
AQNLELPLT

SEQ ID NO: 7 - protein, 115 aa, Artificial Sequence
  Description of Artificial Sequence Synthetic polypeptide
QVQLVQSGAEVKKPGSSVKVSCKASGYTFTSYWIHWVRQAPGQGLEWIGLIDPSDSYTNYNQKFRGRVTITVDTSTSTAYMELSSLRSEDTAVYYCASSDPTGCWGQGTTVTVSS

SEQ ID NO: 8 - protein, 115 aa, Artificial Sequence
  Description of Artificial Sequence Synthetic polypeptide
QVQLVQSGAEVKKPGSSVKVSCKASGYTFTSYWIHWVRQAPGQGLEWIGLIDPSDSYTNYNQKFRGRVTITVDTSTSTAYMELSSLRSEDTAVYYCASSDPTGAWGQGTTVTVSS

SEQ ID NO: 9 - protein, 115 aa, Artificial Sequence
  Description of Artificial Sequence Synthetic polypeptide
QVQLVQSGAEVKKPGSSVKVSCKASGYTFTSYWIHWVRQAPGQGLEWIGLIDPSDSYTNYNQKFRGRVTITVDTSTSTAYMELSSLRSEDTAVYYCASSDPTGGWGQGTTVTVSS

SEQ ID NO: 10 - protein, 115 aa, Artificial Sequence
  Description of Artificial Sequence Synthetic polypeptide
QVQLVQSGAEVKKPGSSVKVSCKASGYTFTSYWIHWVRQAPGQGLEWIGLIDPSDSYTNYNQKFRGRVTITVDTSTSTAYMELSSLRSEDTAVYYCASSDPTGLWGQGTTVTVSS

SEQ ID NO: 11 - protein, 115 aa, Artificial Sequence
  Description of Artificial Sequence Synthetic polypeptide
QVQLVQSGAEVKKPGSSVKVSCKASGYTFTSYWIHWVRQAPGQGLEWIGLIDPSDSYTNYNQKFRGRVTITVDTSTSTAYMELSSLRSEDTAVYYCASSDPTGSWGQGTTVTVSS

SEQ ID NO: 12 - protein, 115 aa, Artificial Sequence
  Description of Artificial Sequence Synthetic polypeptide
QVQLVQSGAEVKKPGSSVKVSCKASGYTFTSYWIHWVRQAPGQGLEWIGLIDPSDSYTNYNQKFRGRVTITVDTSTSTAYMELSSLRSEDTAVYYCASSDPTGTWGQGTTVTVSS

SEQ ID NO: 13 - protein, 115 aa, Artificial Sequence
  Description of Artificial Sequence Synthetic polypeptide
QVQLVQSGAEVKKPGSSVKVSCKASGYTFTSYWIHWVRQAPGQGLEWIGLIDPSDSYTNYNQKFRGRVTITVDTSTSTAYMELSSLRSEDTAVYYCASSDPTGVWGQGTTVTVSS

SEQ ID NO: 14 - protein, 115 aa, Artificial Sequence
  Description of Artificial Sequence Synthetic polypeptide
QVQLVQSGAEVKKPGSSVKVSCKASGYTFTSYWIHWVRQAPGQGLEWIGLIDPSDSYTNYNQKFRGRVTITRDTSTSTAYMELSSLRSEDTAVYYCASSDPTGCWGQGTTVTVSS

SEQ ID NO: 15 - protein, 115 aa, Artificial Sequence
  Description of Artificial Sequence Synthetic polypeptide
QVQLVQSGAEVKKPGSSVKVSCKASGYTFTSYWIHWVRQAPGQGLEWIGLIDPSDSYTNYNQKFRGRVTITRDTSTSTAYMELSSLRSEDTAVYYCASSDPTGAWGQGTTVTVSS

SEQ ID NO: 16 - protein, 115 aa, Artificial Sequence
  Description of Artificial Sequence Synthetic polypeptide
QVQLVQSGAEVKKPGSSVKVSCKASGYTFTSYWIHWVRQAPGQGLEWIGLIDPSDSYTNYNQKFRGRVTITRDTSTSTAYMELSSLRSEDTAVYYCASSDPTGGWGQGTTVTVSS

SEQ ID NO: 17 - protein, 115 aa, Artificial Sequence
  Description of Artificial Sequence Synthetic polypeptide
QVQLVQSGAEVKKPGSSVKVSCKASGYTFTSYWIHWVRQAPGQGLEWIGLIDPSDSYTNYNQKFRGRVTITRDTSTSTAYMELSSLRSEDTAVYYCASSDPTGLWGQGTTVTVSS

SEQ ID NO: 18 - protein, 115 aa, Artificial Sequence
  Description of Artificial Sequence Synthetic polypeptide
QVQLVQSGAEVKKPGSSVKVSCKASGYTFTSYWIHWVRQAPGQGLEWIGLIDPSDSYTNYNQKFRGRVTITRDTSTSTAYMELSSLRSEDTAVYYCASSDPTGSWGQGTTVTVSS

SEQ ID NO: 19 - protein, 115 aa, Artificial Sequence
  Description of Artificial Sequence Synthetic polypeptide
QVQLVQSGAEVKKPGSSVKVSCKASGYTFTSYWIHWVRQAPGQGLEWIGLIDPSDSYTNYNQKFRGRVTITRDTSTSTAYMELSSLRSEDTAVYYCASSDPTGYWGQGTTVTVSS

SEQ ID NO: 20 - protein, 115 aa, Artificial Sequence
  Description of Artificial Sequence Synthetic polypeptide
QVQLVQSGAEVKKPGSSVKVSCKASGYTFTSYWIHWVRQAPGQGLEWIGLIDPSDSYTNYNQKFRGRVTITRDTSTSTAYMELSSLRSEDTAVYYCASSDPTGVWGQGTTVTVSS

SEQ ID NO: 21 - protein, 112 aa, Artificial Sequence
  Description of Artificial Sequence Synthetic polypeptide
DIVMTQAAFSNPVTPGTPASISCRSSKSLLHSSGITYLSWYLQKPGQSPQLLIYQMSNLASGVPDRFSSSGSGTDFTLKISRVEAEDVGVYYCAQNLELPLTFGQGTKLEIK

SEQ ID NO: 22 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 107 aa, Homo sapiens [sequence omitted]

SEQ ID NO: 24 - protein, 6 aa, Artificial Sequence
  Description of Artificial Sequence Synthetic peptide
SDPTGC

SEQ ID NO: 25 - protein, 6 aa, Artificial Sequence
  Description of Artificial Sequence Synthetic peptide
SDPTGA

SEQ ID NO: 26 - protein, 6 aa, Artificial Sequence
  Description of Artificial Sequence Synthetic peptide
SDPTGG

SEQ ID NO: 27 - protein, 6 aa, Artificial Sequence
  Description of Artificial Sequence Synthetic peptide
SDPTGL

SEQ ID NO: 28 - protein, 6 aa, Artificial Sequence
  Description of Artificial Sequence Synthetic peptide
SDPTGS

SEQ ID NO: 29 - protein, 6 aa, Artificial Sequence
  Description of Artificial Sequence Synthetic peptide
SDPTGT

SEQ ID NO: 30 - protein, 6 aa, Artificial Sequence
  Description of Artificial Sequence Synthetic peptide
SDPTGV

SEQ ID NO: 31 - protein, 19 aa, Homo sapiens
YSMKKTTMKIIPFNRLTIG

SEQ ID NO: 32 - DNA, 345 bp, Artificial Sequence
  Description of Artificial Sequence Synthetic polynucleotide
caagtgcagctggtgcagtcgggagccgaagtcaagaagcctggttcaagcgtgaaagtgtcctgcaaggcctccgggtacaccttcacgtcctactggattcactgggtccgccaagcaccaggacagggcctggagtggatcggactgatcgaccccagcgactcctacactaactacaaccagaagttccggggcagagtgaccatcaccgtggatacctcgaccagcactgcttatatggaactgtcgtccctccggtccgaggacactgccgtgtactactgcgcgtcatccgatccgactgggtgttggggccagggaaccaccgtgacagtctcctca

SEQ ID NO: 33 - DNA, 345 bp, Artificial Sequence
  Description of Artificial Sequence Synthetic polynucleotide
caagtgcagctcgtgcagtccggagccgaagtcaagaagcctggcagctcagtgaaagtgtcctgcaaggcctcgggatacaccttcacttcctactggattcactgggtcagacaagcgccaggacagggactggagtggatcgggctgatcgatccgtccgactcctacaccaactataaccagaagttcagaggccgcgtgaccatcaccagggacacttccacctcgacggcttacatggagcttagctccctgcggtcggaagataccgccgtgtactactgcgcaagctcagaccccaccggttgttggggccagggcactactgtgacagtctcctca

SEQ ID NO: 34 - DNA, 345 bp, Artificial Sequence
  Description of Artificial Sequence Synthetic polynucleotide
caagtgcagcttcagcagccgggtgctgaactggtccgccctggcacttccgtgaagctcagctgtaaagcctccggctacaccttcacctcatactggattcactgggtcaagcaacggccaggacaagggttggaatggatcgggctcatcgatccgtccgattcgtacactaactataaccagaagttcagaggaaaggccaccctgaccgtggacacctcgtcgtccaccgcgtacatgcagctgtcaagcctgacttccgaggactccgccgtgtactactgcgcaagctccgaccccactggatgctggggccagggtaccacgctgacagtctcctca

SEQ ID NO: 35 - protein, 115 aa, Artificial Sequence
  Description of Artificial Sequence Synthetic polypeptide
QVQLQQPGAELVRPGTSVKLSCKASGYTFTSYWIHWVKQRPGQGLEWIGLIDPSDSYTNYNQKFRGKATLTVDTSSSTAYMQLSSLTSEDSAVYYCASSDPTGCWGQGTTLTVSS

SEQ ID NO: 36 - protein, 158 aa, Mus sp.
TFDSPYQVRRMRFSAQLLGLLVLWIPGSTADIVMTQAAFSNPITLGTSASMSCRSSKSLLHSSGITYLSWYLQKPGQSPQLLIYQMSNLASGVPDRFSSSGSGTDFTLRISRVEAEDVGVYYCAQNLELPLTFGAGTKLELKRADAAPTVSACTKGEF

SEQ ID NO: 37 - protein, 166 aa, Mus sp.
NTAFAGFGRNMRSLFSLQLLSTQDLAMGWSCIIVLLVSTATGVHSQVQLQQPGAELVRPGTSVKLSCKASGYTFTSYWIHWVKQRPGQGLEWIGLIDPSDSYTNYNQKFRGKATLTVDTSSSTAYMQLSSLTSEDSAVYYCASSDPTGCWGQGTTLTVSPASTTPP

SEQ ID NO: 38 - protein, 113 aa, Artificial Sequence
  Description of Artificial Sequence Synthetic polypeptide
QVQLQQPGAELVRPGTSVKLSCKASGYTFTSYWHWVKQRPGQGLEWGLIDPSDSYTNYNQKFRGKATLTVDTSSSTAYMQLSSLTSEDSAVYYCASSDPTGCWGQGTTLTVSS

SEQ ID NO: 39 - protein, 113 aa, Artificial Sequence
  Description of Artificial Sequence Synthetic polypeptide
QVQLVQSGAELKKPGTSVKLSCKASGYTFTSYWHWVKQAPGQGLEWGLIDPSDSYTNYNQKFRGRATLTVDTSTSTAYMQLSSLTSEDSAVYYCASSDPTGCWGQGTTVTVSS

SEQ ID NO: 40 - protein, 113 aa, Artificial Sequence
  Description of Artificial Sequence Synthetic polypeptide
QVQLVQSGAEVKKPGSSVKLSCKASGYTFTSYWHWVKQAPGQGLEWGLIDPSDSYTNYNQKFRGRATLTVDTSTSTAYMELSSLRSEDTAVYYCASSDPTGCWGQGTTVTVSS

SEQ ID NO: 41 - protein, 113 aa, Artificial Sequence
  Description of Artificial Sequence Synthetic polypeptide
QVQLVQSGAEVKKPGSSVKVSCKASGYTFTSYWHWVRQAPGQGLEWGLIDPSDSYTNYNQKFRGRVTITVDTSTSTAYMELSSLRSEDTAVYYCASSDPTGCWGQGTTVTVSS

SEQ ID NO: 42 - protein, 113 aa, Artificial Sequence
  Description of Artificial Sequence Synthetic polypeptide
QVQLVQSGAEVKKPGSSVKVSCKASGYTFTSYWHWVRQAPGQGLEWGLIDPSDSYTNYNQKFRGRVTITRDTSTSTAYMELSSLRSEDTAVYYCASSDPTGCWGQGTTVTVSS

SEQ ID NO: 43 - protein, 113 aa, Artificial Sequence
  Description of Artificial Sequence Synthetic polypeptide
QVQLVQSGAEVKKPGSSVKVSCKASGYTFTSYWHWVRQAPGQGLEWGLIDPSDSYTNYNQKFQGRVTITRDTSTSTAYMELSSLRSEDTAVYYCASSDPTGCWGQGTTVTVSS

SEQ ID NO: 44 - protein, 112 aa, Artificial Sequence
  Description of Artificial Sequence Synthetic polypeptide
DIVMTQAAFSNPITLGTSASMSCRSSKSLLHSSGITYLSWYLQKPGQSPQLLIYQMSNLASGVPDRFSSSGSGTDFTLRISRVEAEDVGVYYCAQNLELPLTFGAGTKLELK

SEQ ID NO: 45 - protein, 112 aa, Artificial Sequence
  Description of Artificial Sequence Synthetic polypeptide
DIVMTQAAFSNPITPGTPASISCRSSKSLLHSSGITYLSWYLQKPGQSPQLLIYQMSNLASGVPDRFSSSGSGTDFTLRISRVEAEDVGVYYCAQNLELPLTFGQGTKLEIK

SEQ ID NO: 46 - protein, 112 aa, Artificial Sequence
  Description of Artificial Sequence Synthetic polypeptide
DIVMTQSPLSLPVTPGEPASISCRSSKSLLHSSGITYLSWYLQKPGQSPQLLIYQMSNLASGVPDRFSSSGSGTDFTLKISRVEAEDVGVYYCAQNLELPLTFGQGTKLEIK

SEQ ID NO: 47 - protein, 112 aa, Artificial Sequence
  Description of Artificial Sequence Synthetic polypeptide
DIVMTQSPLSLPVTPGEPASISCRSSKSLLHSSGITYLSWYLQKPGQSPQLLIYQMSNLASGVPDRFSGSGSGTDFTLKISRVEAEDVGVYYCAQNLELPLTFGQGTKLEIK

(19) United States
(12) Patent Application Publication
(10) Pub. No.: US 2024/0279357 A1
(43) Pub. Date: Aug. 22, 2024

(54) ANTIBODIES WHICH BIND HUMAN FIBRIN OR FIBRINOGEN yC DOMAIN AND METHODS OF USE

(71) Applicant: Therini Bio, Inc., South San Francisco, CA (US)

(72) Inventors: Jeffrey Stavenhagen, South San Francisco, CA (US); Katerina Akassoglou, South San Francisco, CA (US)

(21) Appl. No.: 18/571,091

(22) PCT Filed: Jun. 20, 2022
(86) PCT No.: PCT/US2022/034188

§ 371 Date: Dec. 15, 2023

Related U.S. Application Data
(60) Provisional application No. 63/212,409, filed on Jun. 18, 2021.

(51) Int. Cl. C07K 16/36; A61K 39/00; A61P 1/00; A61P 25/00; C07K 16/18

(52) U.S. Cl. CPC C07K 16/36; A61P 1/00; A61P 25/00; C07K 16/18; A61K 2039/505; C07K 2317/24; C07K 2317/565; C07K 2317/76; C07K 2317/92; C07K 2317/94

(57) ABSTRACT

Described herein are novel and improved antibodies that bind human fibrin or fibrinogen γC domain and methods of use thereof. In certain aspects, described herein are methods of inhibiting microglial activation. In certain aspects, described herein are pharmaceutical compositions comprising the antibodies that bind fibrin or fibrinogen γC domain. In certain aspects, the antibodies and methods described herein are used for treatment of degenerative neuronal disorders that involve inflammatory demyelination.

### CROSS REFERENCE TO RELATED APPLICATIONS

This application claims the benefit of U.S. Provisional Application No. 63/212,409 filed Jun. 18, 2021, which is hereby incorporated in its entirety by reference.

### SEQUENCE LISTING

Not applicable.

### BACKGROUND

Degenerative neuronal disorders such as multiple sclerosis (MS) can involve inflammatory demyelination and autoimmune responses. Microglia, in particular perivascular microglia, are believed to be necessary not only for the maintenance, but also for the onset of inflammatory demyelination in central nervous system (CNS) autoimmune disease. Activation of microglia contributes to both neuronal and oligodendrocyte death via release of cytokines and nitric oxide. In MS, inflammatory processes are associated with destruction of myelin sheaths, and can also involve axonal damage that can lead to permanent functional deficits, such as paralysis and loss of vision. Resident microglia are responsible for demyelination, via their ability to phagocytose myelin and secrete proinflammatory cytokines.

In MS lesions, perivascular activation of microglia colocalizes with areas of blood brain barrier (BBB) disruption, and in vivo imaging studies have shown that BBB disruption provokes the immediate and focal activation of microglia. One of the earliest events coupled to BBB disruption in MS is leakage of the blood protein fibrinogen in the nervous system that results in perivascular deposition of fibrin. Fibrinogen is not present in the healthy CNS, but only leaks in the brain after BBB disruption, thus serving as an environmental “danger” signal. Upon conversion of fibrinogen to fibrin, the CD11b/CD18 integrin receptor (also referred to as: Mac-1, aMfl 2, Complement Receptor 3) binds to the fibrin and induces microglial activation leading to inflammatory demyelination. CD11b is the alpha chain of the receptor that regulates phagocytosis of myelin during inflammatory demyelination. Immobilized fibrinogen and insoluble fibrin, but not soluble fibrinogen, have been identified as physiological, high-affinity ligands for Mac-1.

The γ377-395 epitope of the fibrin or fibrinogen γC domain is the binding epitope of fibrin to CDIIb. The fibrin γ377-395 peptide functions as an inhibitor of microglia activation by blocking fibrin binding to Mac-1. Because fibrin mediates blood coagulation by binding via a distinct epitope to the platelet integrin a_{IIb}β₃ receptor, therapeutic agents (including antibodies), that block CD11b binding epitope to fibrin can reduce the damaging effects of fibrin in the nervous system without affecting its beneficial effects in blood coagulation. Therefore, safe, effective antibodies that inhibit fibrin induced microglial activation without affecting its beneficial effects in blood coagulation are needed as therapeutics for degenerative neuronal disorders that involve inflammatory demyelination.

### SUMMARY

In an aspect, described herein is an isolated antibody that binds human fibrin or fibrinogen γC domain, comprising a heavy chain comprising a variable heavy (VH) chain sequence comprising three heavy chain CDR sequences, CDR-H1, CDR-H2, and CDR-H3, and a light chain comprising a variable light (VL) chain sequence comprising three light chain CDR sequences, CDR-L1, CDR-L2, and CDR-L3, wherein: i) CDR-H1 comprises the sequence set forth in SEQ ID NO: 1; ii) CDR-H2 comprises the sequence set forth in SEQ ID NO: 2; iii) CDR-H3 comprises the sequence set forth in SEQ ID NO: 3, wherein X is Glycine (G), Valine (V), Threonine (T), Serine (S), Alanine (A) or Leucine (L); iv) CDR-L1 comprises the sequence set forth in SEQ ID NO: 4; v) CDR-L2 comprises the sequence set forth in SEQ ID NO: 5, and vi) CDR-L3 comprises the sequence set forth in SEQ ID NO: 6.

In an embodiment, the isolated antibody comprises a VH sequence selected from a sequence set forth in one of SEQ ID NOs: 7-20. In an embodiment, the isolated antibody comprises a VL sequence selected from a sequence set forth in SEQ ID NO 21. In an embodiment, the isolated antibody comprises a VH sequence selected from a sequence set forth in one of SEQ ID Nos: 7-20, and the VL sequence set for in SEQ ID NO: 21. In an embodiment, the isolated antibody comprises a VH sequence set forth in SEQ ID NO: 7 and a VL sequence set forth in SEQ ID NO: 21. In an embodiment, the isolated antibody comprises a VH sequence set forth in SEQ ID NO: 8 and a VL sequence set forth in SEQ ID NO: 21. In an embodiment, the isolated antibody comprises a VH sequence set forth in SEQ ID NO: 9 and a VL sequence set forth in SEQ ID NO: 21. In an embodiment, the isolated antibody comprises a VH sequence set forth in SEQ ID NO: 10 and a VL sequence set forth in SEQ ID NO: 21. In an embodiment, the isolated antibody comprises a VH sequence set forth in SEQ ID NO: 11 and a VL sequence set forth in SEQ ID NO: 21. In an embodiment, the isolated antibody comprises a VH sequence set forth in SEQ ID NO: 12 and a VL sequence set forth in SEQ ID NO: 21. In an embodiment, the isolated antibody comprises a VH sequence set forth in SEQ ID NO: 13 and a VL sequence set forth in SEQ ID NO: 21. In an embodiment, the isolated antibody comprises a VH sequence set forth in SEQ ID NO: 14 and a VL sequence set forth in SEQ ID NO: 21. In an embodiment, the isolated antibody comprises a VH sequence set forth in SEQ ID NO: 15 and a VL sequence set forth in SEQ ID NO: 21. In an embodiment, the isolated antibody comprises a VH sequence set forth in SEQ ID NO: 16 and a VL sequence set forth in SEQ ID NO: 21. In an embodiment, the isolated antibody comprises a VH sequence set forth in SEQ ID NO: 17 and a VL sequence set forth in SEQ ID NO: 21. In an embodiment, the isolated antibody comprises a VH sequence set forth in SEQ ID NO: 18 and a VL sequence set forth in SEQ ID NO: 21. In an embodiment, the isolated antibody comprises a VH sequence set forth in SEQ ID NO: 19 and a VL sequence set forth in SEQ ID NO: 21. In an embodiment, the isolated antibody comprises a VH sequence set forth in SEQ ID NO: 20 and a VL sequence set forth in SEQ ID NO: 21.

In an embodiment, the isolated antibody is a humanized, human or chimeric antibody. In an embodiment, the isolated antibody is a humanized antibody. In an embodiment, the isolated antibody comprises a heavy chain human constant region of a class selected from IgG, IgA, IgD, IgE, and IgM. In an embodiment, the isolated human Fc region comprises a human heavy chain constant region of the class IgG and a subclass selected from IgG1, IgG2, IgG3, and IgG4. In an embodiment, the human Fc region comprises wild-type, human IgG1 Fc. In an embodiment, the human Fc domain comprises the sequence set forth in SEQ ID NO: 22. In an embodiment, the heavy chain comprises a constant heavy chain sequence set forth by SEQ ID NO: 22. In an embodiment, the light chain comprises a constant light chain sequence set forth by SEQ ID NO: 23. In an embodiment, the antibody comprises the VH sequence set forth in SEQ ID NO: 7, and the VL sequence set forth in SEQ ID NO: 21; and the human Fc region comprises wild-type, human IgG1 Fc. In an embodiment, the antibody comprises the VH sequence set forth in SEQ ID NO: 8, and the VL sequence set forth in SEQ ID NO: 21; and the human Fc region comprises wild-type, human IgG1 Fc. In an embodiment, the isolated antibody comprises the VH sequence set forth in SEQ ID NO: 9, and the VL sequence set forth in SEQ ID NO: 21; and the human Fc region comprises wild-type, human IgG1 Fc. In an embodiment, the isolated antibody comprises the VH sequence set forth in SEQ ID NO: 10, and the VL sequence set forth in SEQ ID NO: 21; and the human Fc region comprises wild-type, human IgG1 Fc. In an embodiment, the isolated antibody comprises the VH sequence set forth in SEQ ID NO: 11, and the VL sequence set forth in SEQ ID NO: 21; and the human Fc region comprises wild-type, human IgG1 Fc. In an embodiment, the isolated antibody comprises the VH sequence set forth in SEQ ID NO: 12, and the VL sequence set forth in SEQ ID NO: 21; and the human Fc region comprises wild-type, human IgG1 Fc. In an embodiment, the isolated antibody comprises the VH sequence set forth in SEQ ID NO: 13, and the VL sequence set forth in SEQ ID NO: 21; and the human Fc region comprises wild-type, human IgG1 Fc. In an embodiment, the isolated antibody comprises the VH sequence set forth in SEQ ID NO: 14, and the VL sequence set forth in SEQ ID NO: 21; and the human Fc region comprises wild-type, human IgG1 Fc. In an embodiment, the isolated antibody comprises the VH sequence set forth in SEQ ID NO: 15, and the VL sequence set forth in SEQ ID NO: 21; and the human Fc region comprises wild-type, human IgG1 Fc. In an embodiment, the isolated antibody comprises the VH sequence set forth in SEQ ID NO: 16, and the VL sequence set forth in SEQ ID NO: 21; and the human Fc region comprises wild-type, human IgG1 Fc. In an embodiment, the isolated antibody comprises the VH sequence set forth in SEQ ID NO: 17, and the VL sequence set forth in SEQ ID NO: 21; and the human Fc region comprises wild-type, human IgG1 Fc. In an embodiment, the isolated antibody comprises the VH sequence set forth in SEQ ID NO: 18, and the VL sequence set forth in SEQ ID NO: 21; and the human Fc region comprises wild-type, human IgG1 Fc. In an embodiment, the isolated antibody comprises the VH sequence set forth in SEQ ID NO: 19, and the VL sequence set forth in SEQ ID NO: 21; and the human Fc region comprises wild-type, human IgG1 Fc. In an embodiment, the isolated antibody comprises the VH sequence set forth in SEQ ID NO: 20, and the VL sequence set forth in SEQ ID NO: 21; and the human Fc region comprises wild-type, human IgG1 Fc.

In an embodiment, the Fc region comprises one or more amino acid substitutions, wherein the one or more substitutions result in increased antibody half-life, increased ADCC activity, increased ADCP activity, or increased CDC activity compared with the Fc without the one or more substitutions.

In an embodiment, the Fc region binds an Fcγ Receptor selected from the group consisting of: FcγRI, FcγRIIa, FcγRIIb, FcγRIIc, FcγRIIIa, and FcγRIIIb. In an embodiment, the isolated antibody is a monoclonal antibody. In an embodiment, the antibody binds an γ377-395 epitope (SEQ ID NO: X) of the fibrin or fibrinogen γC domain.

In an embodiment, the isolated antibody binds to a peptide comprising the sequence of the γ377-395 epitope of the human fibrin or fibrinogen γC domain with a K_{D} of less than or equal to about 1, 2, 3, 4, 5, 6, 7, or 8×10⁻⁵ M, as measured by surface plasmon resonance (SPR) single cycle kinetics (SCK) assay. In an embodiment, the isolated antibody binds to a peptide comprising the sequence of the γ377-395 epitope of the human fibrin or fibrinogen γC domain with a K_{D} of less than or equal to about 8×10-⁵M, as measured by surface plasmon resonance (SPR) single cycle kinetics (SCK) assay.

In an embodiment, the isolated antibody inhibits Mac-1 binding to fibrin or fibrinogen γC domain. In an embodiment, the isolated antibody exhibits inhibition of microglial adhesion to the fibrin or fibrinogen γC domain.

In an embodiment, the isolated antibody is used in the treatment of a degenerative disorder of the nervous system.

In certain aspects, described herein is an isolated polynucleotide or set of polynucleotides encoding the antibody of any of the above claims, a VH thereof, a VL thereof, a light chain thereof, a heavy chain thereof, or an antigen-binding portion thereof; optionally cDNA.

In certain aspects, described herein is a vector or set of vectors comprising the polynucleotide or set of polynucleotides described herein.

In certain aspects, described herein is a host cell comprising the polynucleotide or set of polynucleotides encoding the isolated antibody or the vector or set of vectors comprising the polynucleotides encoding the isolated antibody.

In certain aspects, described herein is a method of producing an antibody, the method comprising expressing the antibody with the host cell comprising the polynucleotide or set of polynucleotide encoding the isolated antibody or the vector or set of vectors comprising the polynucleotides encoding the isolated antibody and isolating the expressed antibody.

In certain aspects, described herein is a pharmaceutical composition comprising the isolated antibody and a pharmaceutically acceptable excipient.

In certain aspects, described herein is a kit comprising the isolated antibody or a pharmaceutical composition comprising the isolated antibody and instructions for use

In certain aspects, described herein is a method for treating a degenerative disorder of the nervous system, the method comprising administering to a mammalian subject a therapeutically effective amount the isolated antibody or pharmaceutical composition comprising the isolated antibody. In certain embodiments, the degenerative disorder of the nervous system is selected from the group consisting of: multiple sclerosis, spinal cord injury, stroke, and Alzheimer's Disease.

In certain aspects, described herein is a method for treating a pathology associated with Mac-1 binding to fibrin or Mac-1 binding with fibrinogen, the method comprising administering to a mammalian subject a therapeutically effective amount an isolated antibody or a pharmaceutical composition comprising an isolated antibody described herein.

In certain aspects, described herein is a method of inhibiting microglia activation, the method comprising administering to a mammalian subject a therapeutically effective amount an isolated antibody or a pharmaceutical composition comprising an isolated antibody described herein.

In certain aspects, described herein is a method of preventing a degenerative disorder of the nervous system, the method comprising administering to a mammalian subject a therapeutically effective amount of an isolated antibody or a pharmaceutical composition comprising an isolated antibody described herein.

In certain aspects, described herein is a method of treating colitis, the method comprising administering to a mammalian subject a therapeutically effective amount of an isolated antibody or a pharmaceutical composition comprising an isolated antibody described herein. In certain aspects, described herein is a method of preventing colitis, the method comprising administering to a mammalian subject a therapeutically effective amount of an isolated antibody or a pharmaceutical composition comprising an isolated antibody described herein.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 is a diagram showing humanized variant IgG1 antibodies designed with 6 variant heavy chains and 5 variant light chains. VH0 and Vk0 corresponds to the variable heavy and variable light chain original chimeric antibody harboring the original anti-fibrin 5B8 monoclonal antibody CDRs.

FIG. 2 is a diagram showing a sequence alignment of chimeric antibody (VH0 and Vk0) and humanized variant sequences.

FIG. 3 is a diagram illustrating the amino acid residue differences between VH3 and VH4. The positions that are different between VH3 and VH4 are highlighted.

FIG. 5 are images of results of non-reducing SDS-PAGE analysis performed on purified antibody proteins, verifying concentrations of antibodies for affinity analysis.

FIG. 6: is a graph showing the results of the thermostability analysis indicating the first (Tₘ1) and second thermal transitions (Tₘ2) for six humanized antibody variants.

FIG. 7A: is a graph showing the results of ELISAs performed using mouse monoclonal 5B8 antibody binding to human Fibrinogen and human Fibrin.

FIG. 7B: is a graph showing the results of ELISAs performed using mouse monoclonal 5B8 antibody binding to human P2 peptide.

FIG. 8A: is a graph showing the results of ELISAs performed using mouse IgG2b isotype control antibody binding to human Fibrinogen and human Fibrin.

FIG. 8B: is a graph showing the results of ELISAs performed using mouse IgG2b isotype control antibody binding to human P2 peptide.

FIG. 9A: is a graph showing the results of ELISAs performed using mouse IgG1 isotype control antibody binding to human Fibrinogen and human Fibrin.

FIG. 9B: is a graph showing the results of ELISAs performed using mouse IgG1 isotype control antibody binding to human P2 peptide.

FIG. 10A: is a graph showing the results of ELISAs performed using mouse chimeric VH0Vk0 antibody (chimeric mouse monoclonal 5B8 antibody with human Fc) binding to human Fibrinogen and human Fibrin.

FIG. 10B: is a graph showing the results of ELISAs performed using mouse chimeric VH0Vk0 antibody (chimeric mouse monoclonal 5B8 antibody with human Fc) binding to human P2 peptide.

FIG. 11A: is a graph showing the results of ELISAs performed using humanized VH3Vk1 antibody binding to human Fibrinogen and human Fibrin.

FIG. 11B: is a graph showing the results of ELISAs performed using humanized VH3Vk1 antibody binding to human P2 peptide.

FIG. 12A: is a graph showing the results of ELISAs performed using humanized VH3Vk2 antibody binding to human Fibrinogen and human Fibrin.

FIG. 12B: is a graph showing the results of ELISAs performed using humanized VH3Vk2 antibody binding to human P2 peptide.

FIG. 13A: is a graph showing the results of ELISAs performed using humanized VH4Vk1 antibody binding to human Fibrinogen and human Fibrin.

FIG. 13B: is a graph showing the results of ELISAs performed using humanized VH4Vk1 antibody binding to human P2 peptide.

FIG. 14A: is a graph showing the results of ELISAs performed using humanized VH4Vk2 antibody binding to human Fibrinogen and human Fibrin.

FIG. 14B: is a graph showing the results of ELISAs performed using humanized VH4Vk2 antibody binding to human P2 peptide.

FIG. 15A: is a graph showing the results of ELISAs performed using humanized VH5Vk2 antibody binding to human Fibrinogen and human Fibrin.

FIG. 15B: is a graph showing the results of ELISAs performed using humanized VH5Vk2 antibody binding to human P2 peptide.

FIG. 16A: is a graph showing the results of ELISAs performed using humanized VH6Vk2 antibody binding to human Fibrinogen and human Fibrin.

FIG. 16B: is a graph showing the results of ELISAs performed using humanized VH6Vk2 antibody binding to human P2 peptide.

FIG. 17A: is a graph showing results of coagulation assays controls.

FIG. 17B: is a graph showing results of coagulation assays with 20 ug of select humanized antibodies.

FIG. 18 is a diagram illustrating the procedure for affinity determination by steady state analysis using single cycle kinetics.

FIG. 19 are graphs showing results of SEC-HPLC analysis of select humanized variants.

FIG. 20 are graphs showing the raw and fitted data from multicycle kinetics analyses of select variants.

FIG. 21 is a diagram illustrating potential amino acid residue liabilities within the VH CDR sequences in the humanized variant antibodies.

FIG. 22 is a graph illustrating the capture levels obtained by manual loading of the HEK cell supernatants for Biacore steady state analysis for the chimeric antibody, the original humanized variant antibodies (VH4/Vk2) and (VH5Vk2) with cysteine 102, and the twelve humanized variant antibodies with the cysteine substitutions.

FIG. 23 shows the results of the steady state analysis using single cycle kinetics for twelve humanized variant antibodies with the cysteine substitutions.

FIG. 24 is a graph showing the effect of I.C.V. injection of humanized anti-fibrin antibody variant VH5 C102G/VK2 on microglia activation, oxidative stress, and macrophage recruitment was assessed. 10 ug antibody was administered prophylactically by i.e.v. injection to FIE mice (FIG. 24). Each circle represents an individual animal. Data are mean±s.e.m. One-way ANOVA with Tukey's multiple comparisons.

FIG. 25 are images of tissue sections from mice with chronic EAE harboring fibrinogen accumulation in spinal cord lesions that were stained with 10 mg/ml VH5 C102G/VK2-biotin and CY3-streptavidin antibody.

FIG. 26 is a graph showing a pharmacokinetic profile of humanized anti-fibrin antibody variant VH5 C102G/VK2. Antibodies were detected via ELISA in plasma from EAE mice that have been administered either 10 mg/Kg or 30 mg/Kg VH5 C102G/VK2 antibody.

FIG. 27 is a graph showing a pharmacokinetic profile of humanized anti-fibrin antibody variant VH5 C102G/VK2. Antibodies were detected via ELISA in plasma and blood from wild-type Balb/c mice that have been administered VH5 C102G/VK2 antibody.

FIG. 28A is a graph showing reduced microglia in mice with fibrinogen-induced encephalomyelitis (FIE) that have been administered either 10 mg/kg or 30 mg/kg VH5 C102G/VK2 humanized antibody. Tissues were stained with Iba-1 (microglia marker, at a dilution of 1:750). The immunoreactivity of Iba-1 (Iba-1+ area) was then calculated.

FIG. 28B is a graph showing reduced macrophage infiltration in mice with fibrinogen-induced encephalomyelitis (FIE) that have been administered either 10 mg/kg or 30 mg/kg VH5 C102G/VK2 humanized antibody. Tissues were stained with Mac-2 (macrophage infiltration marker, at a dilution of 1:750). The immunoreactivity of Mac-2 (Mac-2+ area) was then calculated.

FIG. 29 is a graph showing clinical score of PLP EAE in mice that were prophylactically injected with humanized anti-fibrin antibodies (5 mg/kg i.p. every 3 days) N=10.

FIG. 30A is a graph showing time to onset of disease of PLP EAE in mice that were prophylactically injected with humanized anti-fibrin antibodies (5 mg/kg i.p. every 3 days) N=10.

FIG. 30B is a graph showing paralysis rate of PLP EAE in mice that were prophylactically injected with humanized anti-fibrin antibodies (5 mg/kg i.p. every 3 days) N=10.

### DETAILED DESCRIPTION

### Definitions

Unless otherwise defined, all terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular form “a”, “an”, and “the” includes plural references unless indicated otherwise.

It is understood that aspects and embodiments of the invention described herein include “comprising,” “consisting,” and “consisting essentially of” aspects and embodiments.

For all compositions described herein, and all methods using a composition described herein, the compositions can either comprise the listed components or steps, or can “consist essentially of” the listed components or steps. When a composition is described as “consisting essentially of” the listed components, the composition contains the components listed, and may contain other components which do not substantially affect the condition being treated, but do not contain any other components which substantially affect the condition being treated other than those components expressly listed; or, if the composition does contain extra components other than those listed which substantially affect the condition being treated, the composition does not contain a sufficient concentration or amount of the extra components to substantially affect the condition being treated. When a method is described as “consisting essentially of” the listed steps, the method contains the steps listed, and may contain other steps that do not substantially affect the condition being treated, but the method does not contain any other steps which substantially affect the condition being treated other than those steps expressly listed. As a non-limiting specific example, when a composition is described as ‘consisting essentially of’ a component, the composition may additionally contain any amount of pharmaceutically acceptable carriers, vehicles, or diluents and other such components which do not substantially affect the condition being treated.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which an exogenous nucleic acid has been introduced, and the progeny of such cells. Host cells include “transformants” (or “transformed cells”) and “transfectants” (or “transfected cells”), which each include the primary transformed or transfected cell and progeny derived therefrom. Such progeny may not be completely identical in nucleic acid content to a parent cell, and may contain mutations. A “recombinant host cell” or “host cell” refers to a cell that includes an exogenous polynucleotide, regardless of the method used for insertion, for example, direct uptake, transduction, f-mating, or other methods known in the art to create recombinant host cells.

As used herein, the term “eukaryote” refers to organisms belonging to the phylogenetic domain Eucarya such as animals (including but not limited to, mammals, insects, reptiles, birds, etc.), ciliates, plants (including but not limited to, monocots, dicots, algae, etc.), fungi, yeasts, flagellates, microsporidia, protists, etc.

As used herein, the term “prokaryote” refers to prokaryotic organisms. For example, a non-eukaryotic organism can belong to the Eubacteria (including but not limited to, Escherichia coli, Thermus thermophilus, Bacillus stearothermophilus, Pseudomonas fluorescens, Pseudomonas aeruginosa, Pseudomonas putida, etc.) phylogenetic domain, or the Archaea (including but not limited to, Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, etc.) phylogenetic domain.

An “effective amount” or “therapeutically effective amount” as used herein refers to an amount of therapeutic compound, such as an anti-FIBRIN antibody, administered to an individual, either as a single dose or as part of a series of doses, which is effective to produce or contribute to a desired therapeutic effect, either alone or in combination with another therapeutic modality. Examples of a desired therapeutic effect is enhancing an immune response, slowing or delaying tumor development; stabilization of disease; amelioration of one or more symptoms. An effective amount may be given in one or more dosages.

The term “treating” (and variations thereof such as “treat” or “treatment”) refers to clinical intervention in an attempt to alter the natural course of a disease or condition in a subject in need thereof. Treatment can be performed during the course of clinical pathology. Desirable effects of treatment include preventing recurrence of disease, alleviation of symptoms, diminishment of any direct or indirect pathological consequences of the disease, preventing metastasis, decreasing the rate of disease progression, amelioration or palliation of the disease state, and remission or improved prognosis.

The term “sufficient amount” means an amount sufficient to produce a desired effect, e.g., an amount sufficient to modulate an immune response in a subject.

As used herein, the term “subject” or “individual” means a mammalian subject. Exemplary subjects include humans, monkeys, dogs, cats, mice, rats, cows, horses, camels, goats, rabbits, and sheep. In certain embodiments, the subject is a human. In some embodiments the subject has a disease or condition that can be treated with an antibody provided herein. In some aspects, the disease or condition is a cancer. In some aspects, the disease or condition is a viral infection.

The term “in vitro” refers to processes that occur in a living cell growing separate from a living organism, e.g., growing in tissue culture.

The term “in vivo” refers to processes that occur in a living organism.

The term “package insert” is used to refer to instructions customarily included in commercial packages of therapeutic or diagnostic products (e.g., kits) that contain information about the indications, usage, dosage, administration, combination therapy, contraindications and/or warnings concerning the use of such therapeutic or diagnostic products.

The term “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

The terms “co-administration”, “co-administer”, and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to the administration of a second therapeutic agent.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

The term “about” indicates and encompasses an indicated value and a range above and below that value. In certain embodiments, the term “about” indicates the designated value±10%, ±5%, or ±1%. In certain embodiments, where applicable, the term “about” indicates the designated value(s)±one standard deviation of that value(s).

The term “agonize” refers to the activation of receptor signaling to induce a biological response associated with activation of the receptor. An “agonist” is an entity that binds to and agonizes a receptor.

The term “antagonize” refers to the inhibition of receptor signaling to inhibit a biological response associated with activation of the receptor. An “antagonist” is an entity that binds to and antagonizes a receptor.

For any of the structural and functional characteristics described herein, methods of determining these characteristics are known in the art.

The term “optionally” is meant, when used sequentially, to include from one to all of the enumerated combinations and contemplates all sub-combinations.

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

The term “affinity” refers to the strength of the sum total of non-covalent interactions between a single binding site of a molecule (e.g., an antibody) and its binding partner (e.g., an antigen or epitope). Unless indicated otherwise, as used herein, “affinity” refers to intrinsic binding affinity, which reflects a 1:1 interaction between members of a binding pair (e.g., antibody and antigen or epitope).

The term “k_{d}” (sec⁻¹), as used herein, refers to the dissociation rate constant of a particular antibody-antigen interaction. This value is also referred to as the k_{off} value.

The term “k_{d}” (M⁻¹×sec⁻¹), as used herein, refers to the association rate constant of a particular antibody-antigen interaction. This value is also referred to as the kₒₙ value.

The term “K_{D}” (M), as used herein, refers to the dissociation equilibrium constant of a particular antibody-antigen interaction. K_{D}=k_{d}/kₐ. In some embodiments, the affinity of an antibody is described in terms of the K_{D} for an interaction between such antibody and its antigen. For clarity, as known in the art, a smaller K_{D} value indicates a higher affinity interaction, while a larger K_{D} value indicates a lower affinity interaction.

The term “K_{A}” (M⁻¹), as used herein, refers to the association equilibrium constant of a particular antibody-antigen interaction. K_{A}=kₐ/k_{d}.

The term “antibody” is used herein in its broadest sense and includes certain types of immunoglobulin molecules comprising one or more antigen-binding domains that specifically bind to an antigen or epitope. An antibody specifically includes intact antibodies (e.g., intact immunoglobulins), antibody fragments, and multi-specific antibodies.

A “Fibrin antibody,” “anti-Fibrin antibody,” or “Fibrin-specific antibody” is an antibody, as provided herein, which specifically binds to the antigen Fibrin. In some embodiments, the antibody binds the extracellular domain of Fibrin. In certain embodiments, a Fibrin antibody provided herein binds to an epitope of Fibrin that is conserved between or among Fibrin proteins from different species.

The term “epitope” means a portion of an antigen that specifically binds to an antibody.

The term “hypervariable region” or “HVR”, as used herein, refers to each of the regions of an antibody variable domain which are hypervariable in sequence and/or form structurally defined loops (“hypervariable loops”).

The term “antigen-binding domain” means the portion of an antibody that is capable of specifically binding to an antigen or epitope.

The term “chimeric antibody” refers to an antibody in which a portion of the heavy and/or light chain is derived from a particular source or species, while the remainder of the heavy and/or light chain is derived from a different source or species.

The term “human antibody” refers to an antibody which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

The term “humanized antibody” refers to a protein having a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject.

The term “multispecific antibody” refers to an antibody that comprises two or more different antigen-binding domains that collectively specifically bind two or more different epitopes.

A “monospecific antibody” is an antibody that comprises one or more binding sites that specifically bind to a single epitope. An example of a monospecific antibody is a naturally occurring IgG molecule which, while divalent (i.e., having two antigen-binding domains), recognizes the same epitope at each of the two antigen-binding domains. The binding specificity may be present in any suitable valency.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “single-chain” refers to a molecule comprising amino acid monomers linearly linked by peptide bonds. In a particular such embodiment, the C-terminus of the Fab light chain is connected to the N-terminus of the Fab heavy chain in the single-chain Fab molecule. As described in more detail herein, an scFv has a variable domain of light chain (VL) connected from its C-terminus to the N-terminal end of a variable domain of heavy chain (VH) by a polypeptide chain. Alternately the scFv comprises of polypeptide chain where in the C-terminal end of the VH is connected to the N-terminal end of VL by a polypeptide chain.

The “Fab fragment” (also referred to as fragment antigen-binding) contains the constant domain (CL) of the light chain and the first constant domain (CH1) of the heavy chain along with the variable domains VL and VH on the light and heavy chains respectively. The variable domains comprise the complementarity determining loops (CDR, also referred to as hypervariable region) that are involved in antigen-binding. Fab′ fragments differ from Fab fragments by the addition of a few residues at the carboxy terminus of the heavy chain CH1 domain including one or more cysteines from the antibody hinge region.

“F(ab′)2” fragments contain two Fab′ fragments joined, near the hinge region, by disulfide bonds. F(ab′)2 fragments may be generated, for example, by recombinant methods or by pepsin digestion of an intact antibody. The F(ab′) fragments can be dissociated, for example, by treatment with β-mercaptoethanol.

“Fv” fragments comprise a non-covalently-linked dimer of one heavy chain variable domain and one light chain variable domain.

“Single-chain Fv” or “sFv” or “scFv” includes the VH and VL domains of an antibody, wherein these domains are present in a single polypeptide chain. In one embodiment, the Fv polypeptide further comprises a polypeptide linker between the VH and VL domains which enables the scFv to form the desired structure for antigen-binding. For a review of scFv see Pluckthun in The Pharmacology of Monoclonal Antibodies, vol. 113, Rosenburg and Moore eds., Springer-Verlag, New York, pp. 269-315 (1994). HER2 antibody scFv fragments are described in WO93/16185; U.S. Pat. Nos. 5,571,894; and 5,587,458.

“scFv-Fc” fragments comprise an scFv attached to an Fc domain. For example, an Fc domain may be attached to the C-terminal of the scFv. The Fc domain may follow the VH or VL, depending on the orientation of the variable domains in the scFv (i.e., VH-VL or VL-VH). Any suitable Fc domain known in the art or described herein may be used. In some cases, the Fc domain comprises an IgG4 Fc domain.

The term “single domain antibody” or “sdAb” refers to a molecule in which one variable domain of an antibody specifically binds to an antigen without the presence of the other variable domain. Single domain antibodies, and fragments thereof, are described in Arabi Ghahroudi et al., FEBS Letters, 1998, 414:521-526 and Muyldermans et al., Trends in Biochem. Sci., 2001, 26:230-245, each of which is incorporated by reference in its entirety. Single domain antibodies are also known as sdAbs or nanobodies. Sdabs are fairly stable and easy to express as fusion partner with the Fc chain of an antibody (Harmsen M M, De Haard HJ (2007). “Properties, production, and applications of camelid single-domain antibody fragments”. Appl. Microbiol Biotechnol. 77(1): 13-22).

The terms “full length antibody,” “intact antibody,” and “whole antibody” are used herein interchangeably to refer to an antibody having a structure substantially similar to a naturally occurring antibody structure and having heavy chains that comprise an Fc region. For example, when used to refer to an IgG molecule, a “full length antibody” is an antibody that comprises two heavy chains and two light chains.

The term “antibody fragment” refers to an antibody that comprises a portion of an intact antibody, such as the antigen-binding or variable region of an intact antibody. Antibody fragments include, for example, Fv fragments, Fab fragments, F(ab′)2 fragments, Fab′ fragments, scFv (sFv) fragments, and scFv-Fc fragments.

The term “Fc domain” or “Fc region” herein is used to define a C-terminal region of an immunoglobulin heavy chain that contains at least a portion of the constant region. The term includes native sequence Fc regions and variant Fc regions.

The term “substantially purified” refers to a construct described herein, or variant thereof that may be substantially or essentially free of components that normally accompany or interact with the protein as found in its naturally occurring environment, i.e. a native cell, or host cell in the case of recombinantly produced heteromultimer that in certain embodiments, is substantially free of cellular material includes preparations of protein having less than about 30%, less than about 25%, less than about 20%, less than about 15%, less than about 10%, less than about 5%, less than about 4%, less than about 3%, less than about 2%, or less than about 1% (by dry weight) of contaminating protein.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., using publicly available computer software such as BLAST, BLASTP, BLASTN, BLAST-2, ALIGN, MEGALIGN (DNASTAR), CLUSTALW, CLUSTAL OMEGA, or MUSCLE software or other algorithms available to persons of skill) or by visual inspection. Software for performing BLAST analyses is publicly available through the National Center for Biotechnology Information (ncbi.nlm.nih.gov). Those skilled in the art can determine appropriate parameters for aligning sequences, including any algorithms needed to achieve maximal alignment over the full length of the sequences being compared. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

It must be noted that, as used in the specification and the appended claims, the singular forms “a,” “an” and “the” include plural referents unless the context clearly dictates otherwise.

### Anti-Fibrin Antibodies

### Antibody Structure

The present application provides antibodies and compositions comprising an antibody which binds a fibrin protein.

The recognized immunoglobulin genes include the kappa, lambda, alpha, gamma, delta, epsilon and mu constant region genes, as well as the myriad immunoglobulin variable region genes. Light chains are classified as either kappa or lambda. The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG₂, IgG₃, IgG₄, IgA1, and IgA₂. The heavy chain constant domains that correspond to the different classes of immunoglobulins are called α, δ, ε, γ, and μ, respectively.

An exemplary immunoglobulin (antibody) structural unit is composed of two pairs of polypeptide chains, each pair having one “light” (about 25 kD) and one “heavy” chain (about 50-70 kD). The N-terminal domain of each chain defines a variable region of about 100 to 110 or more amino acids primarily responsible for antigen recognition. The terms variable light chain (VL) and variable heavy chain (VH) refer to these light and heavy chain domains respectively. The IgG1 heavy chain comprises of the VH, CH1, CH2 and CH3 domains respectively from the N to C-terminus. The light chain comprises of the VL and CL domains from N to C terminus. The IgG1 heavy chain comprises a hinge between the CH1 and CH2 domains. In certain embodiments, the immunoglobulin constructs comprise at least one immunoglobulin domain from IgG, IgM, IgA, IgD, or IgE connected to a therapeutic polypeptide. In some embodiments, the immunoglobulin domain found in an antibody provided herein, is from or derived from an immunoglobulin based construct such as a diabody, or a nanobody. In certain embodiments, the immunoglobulin constructs described herein comprise at least one immunoglobulin domain from a heavy chain antibody such as a camelid antibody. In certain embodiments, the immunoglobulin constructs provided herein comprise at least one immunoglobulin domain from a mammalian antibody such as a bovine antibody, a human antibody, a camelid antibody, a mouse antibody or any chimeric antibody.

In some embodiments, the antibodies provided herein comprise a heavy chain. In one embodiment, the heavy chain is an IgA. In one embodiment, the heavy chain is an IgD. In one embodiment, the heavy chain is an IgE. In one embodiment, the heavy chain is an IgG. In one embodiment, the heavy chain is an IgM. In one embodiment, the heavy chain is an IgG1. In one embodiment, the heavy chain is an IgG2. In one embodiment, the heavy chain is an IgG3. In one embodiment, the heavy chain is an IgG4. In one embodiment, the heavy chain is an IgA1. In one embodiment, the heavy chain is an IgA2.

In some embodiments, an antibody is an IgG1 antibody. In some embodiments, an antibody is an IgG3 antibody. In some embodiments, an antibody is an IgG2 antibody. In some embodiments, an antibody is an IgG4 antibody.

Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen-binding regions. This particular region has been described by Kabat et al., U.S. Dept. of Health and Human Services, Sequences of Proteins of Immunological Interest (1983) and by Chothia et al., J Mol Biol 196:901-917 (1987), where the definitions include overlapping or subsets of amino acid residues when compared against each other. Nevertheless, application of either definition to refer to a CDR of an antibody or variants thereof is intended to be within the scope of the term as defined and used herein. The exact residue numbers which encompass a particular CDR will vary depending on the sequence and size of the CDR. Those skilled in the art can routinely determine which residues comprise a particular CDR given the variable region amino acid sequence of the antibody.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“AHo” numbering scheme); each of which is incorporated by reference in its entirety.

Table A provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

CDRs may be assigned, for example, using antibody numbering software, such as Abnum, available at www.bioinf.org.uk/abs/abnum/, and described in Abhinandan and Martin, Immunology, 2008, 45:3832-3839, incorporated by reference in its entirety.

TABLE A
Residues in CDRs according to Kabat and Chothia numbering schemes.
Table A

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat Numbering) | H31-H35B | H26-H32 or H34* |
| H1 (Chothia Numbering) | H31-H35 | H26-H32 |
| H2 | H50-H65 | H52-H56 |
| H3 | H95-H102 | H95-H102 |

*The C-terminus of CDR-H1, when numbered using the Kabat numbering convention, varies between H32 and H34, depending on the length of the CDR.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

One example of an antigen-binding domain is an antigen-binding domain formed by a VH-VL dimer of an antibody. Another example of an antigen-binding domain is an antigen-binding domain formed by diversification of certain loops from the tenth fibronectin type III domain of an Adnectin. An antigen-binding domain can include CDRs 1, 2, and 3 from a heavy chain in that order; and CDRs 1, 2, and 3 from a light chain in that order.

Epitopes frequently consist of surface-accessible amino acid residues and/or sugar side chains and may have specific three-dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. The epitope to which an antibody binds can be determined using known techniques for epitope determination such as, for example, testing for antibody binding to Fibrin variants with different point-mutations, or to chimeric Fibrin variants.

To screen for antibodies which bind to an epitope on a target antigen bound by an antibody of interest (e.g., Fibrin), a routine cross-blocking assay such as that described in Antibodies, A Laboratory Manual, Cold Spring Harbor Laboratory, Ed Harlow and David Lane (1988), can be performed. Alternatively, or additionally, epitope mapping can be performed by methods known in the art.

Chimeric antibodies are antibodies in which a portion of the heavy and/or light chain is derived from a particular source or species, while the remainder of the heavy and/or light chain is derived from a different source or species.

Human antibodies are antibodies which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

A humanized antibody has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies can be found in U.S. Pat. Nos. 6,054,297, 5,886,152 and 5,877,293. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

The two or more different epitopes may be epitopes on the same antigen (e.g., a single Fibrin molecule expressed by a cell) or on different antigens (e.g., different Fibrin molecules expressed by the same cell, or a Fibrin molecule and a non-Fibrin molecule). In some aspects, a multi-specific antibody binds two different epitopes (i.e., a “bispecific antibody”). In some aspects, a multi-specific antibody binds three different epitopes (i.e., a “trispecific antibody”).

Anti-Fibrin antibodies can include those described herein such as the clones set forth in the drawings and/or tables. In some embodiments, the antibody comprises an alternative scaffold. In some embodiments, the antibody consists of an alternative scaffold. In some embodiments, the antibody consists essentially of an alternative scaffold. In some embodiments, the antibody comprises an antibody fragment. In some embodiments, the antibody consists of an antibody fragment. In some embodiments, the antibody consists essentially of an antibody fragment.

In some embodiments the antibodies are monoclonal antibodies.

In some embodiments the antibodies are polyclonal antibodies.

In some embodiments the antibodies are produced by hybridomas. In other embodiments, the antibodies are produced by recombinant cells engineered to express the desired variable and constant domains.

In some embodiments the antibodies may be single chain antibodies or other antibody derivatives retaining the antigen specificity and the lower hinge region or a variant thereof.

In some embodiments the antibodies may be polyfunctional antibodies, recombinant antibodies, human antibodies, humanized antibodies, fragments or variants thereof. In particular embodiments, the antibody fragment or a derivative thereof is selected from a Fab fragment, a Fab′2 fragment, a CDR and ScFv.

In some embodiments, the antibodies are capable of forming an immune complex. For example, an immune complex can be a tumor cell covered by antibodies.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

### Sequences of Fibrin Antibodies

### V_{H} Domains

In some embodiments, an antibody provided herein comprises a V_{H} sequence selected from SEQ ID NOs: 7, 8, 9, 10, 11, 12, 13, 14, 15, 16, 17, 18, 19, and 20. In some embodiments, an antibody provided herein comprises a V_{H} sequence of SEQ ID NO: 7. In some embodiments, an antibody provided herein comprises a V_{H} sequence of SEQ ID NO: 8. In some embodiments, an antibody provided herein comprises a V_{H} sequence of SEQ ID NO: 9. In some embodiments, an antibody provided herein comprises a V_{H} sequence of SEQ ID NO: 10. In some embodiments, an antibody provided herein comprises a V_{H} sequence of SEQ ID NO: 11. In some embodiments, an antibody provided herein comprises a V_{H} sequence of SEQ ID NO: 12. In some embodiments, an antibody provided herein comprises a V_{H} sequence of SEQ ID NO: 13. In some embodiments, an antibody provided herein comprises a V_{H} sequence of SEQ ID NO: 14. In some embodiments, an antibody provided herein comprises a V_{H} sequence of SEQ ID NO: 15. In some embodiments, an antibody provided herein comprises a V_{H} sequence of SEQ ID NO: 16. In some embodiments, an antibody provided herein comprises a V_{H} sequence of SEQ ID NO: 17. In some embodiments, an antibody provided herein comprises a V_{H} sequence of SEQ ID NO: 18. In some embodiments, an antibody provided herein comprises a V_{H} sequence of SEQ ID NO: 19. In some embodiments, an antibody provided herein comprises a V_{H} sequence of SEQ ID NO: 20.

In some embodiments, an antibody provided herein comprises a V_{H} sequence having at least about 50%, 60%, 70%, 80%, 90%, 95%, or 99% identity to an illustrative V_{H} sequence provided in SEQ ID NOs: 7, 8, 9, 10, 11, 12, 13, 14, 15, 16, 17, 18, 19, and 20. In some embodiments, an antibody provided herein comprises a V_{H} sequence provided in SEQ ID NOs: 7, 8, 9, 10, 11, 12, 13, 14, 15, 16, 17, 18, 19, and 20, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

### V_{L} Domains

In some embodiments, an antibody provided herein comprises a V_{L} sequence selected from SEQ ID NO: 21.

In some embodiments, an antibody provided herein comprises a V_{L} sequence having at least about 50%, 60%, 70%, 80%, 90%, 95%, or 99% identity to an illustrative V_{L} sequence provided in SEQ ID NO: 21. In some embodiments, an antibody provided herein comprises a V_{L} sequence provided in SEQ ID NO: 21 with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

### VH-VL Combinations

In some embodiments, an antibody provided herein comprises a V_{H} sequence selected from SEQ ID NOs: 7, 8, 9, 10, 11, 12, 13, 14, 15, 16, 17, 18, 19, and 20; and a V_{L} sequence selected from SEQ ID NO: 21.

In some embodiments, an antibody provided herein comprises a V_{H} sequence of SEQ ID NO: 7 and a V_{L} sequence of SEQ ID NO: 21. In some embodiments, an antibody provided herein comprises a V_{H} sequence of SEQ ID NO: 8 and a V_{L} sequence of SEQ ID NO:21. In some embodiments, an antibody provided herein comprises a V_{H} sequence of SEQ ID NO: 9 and a V_{L} sequence of SEQ ID NO:21. In some embodiments, an antibody provided herein comprises a V_{H} sequence of SEQ ID NO: 10 and a V_{L} sequence of SEQ ID NO: 21. In some embodiments, an antibody provided herein comprises a V_{H} sequence of SEQ ID NO: 11 and a V_{L} sequence of SEQ ID NO: 21. In some embodiments, an antibody provided herein comprises a V_{H} sequence of SEQ ID NO: 12 and a V_{L} sequence of SEQ ID NO: 21. In some embodiments, an antibody provided herein comprises a V_{H} sequence of SEQ ID NO: 13 and a V_{L} sequence of SEQ ID NO: 21. In some embodiments, an antibody provided herein comprises a V_{H} sequence of SEQ ID NO: 14 and a V_{L} sequence of SEQ ID NO: 21. In some embodiments, an antibody provided herein comprises a V_{H} sequence of SEQ ID NO: 15 and a V_{L} sequence of SEQ ID NO: 21. In some embodiments, an antibody provided herein comprises a V_{H} sequence of SEQ ID NO: 16 and a V_{L} sequence of SEQ ID NO: 21. In some embodiments, an antibody provided herein comprises a V_{H} sequence of SEQ ID NO: 17 and a V_{L} sequence of SEQ ID NO: 21. In some embodiments, an antibody provided herein comprises a V_{H} sequence of SEQ ID NO: 18 and a V_{L} sequence of SEQ ID NO: 21. In some embodiments, an antibody provided herein comprises a V_{H} sequence of SEQ ID NO: 19 and a V_{L} sequence of SEQ ID NO:21. In some embodiments, an antibody provided herein comprises a V_{H} sequence of SEQ ID NO:20 and a V_{L} sequence of SEQ ID NO:21.

In certain aspects, any of SEQ ID NOs: 7, 8, 9, 10, 11, 12, 13, 14, 15, 16, 17, 18, 19, and 20 can be combined with any of SEQ ID NO: 21.

In some embodiments, an antibody provided herein comprises a V_{H} sequence having at least about 50%, 60%, 70%, 80%, 90%, 95%, or 99% identity to an illustrative V_{H} sequence provided in SEQ ID NOs: 7, 8, 9, 10, 11, 12, 13, 14, 15, 16, 17, 18, 19, and 20; and a V_{L} sequence having at least about 50%, 60%, 70%, 80%, 90%, 95%, or 99% identity to an illustrative V_{L} sequence provided in SEQ ID NO: 21. In some embodiments, an antibody provided herein comprises a V_{H} sequence provided in SEQ ID NOs: 7, 8, 9, 10, 11, 12, 13, 14, 15, 16, 17, 18, 19, and 20, with up to 1, 2, 3, 4, 5, 6, 7, 8, 9, 10, 11, 12, 13, 14, 15, 16, 17, 18, 19, 20, 21, 22, 23, 24, or 25 amino acid substitutions, and a V_{L} sequence provided in SEQ ID NO: 21, with up to 1, 2, 3, 4, 5, 6, 7, 8, 9, 10, 11, 12, 13, 14, 15, 16, 17, 18, 19, 20, 21, 22, 23, 24, or 25 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

### CDRs

In some embodiments, an antibody provided herein comprises one to three CDRs of a V_{H} domain selected from SEQ ID NOs: 7, 8, 9, 10, 11, 12, 13, 14, 15, 16, 17, 18, 19, and 20. In some embodiments, an antibody provided herein comprises two to three CDRs of a V_{H} domain selected from SEQ ID NOs: 7, 8, 9, 10, 11, 12, 13, 14, 15, 16, 17, 18, 19, and 20. In some embodiments, an antibody provided herein comprises three CDRs of a V_{H} domain selected from SEQ ID NOs: 37, 8, 9, 10, 11, 12, 13, 14, 15, 16, 17, 18, 19, and 20. In some aspects, the CDRs are Exemplary CDRs. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, the CDRs are CDRs having at least about 50%, 75%, 80%, 85%, 90%, or 95% identity with a CDR-H1, CDR-H2, or CDR-H3 of SEQ ID NOs: 7, 8, 9, 10, 11, 12, 13, 14, 15, 16, 17, 18, 19, and 20. In some embodiments, the CDR-H1 is a CDR-H1 of a V_{H} domain selected from SEQ ID NOs: 7, 8, 9, 10, 11, 12, 13, 14, 15, 16, 17, 18, 19, and 20, with up to 1, 2, 3, 4, or 5 amino acid substitutions. In some embodiments, the CDR-H2 is a CDR-H2 of a V_{H} domain selected from SEQ ID NOs: 7, 8, 9, 10, 11, 12, 13, 14, 15, 16, 17, 18, 19, and 20, with up to 1, 2, 3, 4, 5, 6, 7, or 8 amino acid substitutions. In some embodiments, the CDR-H3 is a CDR-H3 of a V_{H} domain selected from SEQ ID NOs: 7, 8, 9, 10, 11, 12, 13, 14, 15, 16, 17, 18, 19, and 20, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises one to three CDRs of a VL domain of SEQ ID NO: 21. In some embodiments, an antibody provided herein comprises two to three CDRs of a VL domain of SEQ ID NO: 21. In some embodiments, an antibody provided herein comprises three CDRs of a VL domain of SEQ ID NO: 21. In some aspects, the CDRs are Exemplary CDRs. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, the CDRs are CDRs having at least about 50%, 75%, 80%, 85%, 90%, or 95% identity with a CDR-L1, CDR-L2, or CDR-L3 of SEQ ID NO: 21. In some embodiments, the CDR-L1 is a CDR-L1 of a VL domain of SEQ ID NO: 21, with up to 1, 2, 3, 4, or 5 amino acid substitutions. In some embodiments, the CDR-L2 is a CDR-L2 of a VL domain of SEQ ID NO: 21, with up to 1, 2, 3, 4, 5, 6, 7, or 8 amino acid substitutions. In some embodiments, the CDR-L3 is a CDR-L3 of a VL domain of SEQ ID NO: 21, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises one to three CDRs of a VH domain selected from SEQ ID NOs: 7, 8, 9, 10, 11, 12, 13, 14, 15, 16, 17, 18, 19, and 20 and one to three CDRs of a VL domain of SEQ ID NO: 21. In some embodiments, an antibody provided herein comprises two to three CDRs of a VH domain selected from SEQ ID NOs: 7, 8, 9, 10, 11, 12, 13, 14, 15, 16, 17, 18, 19, and 20 and two to three CDRs of a VL domain of SEQ ID NO: 21. In some embodiments, an antibody provided herein comprises three CDRs of a VH domain selected from SEQ ID NOs: 7, 8, 9, 10, 11, 12, 13, 14, 15, 16, 17, 18, 19, and 20 and three CDRs of a VL domain of SEQ ID NO: 21. In some aspects, the CDRs are Exemplary CDRs. In some aspects, the CDRs are Kabat CDRs. In some aspects, the CDRs are Chothia CDRs. In some aspects, the CDRs are AbM CDRs. In some aspects, the CDRs are Contact CDRs. In some aspects, the CDRs are IMGT CDRs.

In some embodiments, an antibody provided herein comprises a CDR-H3 selected of SEQ ID NOs: 24, 25, 26, 27, 28, 29 and 30. In some aspects, the CDR-H3 has at least about 50%, 75%, 80%, 85%, 90%, or 95% identity with a CDR-H3 of SEQ ID NOs: 24, 25, 26, 27, 28, 29 and 30. In some embodiments, the CDR-H3 is a CDR-H3 selected of SEQ ID NO: 24, 25, 26, 27, 28, 29 and 30, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 selected of SEQ ID NO: 24. In some aspects, the CDR-H3 has at least about 50%, 75%, 80%, 85%, 90%, or 95% identity with a CDR-H3 of SEQ ID NO: 24. In some embodiments, the CDR-H3 is a CDR-H3 selected of SEQ ID NO: 24, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 selected of SEQ ID NO: 25. In some aspects, the CDR-H3 has at least about 50%, 75%, 80%, 85%, 90%, or 95% identity with a CDR-H3 of SEQ ID NO: 25. In some embodiments, the CDR-H3 is a CDR-H3 selected of SEQ ID NO: 25, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 selected of SEQ ID NO: 26. In some aspects, the CDR-H3 has at least about 50%, 75%, 80%, 85%, 90%, or 95% identity with a CDR-H3 of SEQ ID NO: 26. In some embodiments, the CDR-H3 is a CDR-H3 selected of SEQ ID NO: 26, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 selected of SEQ ID NO: 27. In some aspects, the CDR-H3 has at least about 50%, 75%, 80%, 85%, 90%, or 95% identity with a CDR-H3 of SEQ ID NO: 27. In some embodiments, the CDR-H3 is a CDR-H3 selected of SEQ ID NO: 27, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 selected of SEQ ID NO: 28. In some aspects, the CDR-H3 has at least about 50%, 75%, 80%, 85%, 90%, or 95% identity with a CDR-H3 of SEQ ID NO: 28. In some embodiments, the CDR-H3 is a CDR-H3 selected of SEQ ID NO: 28, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 selected of SEQ ID NO: 29. In some aspects, the CDR-H3 has at least about 50%, 75%, 80%, 85%, 90%, or 95% identity with a CDR-H3 of SEQ ID NO: 29. In some embodiments, the CDR-H3 is a CDR-H3 selected of SEQ ID NO: 29, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 selected of SEQ ID NO: 30. In some aspects, the CDR-H3 has at least about 50%, 75%, 80%, 85%, 90%, or 95% identity with a CDR-H3 of SEQ ID NO: 30. In some embodiments, the CDR-H3 is a CDR-H3 selected of SEQ ID NO: 30, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 selected of SEQ ID NO: 3. In some aspects, the CDR-H3 has at least about 50%, 75%, 80%, 85%, 90%, or 95% identity with a CDR-H3 of SEQ ID NO: 3. In some embodiments, the CDR-H3 is a CDR-H3 selected of SEQ ID NO: 3, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H1 of SEQ ID NO: 1. In some aspects, the CDR-H1 has at least about 50%, 75%, 80%, 85%, 90%, or 95% identity with a CDR-H1 of SEQ ID NO: 1. In some embodiments, the CDR-H1 is a CDR-H1 of SEQ ID NO: 1, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H2 selected of SEQ ID NO: 2. In some aspects, the CDR-H2 has at least about 50%, 75%, 80%, 85%, 90%, or 95% identity with a CDR-H2 of SEQ ID NO: 2. In some embodiments, the CDR-H2 is a CDR-H2 selected of SEQ ID NO: 2, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 of SEQ ID NO: 24 and a CDR-H2 of SEQ ID NO: 2. In some embodiments, an antibody provided herein comprises a CDR-H3 of SEQ ID NO: 25, a CDR-H2 of SEQ ID NO: 2, and a CDR-H1 of SEQ ID NO: 1. In some embodiments, the CDR-H3 has at least about 50%, 75%, 80%, 85%, 90%, or 95% identity with a CDR-H3 of SEQ ID NO: 24, the CDR-H2 has at least about 50%, 75%, 80%, 85%, 90%, or 95% identity with a CDR-H2 of SEQ ID NO: 2, and the CDR-H1 has at least about 50%, 75%, 80%, 85%, 90%, or 95% identity with a CDR-H1 of SEQ ID NO: 1. In some embodiments, the CDR-H3 is a CDR-H3 of SEQ ID NO: 24, with up to 1, 2, 3, 4, 5, 6, 7, or 8 amino acid substitutions; the CDR-H2 is a CDR-H2 of SEQ ID NO: 2, with up to 1, 2, 3, 4, 5, 6, 7, or 8 amino acid substitutions; and the CDR-H1 is a CDR-H1 of SEQ ID NO: 1, with up to 1, 2, 3, 4, or 5 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 of SEQ ID NO: 25 and a CDR-H2 of SEQ ID NO: 2. In some embodiments, an antibody provided herein comprises a CDR-H3 of SEQ ID NO: 25, a CDR-H2 of SEQ ID NO: 2, and a CDR-H1 of SEQ ID NO: 1. In some embodiments, the CDR-H3 has at least about 50%, 75%, 80%, 85%, 90%, or 95% identity with a CDR-H3 of SEQ ID NO: 25, the CDR-H2 has at least about 50%, 75%, 80%, 85%, 90%, or 95% identity with a CDR-H2 of SEQ ID NO: 2, and the CDR-H1 has at least about 50%, 75%, 80%, 85%, 90%, or 95% identity with a CDR-H1 of SEQ ID NO: 1. In some embodiments, the CDR-H3 is a CDR-H3 of SEQ ID NO: 25, with up to 1, 2, 3, 4, 5, 6, 7, or 8 amino acid substitutions; the CDR-H2 is a CDR-H2 of SEQ ID NO: 2, with up to 1, 2, 3, 4, 5, 6, 7, or 8 amino acid substitutions; and the CDR-H1 is a CDR-H1 of SEQ ID NO: 1, with up to 1, 2, 3, 4, or 5 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 of SEQ ID NO: 26 and a CDR-H2 of SEQ ID NO: 2. In some embodiments, an antibody provided herein comprises a CDR-H3 of SEQ ID NO: 26, a CDR-H2 of SEQ ID NO: 2, and a CDR-H1 of SEQ ID NO: 1. In some embodiments, the CDR-H3 has at least about 50%, 75%, 80%, 85%, 90%, or 95% identity with a CDR-H3 of SEQ ID NO: 26, the CDR-H2 has at least about 50%, 75%, 80%, 85%, 90%, or 95% identity with a CDR-H2 of SEQ ID NO: 2, and the CDR-H1 has at least about 50%, 75%, 80%, 85%, 90%, or 95% identity with a CDR-H1 of SEQ ID NO: 1. In some embodiments, the CDR-H3 is a CDR-H3 of SEQ ID NO: 26, with up to 1, 2, 3, 4, 5, 6, 7, or 8 amino acid substitutions; the CDR-H2 is a CDR-H2 of SEQ ID NO: 2, with up to 1, 2, 3, 4, 5, 6, 7, or 8 amino acid substitutions; and the CDR-H1 is a CDR-H1 of SEQ ID NO: 1, with up to 1, 2, 3, 4, or 5 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 of SEQ ID NO: 27 and a CDR-H2 of SEQ ID NO: 2. In some embodiments, an antibody provided herein comprises a CDR-H3 of SEQ ID NO: 27, a CDR-H2 of SEQ ID NO: 2, and a CDR-H1 of SEQ ID NO: 1. In some embodiments, the CDR-H3 has at least about 50%, 75%, 80%, 85%, 90%, or 95% identity with a CDR-H3 of SEQ ID NO: 27, the CDR-H2 has at least about 50%, 75%, 80%, 85%, 90%, or 95% identity with a CDR-H2 of SEQ ID NO: 2, and the CDR-H1 has at least about 50%, 75%, 80%, 85%, 90%, or 95% identity with a CDR-H1 of SEQ ID NO: 1. In some embodiments, the CDR-H3 is a CDR-H3 of SEQ ID NO: 27, with up to 1, 2, 3, 4, 5, 6, 7, or 8 amino acid substitutions; the CDR-H2 is a CDR-H2 of SEQ ID NO: 2, with up to 1, 2, 3, 4, 5, 6, 7, or 8 amino acid substitutions; and the CDR-H1 is a CDR-H1 of SEQ ID NO: 1, with up to 1, 2, 3, 4, or 5 amino acid substitutions. In some aspects, the amino acid substitutions are conservative amino acid substitutions. In some embodiments, the antibody described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 of SEQ ID NO: 28 and a CDR-H2 of SEQ ID NO: 2. In some embodiments, an antibody provided herein comprises a CDR-H3 of SEQ ID NO: 28, a CDR-H2 of SEQ ID NO: 2, and a CDR-H1 of SEQ ID NO: 1. In some embodiments, the CDR-H3 has at least about 50%, 75%, 80%, 85%, 90%, or 95% identity with a CDR-H3 of SEQ ID NO: 28, the CDR-H2 has at least about 50%, 75%, 80%, 85%, 90%, or 95% identity with a CDR-H2 of SEQ ID NO: 2, and the CDR-H1 has at least about 50%, 75%, 80%, 85%, 90%, or 95% identity with a CDR-H1 of SEQ ID NO: 1. In some embodiments, the CDR-H3 is a CDR-H3 of SEQ ID NO: 28, with up to 1, 2, 3, 4, 5, 6, 7, or 8 amino acid substitutions; the CDR-H2 is a CDR-H2 of SEQ ID NO: 2, with up to 1, 2, 3, 4, 5, 6, 7, or 8 amino acid substitutions; and the CDR-H1 is a CDR-H1 of SEQ ID NO: 1, with up to 1, 2, 3, 4, or 5 amino acid substitutions. In some aspects, the amino acid substitutions are conservative amino acid substitutions. In some embodiments, the antibody described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 of SEQ ID NO: 29 and a CDR-H2 of SEQ ID NO: 2. In some embodiments, an antibody provided herein comprises a CDR-H3 of SEQ ID NO: 29, a CDR-H2 of SEQ ID NO: 2, and a CDR-H1 of SEQ ID NO: 1. In some embodiments, the CDR-H3 has at least about 50%, 75%, 80%, 85%, 90%, or 95% identity with a CDR-H3 of SEQ ID NO: 29, the CDR-H2 has at least about 50%, 75%, 80%, 85%, 90%, or 95% identity with a CDR-H2 of SEQ ID NO: 2, and the CDR-H1 has at least about 50%, 75%, 80%, 85%, 90%, or 95% identity with a CDR-H1 of SEQ ID NO: 1. In some embodiments, the CDR-H3 is a CDR-H3 of SEQ ID NO: 29, with up to 1, 2, 3, 4, 5, 6, 7, or 8 amino acid substitutions; the CDR-H2 is a CDR-H2 of SEQ ID NO: 2, with up to 1, 2, 3, 4, 5, 6, 7, or 8 amino acid substitutions; and the CDR-H1 is a CDR-H1 of SEQ ID NO: 1, with up to 1, 2, 3, 4, or 5 amino acid substitutions. In some aspects, the amino acid substitutions are conservative amino acid substitutions. In some embodiments, the antibody described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 of SEQ ID NO: 30 and a CDR-H2 of SEQ ID NO: 2. In some embodiments, an antibody provided herein comprises a CDR-H3 of SEQ ID NO: 30, a CDR-H2 of SEQ ID NO: 2, and a CDR-H1 of SEQ ID NO: 1. In some embodiments, the CDR-H3 has at least about 50%, 75%, 80%, 85%, 90%, or 95% identity with a CDR-H3 of SEQ ID NO: 30, the CDR-H2 has at least about 50%, 75%, 80%, 85%, 90%, or 95% identity with a CDR-H2 of SEQ ID NO: 2, and the CDR-H1 has at least about 50%, 75%, 80%, 85%, 90%, or 95% identity with a CDR-H1 of SEQ ID NO: 1. In some embodiments, the CDR-H3 is a CDR-H3 of SEQ ID NO: 30, with up to 1, 2, 3, 4, 5, 6, 7, or 8 amino acid substitutions; the CDR-H2 is a CDR-H2 of SEQ ID NO: 2, with up to 1, 2, 3, 4, 5, 6, 7, or 8 amino acid substitutions; and the CDR-H1 is a CDR-H1 of SEQ ID NO: 1, with up to 1, 2, 3, 4, or 5 amino acid substitutions. In some aspects, the amino acid substitutions are conservative amino acid substitutions. In some embodiments, the antibody described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-L3 of SEQ ID NO: 6. In some aspects, the CDR-L3 has at least about 50%, 75%, 80%, 85%, 90%, or 95% identity with a CDR-L3 of SEQ ID NO: 6. In some embodiments, the CDR-L3 is a CDR-L3 of SEQ ID NO: 6, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-L2 of SEQ ID NO: 5. In some aspects, the CDR-L2 has at least about 50%, 75%, 80%, 85%, 90%, or 95% identity with a CDR-L2 of SEQ ID NO: 5. In some embodiments, the CDR-L2 is a CDR-L2 of SEQ ID NO: 5, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-L1 of SEQ ID NO: 4. In some aspects, the CDR-L1 has at least about 50%, 75%, 80%, 85%, 90%, or 95% identity with a CDR-L1 of SEQ ID NO: 4. In some embodiments, the CDR-L1 is a CDR-L1 of SEQ ID NO: 4, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-L3 of SEQ ID NO: 6 and a CDR-L2 of SEQ ID NO: 5. In some embodiments, an antibody provided herein comprises a CDR-L3 of SEQ ID NO: 6, a CDR-L2 of SEQ ID NO: 5, and a CDR-L1 of SEQ ID NO: 4. In some embodiments, the CDR-L3 has at least about 50%, 75%, 80%, 85%, 90%, or 95% identity with a CDR-L3 of SEQ ID NO: 6, the CDR-L2 has at least about 50%, 75%, 80%, 85%, 90%, or 95% identity with a CDR-L2 of SEQ ID NO: 5, and the CDR-L1 has at least about 50%, 75%, 80%, 85%, 90%, or 95% identity with a CDR-L1 of SEQ ID NO: 4. In some embodiments, the CDR-L3 is a CDR-L3 of SEQ ID NO: 6, with up to 1, 2, 3, 4, or 5 amino acid substitutions; the CDR-L2 is a CDR-L2 of SEQ ID NO: 5, with up to 1, 2, 3, or 4 amino acid substitutions; and the CDR-L1 is a CDR-L1 of SEQ ID NO: 4, with up to 1, 2, 3, 4, 5, or 6 amino acid substitutions. In some aspects, the amino acid substitutions are conservative amino acid substitutions. In some embodiments, the antibodies described herein are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 of SEQ ID NO: 24, a CDR-H2 of SEQ ID NO: 2, a CDR-H1 of SEQ ID NO: 1, a CDR-L3 of SEQ ID NO: 6, a CDR-L2 of SEQ ID NO: 5, and a CDR-L1 of SEQ ID NO: 4. In some embodiments, the CDR-H3 has at least about 50%, 75%, 80%, 85%, 90%, or 95% identity with a CDR-H3 of SEQ ID NO: 24, the CDR-H2 has at least about 50%, 75%, 80%, 85%, 90%, or 95% identity with a CDR-H2 of SEQ ID NO: 2, the CDR-H1 has at least about 50%, 75%, 80%, 85%, 90%, or 95% identity with a CDR-H1 of SEQ ID NO: 1, the CDR-L3 has at least about 50%, 75%, 80%, 85%, 90%, or 95% identity with a CDR-L3 of SEQ ID NO: 6, the CDR-L2 has at least about 50%, 75%, 80%, 85%, 90%, or 95% identity with a CDR-L2 of SEQ ID NO: 5, and the CDR-L1 has at least about 50%, 75%, 80%, 85%, 90%, or 95% identity with a CDR-L1 of SEQ ID NO: 4. In some embodiments, the CDR-H3 is a CDR-H3 of SEQ ID NO: 24, with up to 1, 2, 3, 4, 5, 6, 7, or 8 amino acid substitutions; the CDR-H2 is a CDR-H2 of SEQ ID NO: 2, with up to 1, 2, 3, 4, 5, 6, 7, or 8 amino acid substitutions; the CDR-H1 is a CDR-H1 of SEQ ID NO: 1, with up to 1, 2, 3, 4, or 5 amino acid substitutions; the CDR-L3 is a CDR-L3 of SEQ ID NO: 6, with up to 1, 2, 3, 4, or 5 amino acid substitutions; the CDR-L2 is a CDR-L2 of SEQ ID NO: 5, with up to 1, 2, 3, or 4 amino acid substitutions; and the CDR-L1 is a CDR-L1 of SEQ ID NO: 4, with up to 1, 2, 3, 4, 5, or 6 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 of SEQ ID NO: 25, a CDR-H2 of SEQ ID NO: 2, a CDR-H1 of SEQ ID NO: 1, a CDR-L3 of SEQ ID NO: 6, a CDR-L2 of SEQ ID NO: 5, and a CDR-L1 of SEQ ID NO: 4. In some embodiments, the CDR-H3 has at least about 50%, 75%, 80%, 85%, 90%, or 95% identity with a CDR-H3 of SEQ ID NO: 25, the CDR-H2 has at least about 50%, 75%, 80%, 85%, 90%, or 95% identity with a CDR-H2 of SEQ ID NO: 2, the CDR-H1 has at least about 50%, 75%, 80%, 85%, 90%, or 95% identity with a CDR-H1 of SEQ ID NO: 1, the CDR-L3 has at least about 50%, 75%, 80%, 85%, 90%, or 95% identity with a CDR-L3 of SEQ ID NO: 6, the CDR-L2 has at least about 50%, 75%, 80%, 85%, 90%, or 95% identity with a CDR-L2 of SEQ ID NO: 5, and the CDR-L1 has at least about 50%, 75%, 80%, 85%, 90%, or 95% identity with a CDR-L1 of SEQ ID NO: 4. In some embodiments, the CDR-H3 is a CDR-H3 of SEQ ID NO: 25, with up to 1, 2, 3, 4, 5, 6, 7, or 8 amino acid substitutions; the CDR-H2 is a CDR-H2 of SEQ ID NO: 2, with up to 1, 2, 3, 4, 5, 6, 7, or 8 amino acid substitutions; the CDR-H1 is a CDR-H1 of SEQ ID NO: 1, with up to 1, 2, 3, 4, or 5 amino acid substitutions; the CDR-L3 is a CDR-L3 of SEQ ID NO: 6, with up to 1, 2, 3, 4, or 5 amino acid substitutions; the CDR-L2 is a CDR-L2 of SEQ ID NO: 5, with up to 1, 2, 3, or 4 amino acid substitutions; and the CDR-L1 is a CDR-L1 of SEQ ID NO: 4, with up to 1, 2, 3, 4, 5, or 6 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 of SEQ ID NO: 26, a CDR-H2 of SEQ ID NO: 2, a CDR-H1 of SEQ ID NO: 1, a CDR-L3 of SEQ ID NO: 6, a CDR-L2 of SEQ ID NO: 5, and a CDR-L1 of SEQ ID NO: 4. In some embodiments, the CDR-H3 has at least about 50%, 75%, 80%, 85%, 90%, or 95% identity with a CDR-H3 of SEQ ID NO: 26, the CDR-H2 has at least about 50%, 75%, 80%, 85%, 90%, or 95% identity with a CDR-H2 of SEQ ID NO: 2, the CDR-H1 has at least about 50%, 75%, 80%, 85%, 90%, or 95% identity with a CDR-H1 of SEQ ID NO: 1, the CDR-L3 has at least about 50%, 75%, 80%, 85%, 90%, or 95% identity with a CDR-L3 of SEQ ID NO: 6, the CDR-L2 has at least about 50%, 75%, 80%, 85%, 90%, or 95% identity with a CDR-L2 of SEQ ID NO: 5, and the CDR-L1 has at least about 50%, 75%, 80%, 85%, 90%, or 95% identity with a CDR-L1 of SEQ ID NO: 4. In some embodiments, the CDR-H3 is a CDR-H3 of SEQ ID NO: 26, with up to 1, 2, 3, 4, 5, 6, 7, or 8 amino acid substitutions; the CDR-H2 is a CDR-H2 of SEQ ID NO: 2, with up to 1, 2, 3, 4, 5, 6, 7, or 8 amino acid substitutions; the CDR-H1 is a CDR-H1 of SEQ ID NO: 1, with up to 1, 2, 3, 4, or 5 amino acid substitutions; the CDR-L3 is a CDR-L3 of SEQ ID NO: 6, with up to 1, 2, 3, 4, or 5 amino acid substitutions; the CDR-L2 is a CDR-L2 of SEQ ID NO: 5, with up to 1, 2, 3, or 4 amino acid substitutions; and the CDR-L1 is a CDR-L1 of SEQ ID NO: 4, with up to 1, 2, 3, 4, 5, or 6 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 of SEQ ID NO: 27, a CDR-H2 of SEQ ID NO: 2, a CDR-H1 of SEQ ID NO: 1, a CDR-L3 of SEQ ID NO: 6, a CDR-L2 of SEQ ID NO: 5, and a CDR-L1 of SEQ ID NO: 4. In some embodiments, the CDR-H3 has at least about 50%, 75%, 80%, 85%, 90%, or 95% identity with a CDR-H3 of SEQ ID NO: 27, the CDR-H2 has at least about 50%, 75%, 80%, 85%, 90%, or 95% identity with a CDR-H2 of SEQ ID NO: 2, the CDR-H1 has at least about 50%, 75%, 80%, 85%, 90%, or 95% identity with a CDR-H1 of SEQ ID NO: 1, the CDR-L3 has at least about 50%, 75%, 80%, 85%, 90%, or 95% identity with a CDR-L3 of SEQ ID NO: 6, the CDR-L2 has at least about 50%, 75%, 80%, 85%, 90%, or 95% identity with a CDR-L2 of SEQ ID NO: 5, and the CDR-L1 has at least about 50%, 75%, 80%, 85%, 90%, or 95% identity with a CDR-L1 of SEQ ID NO: 4. In some embodiments, the CDR-H3 is a CDR-H3 of SEQ ID NO: 27, with up to 1, 2, 3, 4, 5, 6, 7, or 8 amino acid substitutions; the CDR-H2 is a CDR-H2 of SEQ ID NO: 2, with up to 1, 2, 3, 4, 5, 6, 7, or 8 amino acid substitutions; the CDR-H1 is a CDR-H1 of SEQ ID NO: 1, with up to 1, 2, 3, 4, or 5 amino acid substitutions; the CDR-L3 is a CDR-L3 of SEQ ID NO: 6, with up to 1, 2, 3, 4, or 5 amino acid substitutions; the CDR-L2 is a CDR-L2 of SEQ ID NO: 5, with up to 1, 2, 3, or 4 amino acid substitutions; and the CDR-L1 is a CDR-L1 of SEQ ID NO: 4, with up to 1, 2, 3, 4, 5, or 6 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 of SEQ ID NO: 28, a CDR-H2 of SEQ ID NO: 2, a CDR-H1 of SEQ ID NO: 1, a CDR-L3 of SEQ ID NO: 6, a CDR-L2 of SEQ ID NO: 5, and a CDR-L1 of SEQ ID NO: 4. In some embodiments, the CDR-H3 has at least about 50%, 75%, 80%, 85%, 90%, or 95% identity with a CDR-H3 of SEQ ID NO: 28, the CDR-H2 has at least about 50%, 75%, 80%, 85%, 90%, or 95% identity with a CDR-H2 of SEQ ID NO: 2, the CDR-H1 has at least about 50%, 75%, 80%, 85%, 90%, or 95% identity with a CDR-H1 of SEQ ID NO: 1, the CDR-L3 has at least about 50%, 75%, 80%, 85%, 90%, or 95% identity with a CDR-L3 of SEQ ID NO: 6, the CDR-L2 has at least about 50%, 75%, 80%, 85%, 90%, or 95% identity with a CDR-L2 of SEQ ID NO: 5, and the CDR-L1 has at least about 50%, 75%, 80%, 85%, 90%, or 95% identity with a CDR-L1 of SEQ ID NO: 4. In some embodiments, the CDR-H3 is a CDR-H3 of SEQ ID NO: 28, with up to 1, 2, 3, 4, 5, 6, 7, or 8 amino acid substitutions; the CDR-H2 is a CDR-H2 of SEQ ID NO: 2, with up to 1, 2, 3, 4, 5, 6, 7, or 8 amino acid substitutions; the CDR-H1 is a CDR-H1 of SEQ ID NO: 1, with up to 1, 2, 3, 4, or 5 amino acid substitutions; the CDR-L3 is a CDR-L3 of SEQ ID NO: 6, with up to 1, 2, 3, 4, or 5 amino acid substitutions; the CDR-L2 is a CDR-L2 of SEQ ID NO: 5, with up to 1, 2, 3, or 4 amino acid substitutions; and the CDR-L1 is a CDR-L1 of SEQ ID NO: 4, with up to 1, 2, 3, 4, 5, or 6 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 of SEQ ID NO: 29, a CDR-H2 of SEQ ID NO: 2, a CDR-H1 of SEQ ID NO: 1, a CDR-L3 of SEQ ID NO: 6, a CDR-L2 of SEQ ID NO: 5, and a CDR-L1 of SEQ ID NO: 4. In some embodiments, the CDR-H3 has at least about 50%, 75%, 80%, 85%, 90%, or 95% identity with a CDR-H3 of SEQ ID NO: 29, the CDR-H2 has at least about 50%, 75%, 80%, 85%, 90%, or 95% identity with a CDR-H2 of SEQ ID NO: 2, the CDR-H1 has at least about 50%, 75%, 80%, 85%, 90%, or 95% identity with a CDR-H1 of SEQ ID NO: 1, the CDR-L3 has at least about 50%, 75%, 80%, 85%, 90%, or 95% identity with a CDR-L3 of SEQ ID NO: 6, the CDR-L2 has at least about 50%, 75%, 80%, 85%, 90%, or 95% identity with a CDR-L2 of SEQ ID NO: 5, and the CDR-L1 has at least about 50%, 75%, 80%, 85%, 90%, or 95% identity with a CDR-L1 of SEQ ID NO: 4. In some embodiments, the CDR-H3 is a CDR-H3 of SEQ ID NO: 29, with up to 1, 2, 3, 4, 5, 6, 7, or 8 amino acid substitutions; the CDR-H2 is a CDR-H2 of SEQ ID NO: 2, with up to 1, 2, 3, 4, 5, 6, 7, or 8 amino acid substitutions; the CDR-H1 is a CDR-H1 of SEQ ID NO: 1, with up to 1, 2, 3, 4, or 5 amino acid substitutions; the CDR-L3 is a CDR-L3 of SEQ ID NO: 6, with up to 1, 2, 3, 4, or 5 amino acid substitutions; the CDR-L2 is a CDR-L2 of SEQ ID NO: 5, with up to 1, 2, 3, or 4 amino acid substitutions; and the CDR-L1 is a CDR-L1 of SEQ ID NO: 4, with up to 1, 2, 3, 4, 5, or 6 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 of SEQ ID NO: 30, a CDR-H2 of SEQ ID NO: 2, a CDR-H1 of SEQ ID NO: 1, a CDR-L3 of SEQ ID NO: 6, a CDR-L2 of SEQ ID NO: 5, and a CDR-L1 of SEQ ID NO: 4. In some embodiments, the CDR-H3 has at least about 50%, 75%, 80%, 85%, 90%, or 95% identity with a CDR-H3 of SEQ ID NO: 30, the CDR-H2 has at least about 50%, 75%, 80%, 85%, 90%, or 95% identity with a CDR-H2 of SEQ ID NO: 2, the CDR-H1 has at least about 50%, 75%, 80%, 85%, 90%, or 95% identity with a CDR-H1 of SEQ ID NO: 1, the CDR-L3 has at least about 50%, 75%, 80%, 85%, 90%, or 95% identity with a CDR-L3 of SEQ ID NO: 6, the CDR-L2 has at least about 50%, 75%, 80%, 85%, 90%, or 95% identity with a CDR-L2 of SEQ ID NO: 5, and the CDR-L1 has at least about 50%, 75%, 80%, 85%, 90%, or 95% identity with a CDR-L1 of SEQ ID NO: 4. In some embodiments, the CDR-H3 is a CDR-H3 of SEQ ID NO: 30, with up to 1, 2, 3, 4, 5, 6, 7, or 8 amino acid substitutions; the CDR-H2 is a CDR-H2 of SEQ ID NO: 2, with up to 1, 2, 3, 4, 5, 6, 7, or 8 amino acid substitutions; the CDR-H1 is a CDR-H1 of SEQ ID NO: 1, with up to 1, 2, 3, 4, or 5 amino acid substitutions; the CDR-L3 is a CDR-L3 of SEQ ID NO: 6, with up to 1, 2, 3, 4, or 5 amino acid substitutions; the CDR-L2 is a CDR-L2 of SEQ ID NO: 5, with up to 1, 2, 3, or 4 amino acid substitutions; and the CDR-L1 is a CDR-L1 of SEQ ID NO: 4, with up to 1, 2, 3, 4, 5, or 6 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H1 of SEQ ID NO: 1, a CDR-H2 of SEQ ID NO: 2, a CDR-H3 of SEQ ID NO: 24, a CDR-L1 of SEQ ID NO: 4, a CDR-L2 of SEQ ID NO: 5, and a CDR-L3 of SEQ ID NO: 6.

In some embodiments, an antibody provided herein comprises a CDR-H1 of SEQ ID NO: 1, a CDR-H2 of SEQ ID NO: 2, a CDR-H3 of SEQ ID NO: 25, a CDR-L1 of SEQ ID NO: 4, a CDR-L2 of SEQ ID NO: 5, and a CDR-L3 of SEQ ID NO: 6.

In some embodiments, an antibody provided herein comprises a CDR-H1 of SEQ ID NO: 1, a CDR-H2 of SEQ ID NO: 2, a CDR-H3 of SEQ ID NO: 26, a CDR-L1 of SEQ ID NO: 4, a CDR-L2 of SEQ ID NO: 5, and a CDR-L3 of SEQ ID NO: 6.

In some embodiments, an antibody provided herein comprises a CDR-H1 of SEQ ID NO: 1, a CDR-H2 of SEQ ID NO: 2, a CDR-H3 of SEQ ID NO: 27, a CDR-L1 of SEQ ID NO: 4, a CDR-L2 of SEQ ID NO: 5, and a CDR-L3 of SEQ ID NO: 6.

In some embodiments, an antibody provided herein comprises a CDR-H1 of SEQ ID NO: 1, a CDR-H2 of SEQ ID NO: 2, a CDR-H3 of SEQ ID NO: 28, a CDR-L1 of SEQ ID NO: 4, a CDR-L2 of SEQ ID NO: 5, and a CDR-L3 of SEQ ID NO: 6.

In some embodiments, an antibody provided herein comprises a CDR-H1 of SEQ ID NO: 1, a CDR-H2 of SEQ ID NO: 2, a CDR-H3 of SEQ ID NO: 29, a CDR-L1 of SEQ ID NO: 4, a CDR-L2 of SEQ ID NO: 5, and a CDR-L3 of SEQ ID NO: 6.

In some embodiments, an antibody provided herein comprises a CDR-H1 of SEQ ID NO: 1, a CDR-H2 of SEQ ID NO: 2, a CDR-H3 of SEQ ID NO: 30, a CDR-L1 of SEQ ID NO: 4, a CDR-L2 of SEQ ID NO: 5, and a CDR-L3 of SEQ ID NO: 6.

### Fc Region

The structures of the Fc regions of various immunoglobulins, and the glycosylation sites contained therein, are known in the art. See Schroeder and Cavacini, J. Allergy Clin. Immunol., 2010, 125:S41-52, incorporated by reference in its entirety. The Fc region may be a naturally occurring Fc region, or an Fc region modified as described in the art or elsewhere in this disclosure.

Unless otherwise specified herein, numbering of amino acid residues in the Fc region or constant region is according to the EU numbering system, also called the EU index, as described in Kabat et al, Sequences of Proteins of Immunological Interest, 5th Ed. Public Health Service, National Institutes of Health, Bethesda, MD, 1991. An “Fc polypeptide” of a dimeric Fc as used herein refers to one of the two polypeptides forming the dimeric Fc domain, i.e. a polypeptide comprising C-terminal constant regions of an immunoglobulin heavy chain, capable of stable self-association. For example, an Fc polypeptide of a dimeric IgG Fc comprises an IgG CH2 and an IgG CH3 constant domain sequence. An Fc can be of the class IgA, IgD, IgE, IgG, and IgM, and several of these may be further divided into subclasses (isotypes), e.g., IgG₁, IgG₂, IgG₃, IgG₄, IgA₁, and IgA₂.

The terms “Fc receptor” and “FcR” are used to describe a receptor that binds to the Fc region of an antibody. For example, an FcR can be a native sequence human FcR. Generally, an FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Immunoglobulins of other isotypes can also be bound by certain FcRs (see, e.g., Janeway et al., Immuno Biology: the immune system in health and disease, (Elsevier Science Ltd., NY) (4th ed., 1999)). Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (reviewed in Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976); and Kim et al., J. Immunol. 24:249 (1994)).

Modifications in the CH2 domain can affect the binding of FcRs to the Fc. A number of amino acid modifications in the Fc region are known in the art for selectively altering the affinity of the Fc for different Fcgamma receptors. In some aspects, the Fc comprises one or more modifications to promote selective binding of Fc-gamma receptors.

Exemplary mutations that alter the binding of FcRs to the Fc are listed below:

S298A/E333A/K334A, S298A/E333A/K334A/K326A (Lu Y, Vernes J M, Chiang N, et al. J Immunol Methods. 2011 Feb. 28; 365 (1-2):132-41);

F243L/R292P/Y300L/V305I/P396L, F243L/R292P/Y300L/L235V/P396L (Stavenhagen J B, Gorlatov S, Tuaillon N, et al. Cancer Res. 2007 Sep. 15; 67(18):8882-90; Nordstrom J L, Gorlatov S, Zhang W, et al. Breast Cancer Res. 2011 Nov. 30; 13 (6):R123);

F243L (Stewart R, Thom G, Levens M, et al. Protein Eng Des Sel. 2011 September; 24(9):671-8), S298A/E333A/K334A (Shields R L, Namenuk A K, Hong K, et al. J Biol Chem. 2001 Mar. 2; 276(9):6591-604);
S239D/1332E/A330L, S239D/1332E (Lazar G A, Dang W, Karki S, et al. Proc Natl Acad Sci USA. 2006 Mar. 14; 103(11):4005-10);

S239D/S267E, S267E/L328F (Chu S Y, Vostiar I, Karki S, et al. Mol Immunol. 2008 September; 45(15):3926-33);

S239D/D265S/S298A/1332E, S239E/S298A/K326A/A327H, G237F/S298A/A330 L/1332E, S239D/1332E/S298A, S239D/K326E/A330L/1332E/S298A, G236A/S239D/D270L/1332E, S239E/S267E/H268D, L234F/S267E/N325L, G237F/V266L/S267D and other mutations listed in WO2011/120134 and WO2011/120135, herein incorporated by reference. Therapeutic Antibody Engineering (by William R. Strohl and Lila M. Strohl, Woodhead Publishing series in Biomedicine No 11, ISBN 1 907568 37 9, October 2012) lists mutations on page 283.

In some embodiments an antibody described herein includes modifications to improve its ability to mediate effector function. Such modifications are known in the art and include afucosylation, or engineering of the affinity of the Fc towards an activating receptor, mainly FCGR3a for ADCC, and towards C1q for CDC. The following Table B summarizes various designs reported in the literature for effector function engineering.

Methods of producing antibodies with little or no fucose on the Fc glycosylation site (Asn 297 EU numbering) without altering the amino acid sequence are well known in the art. The GlymaX® technology (ProBioGen AG) is based on the introduction of a gene for an enzyme which deflects the cellular pathway of fucose biosynthesis into cells used for antibody production. This prevents the addition of the sugar “fucose” to the N-linked antibody carbohydrate part by antibody-producing cells. (von Horsten et al. (2010) Glycobiology. 2010 December; 20 (12): 1607-18. Another approach to obtaining antibodies with lowered levels of fucosylation can be found in U.S. Pat. No. 8,409,572, which teaches selecting cell lines for antibody production for their ability to yield lower levels of fucosylation on antibodies can be fully afucosylated (meaning they contain no detectable fucose) or they can be partially afucosylated, meaning that the isolated antibody contains less than 95%, less than 85%, less than 75%, less than 65%, less than 55%, less than 45%, less than 35%, less than 25%, less than 15% or less than 5% of the amount of fucose normally detected for a similar antibody produced by a mammalian expression system.

Thus, in one embodiment, an antibody described herein can include a dimeric Fc that comprises one or more amino acid modifications as noted in Table B that confer improved effector function. In another embodiment, the antibody can be afucosylated to improve effector function.

TABLE B
CH2 domains and effector function engineering
Table B

| Reference | Mutations | Effect |
|---|---|---|
| Lu, 2011, Ferrara 2011, | Afucosylated | Increased ADCC |
| Mizushima 2011 | | |
| Lu, 2011 | S298A/E333A/K334A | Increased ADCC |
| Lu, 2011 | S298A/E333A/K334A/K326A | Increased ADCC |
| Stavenhagen, 2007 | F243L/R292P/Y300L/V305I/P396L | Increased ADCC |
| Nordstrom, 2011 | F243L/R292P/Y300L/L235V/P396L | Increased ADCC |
| Stewart, 2011 | F243L | Increased ADCC |
| Shields, 2001 | S298A/E333A/K334A | Increased ADCC |
| Lazar, 2006 | S239D/1332E/A330L | Increased ADCC |
| Lazar, 2006 | S239D/1332E | Increased ADCC |
| Bowles, 2006 | AME-D, not specified mutations | Increased ADCC |
| Heider, 2011 | 37.1, mutations not disclosed | Increased ADCC |
| Moore, 2010 | S267E/H268F/S324T | Increased CDC |

Fc modifications reducing FcgR and/or complement binding and/or effector function are known in the art. Recent publications describe strategies that have been used to engineer antibodies with reduced or silenced effector activity (see Strohl, WR (2009), Curr Opin Biotech 20:685-691, and Strohl, WR and Strohl LM, “Antibody Fc engineering for optimal antibody performance” In Therapeutic Antibody Engineering, Cambridge: Woodhead Publishing (2012), pp 225-249). These strategies include reduction of effector function through modification of glycosylation, use of IgG2/IgG4 scaffolds, or the introduction of mutations in the hinge or CH2 regions of the Fc. For example, US Patent Publication No. 2011/0212087 (Strohl), International Patent Publication No. WO 2006/105338 (Xencor), US Patent Publication No. 2012/0225058 (Xencor), US Patent Publication No. 2012/0251531 (Genentech), and Strop et al ((2012) J. Mol. Biol. 420: 204-219) describe specific modifications to reduce FcgR or complement binding to the Fc.

Specific, non-limiting examples of known amino acid modifications to reduce FcgR or complement binding to the Fc include those identified in the following Table C:

TABLE C
Modifications to reduce FcgR or complement binding to the Fc
Table C

| Company | Mutations |
|---|---|
| GSK | N297A |
| Ortho Biotech | L234A/L235A |
| Protein Design labs | IGG2 V234A/G237A |
| Wellcome Labs | IGG4 L235A/G237A/E318A |
| GSK | IGG4 S228P/L236E |
| Alexion | IGG2/IGG4combo |
| Merck | IGG2 H268Q/V309L/A330S/A331S |
| Bristol-Myers | C220S/C226S/C229S/P238S |
| Seattle Genetics | C226S/C229S/E3233P/L235V/L235A |
| Amgen | E. coli production, non glyco |
| Medimune | L234F/L235E/P331S |
| Trubion | Hinge mutant, possibly C226S/P230S |

Methods of producing antibodies with little or no fucose on the Fc glycosylation site (Asn 297 EU numbering) without altering the amino acid sequence are well known in the art. The GlymaxX® technology (ProBioGen AG) is based on the introduction of a gene for an enzyme which deflects the cellular pathway of fucose biosynthesis into cells used for antibody production. This prevents the addition of the sugar “fucose” to the N-linked antibody carbohydrate part by antibody-producing cells. (von Horsten et al. (2010) Glycobiology. 2010 December; 20 (12): 1607-18.) Examples of cell lines capable of producing defucosylated antibody include CHO-DG44 with stable overexpression of the bacterial oxidoreductase GDP-6-deoxy-D-lyxo-4-hexylose reductase (RMD) (see Henning von Horsten et al., Glycobiol 2010, 20:1607-1618) or Lec13 CHO cells, which are deficient in protein fucosylation (see Ripka et al., Arch. Biochem. Biophys., 1986, 249:533-545; U.S. Pat. Pub. No. 2003/0157108; WO 2004/056312; each of which is incorporated by reference in its entirety), and knockout cell lines, such as alpha-1,6-fucosyltransferase gene or FUT8 knockout CHO cells (see Yamane-Ohnuki et al., Biotech. Bioeng., 2004, 87: 614-622; Kanda et al., Biotechnol. Bioeng., 2006, 94:680-688; and WO 2003/085107; each of which is incorporated by reference in its entirety). Another approach to obtaining antibodies with lowered levels of fucosylation can be found in U.S. Pat. No. 8,409,572, which teaches selecting cell lines for antibody production for their ability to yield lower levels of fucosylation on antibodies

Examples of cell lines capable of producing defucosylated antibody include CHO-DG44 with stable overexpression of the bacterial oxidoreductase GDP-6-deoxy-D-lyxo-4-hexylose reductase (RMD) (see Henning von Horsten et al., Glycobiol 2010, 20:1607-1618) or Lec13 CHO cells, which are deficient in protein fucosylation (see Ripka et al., Arch. Biochem. Biophys., 1986, 249:533-545; U.S. Pat. Pub. No. 2003/0157108; WO 2004/056312; each of which is incorporated by reference in its entirety), and knockout cell lines, such as alpha-1,6-fucosyltransferase gene or FUT8 knockout CHO cells (see Yamane-Ohnuki et al., Biotech. Bioeng., 2004, 87: 614-622; Kanda et al., Biotechnol. Bioeng., 2006, 94:680-688; and WO 2003/085107; each of which is incorporated by reference in its entirety).

Antibodies can be fully afucosylated (meaning they contain no detectable fucose) or they can be partially afucosylated, meaning that the isolated antibody contains less than 95%, less than 85%, less than 75%, less than 65%, less than 55%, less than 45%, less than 35%, less than 25%, less than 15% or less than 5% of the amount of fucose normally detected for a similar antibody produced by a mammalian expression system.

In some aspects, an antibody provided herein comprises an IgG1 domain with reduced fucose content at position Asn 297 compared to a naturally occurring IgG1 domain. Such Fc domains are known to have improved ADCC. See Shields et al., J. Biol. Chem., 2002, 277:26733-26740, incorporated by reference in its entirety. In some aspects, such antibodies do not comprise any fucose at position Asn 297. The amount of fucose may be determined using any suitable method, for example as described in WO 2008/077546, incorporated by reference in its entirety.

In certain embodiments, an antibody provided herein comprises an Fc region with one or more amino acid substitutions which improve ADCC, such as a substitution at one or more of positions 298, 333, and 334 of the Fc region. In some embodiments, an antibody provided herein comprises an Fc region with one or more amino acid substitutions at positions 239, 332, and 330, as described in Lazar et al., Proc. Natl. Acad. Sci. USA, 2006, 103:4005-4010, incorporated by reference in its entirety.

Other illustrative glycosylation variants which may be incorporated into the antibodies provided herein are described, for example, in U.S. Pat. Pub. Nos. 2003/0157108, 2004/0093621, 2003/0157108, 2003/0115614, 2002/0164328, 2004/0093621, 2004/0132140, 2004/0110704, 2004/0110282, 2004/0109865; International Pat. Pub. Nos. 2000/61739, 2001/29246, 2003/085119, 2003/084570, 2005/035586, 2005/035778; 2005/053742, 2002/031140; Okazaki et al., J. Mol. Biol., 2004, 336:1239-1249; and Yamane-Ohnuki et al., Biotech. Bioeng., 2004, 87: 614-622; each of which is incorporated by reference in its entirety.

In some embodiments, an antibody provided herein comprises an Fc region with at least one galactose residue in the oligosaccharide attached to the Fc region. Such antibody variants may have improved CDC function. Examples of such antibody variants are described, for example, in WO 1997/30087; WO 1998/58964; and WO 1999/22764; each of which his incorporated by reference in its entirety.

In some embodiments, an antibody provided herein comprises one or more alterations that improves or diminishes C1q binding and/or CDC. See U.S. Pat. No. 6,194,551; WO 99/51642; and Idusogie et al., J. Immunol., 2000, 164:4178-4184; each of which is incorporated by reference in its entirety.

### Binding

The affinity of a molecule X for its partner Y can be represented by the dissociation equilibrium constant (K_{D}). The kinetic components that contribute to the dissociation equilibrium constant are described in more detail below. Affinity can be measured by common methods known in the art, including those described herein, such as surface plasmon resonance (SPR) technology (e.g., BIACORE®) or biolayer interferometry (e.g., FORTEBIO®).

With regard to the binding of an antibody to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the antibody to the target molecule is competitively inhibited by the control molecule. In some embodiments, the affinity of a fibrin antibody for a non-target molecule is less than about 50% of the affinity for fibrin. In some embodiments, the affinity of a fibrin antibody for a non-target molecule is less than about 40% of the affinity for fibrin. In some embodiments, the affinity of a fibrin antibody for a non-target molecule is less than about 30% of the affinity for fibrin. In some embodiments, the affinity of a fibrin antibody for a non-target molecule is less than about 20% of the affinity for fibrin. In some embodiments, the affinity of a fibrin antibody for a non-target molecule is less than about 10% of the affinity for fibrin. In some embodiments, the affinity of a fibrin antibody for a non-target molecule is less than about 1% of the affinity for fibrin. In some embodiments, the affinity of a fibrin antibody for a non-target molecule is less than about 0.1% of the affinity for fibrin.

When used herein in the context of two or more antibodies, the term “competes with” or “cross-competes with” indicates that the two or more antibodies compete for binding to an antigen (e.g., fibrin). In one exemplary assay, fibrin is coated on a surface and contacted with a first fibrin antibody, after which a second fibrin antibody is added. In another exemplary assay, a first fibrin antibody is coated on a surface and contacted with fibrin, and then a second fibrin antibody is added. If the presence of the first fibrin antibody reduces binding of the second fibrin antibody, in either assay, then the antibodies compete with each other. The term “competes with” also includes combinations of antibodies where one antibody reduces binding of another antibody, but where no competition is observed when the antibodies are added in the reverse order. However, in some embodiments, the first and second antibodies inhibit binding of each other, regardless of the order in which they are added. In some embodiments, one antibody reduces binding of another antibody to its antigen by at least 25%, at least 50%, at least 60%, at least 70%, at least 80%, at least 85%, at least 90%, at least 95%, or at least 99% as measured in a competitive binding assay. A skilled artisan can select the concentrations of the antibodies used in the competition assays based on the affinities of the antibodies for fibrin and the valency of the antibodies. The assays described in this definition are illustrative, and a skilled artisan can utilize any suitable assay to determine if antibodies compete with each other. Suitable assays are described, for example, in Cox et al., “Immunoassay Methods,” in Assay Guidance Manual [Internet], Updated Dec. 24, 2014 (ncbi.nlm.nih.gov/books/NBK92434/; accessed Sep. 29, 2015); Silman et al., Cytometry, 2001, 44:30-37; and Finco et al., J. Pharm. Biomed. Anal., 2011, 54:351-358; each of which is incorporated by reference in its entirety.

A test antibody competes with a reference antibody if an excess of a test antibody (e.g., at least 2×, 5×, 10×, 20×, or 100×) inhibits or blocks binding of the reference antibody by, e.g., at least 50%, 60%, 70%, 75%, 80%, 85%, 90%, 95%, or 99% as measured in a competitive binding assay. Antibodies identified by competition assay (competing antibody) include antibodies binding to the same epitope as the reference antibody and antibodies binding to an adjacent epitope sufficiently proximal to the epitope bound by the reference antibody for steric hindrance to occur. For example, a second, competing antibody can be identified that competes for binding to fibrin with a first antibody described herein. In certain instances, the second antibody can block or inhibit binding of the first antibody by, e.g., at least 50%, 60%, 70%, 75%, 80%, 85%, 90%, 95%, or 99% as measured in a competitive binding assay. In certain instances, the second antibody can displace the first antibody by greater than 50%, 60%, 70%, 75%, 80%, 85%, 90%, 95%, or 99%.

In some embodiments, an anti-fibrin antibody does not substantially bind myeloid cells present outside of cancer tissue. In some embodiments, an anti-fibrin antibody does not substantially bind stimulatory myeloid cells present in cancer tissue.

In some embodiments, an anti-fibrin antibody binds to residues γ377-395 of the fibrin or fibrinogen γC domain (SEQ ID NO: 31) of human fibrin. The binding epitope includes the residues within the numerical range (e.g., residues 377-395 of fibrin), the beginning residue of each range (e.g., residues 377-394 of human fibrin) and the end residue of each range (e.g., residues 378-395 of human fibrin), or any combination thereof.

In some embodiments, an antibody provided herein binds human Fibrin with a K_{D} of less than or equal to about 0.001, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 1.95, 2, 2.5, 3, 3.5, 4, 4.5, 5, 6, 7, 8, 9, or 10×10⁻⁶ M, as measured by Biacore assay. In some embodiments, the K_{D} of the antibody provided herein is between about 0.001-0.01, 0.01-0.1, 0.01-0.05, 0.05-0.1, 0.1-0.5, 0.5-1, 0.25-0.75, 0.25-0.5, 0.5-0.75, 0.75-1, 0.75-2, 1.1-1.2, 1.2-1.3, 1.3-1.4, 1.4-1.5, 1.5-1.6, 1.6-1.7, 1.7-1.8, 1.8-1.9, 1.9-2, 1-2, 1-5, 2-7, 3-8, 3-5, 4-6, 5-7, 6-8, 7-9, 7-10, or 5-10×10⁻⁶ M, as measured by Biacore assay. In some embodiments, an antibody provided herein binds human Fibrin with a K_{D} of less than or equal to about 1×10⁻⁵ M, 1×10⁻⁶ M, 1×10⁻⁷ M, 1×10⁻⁸ M, or 1×10⁻⁹ M.

In some embodiments, the antibody provided herein binds human fibrin with a K_{D} of less than or equal to about 10, 9, 8, 7, 6, 5, 4.5, 4, 3.5, 3, 2.5, 2, 1.98, 1.95, 1.9, 1.85, 1.8, 1.75, 1.7, 1.65, 1.6, 1.55, 1.50, 1.45, 1.4, 1.3, 1.2, 1.1, 1, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.01, 0.005, 0.001, 0.0005, or 0.0001×10⁻⁵ M, or less, as measured by Biacore assay. In some embodiments, the antibody provided herein binds human fibrin with a K_{D} between 5-3, 4-2, 3-1, 1.9-1.8, 1.8-1.7, 1.7-1.6, 1.6-1.5, 1.9-1.5, 1.5-1, 1-0.8, 1-0.5, 0.9-0.6, 0.7-0.4, 0.6-0.2, 0.5-0.3, 0.3-0.2, 0.2-0.1, 0.1-0.01, 0.01-0.001, or 0.001-0.0001×10⁻⁵ M as measured by Biacore assay. In some embodiments, the antibody provided herein binds human fibrin with a K_{d} of less than or equal to about 10, 9.56, 9.5, 9.0, 8.88, 8.84, 8.5, 8, 7.5, 7.32, 7, 6.5, 6, 5.5, 5, 4.5, 4, 3.5, 3, 2.5, 2, 1.5, or 1×10⁻⁴ (1/s), or less, as measured by Biacore assay. In some embodiments, the antibody provided herein binds human fibrin with a K_{d} between 7-10, 7-8, 8-9, 9-10, 7-7.5, 7.5-8, 8.-8.5, 8.5-9, 9-9,5, or 9.5-10×10⁻⁴ (1/s) as measured by Biacore assay. In some embodiments, the antibody provided herein binds human fibrin with a Kₐ of greater than or equal to about 4, 4.1, 4.2, 4.3, 4.4, 4.5, 4.6, 4.7, 4.8, 4.9, 45, 5.1, 5.2, 5.3, 5.4, 5.5, 5.6, 5.7, 5.8, 5.9, 6, 7, 8, 9, or 10×10⁵ (1/Ms), or more, as measured by Biacore assay. In some embodiments, the antibody provided herein binds human FIBRIN with a Kₐ between 4-7, 4-4.5, 4.5-5, 5-5.5, 5.5-6, 6-6.5, or 6.5-7, 7-8, 8-9, or 9-10×10⁵ (1/Ms) as measured by Biacore assay.

### Function

“Effector functions” refer to those biological activities mediated by the Fc region of an antibody, which activities may vary depending on the antibody isotype. Examples of antibody effector functions include receptor ligand blocking, agonism, or antagonism, C1q binding to activate complement dependent cytotoxicity (CDC), Fc receptor binding to activate antibody-dependent cellular cytotoxicity (ADCC), and antibody dependent cellular phagocytosis (ADCP). In some embodiments, the effector function of the fibrin antibody described herein is antagonism and blocks Mac-1 receptor binding to fibrin.

### Pharmaceutical Compositions

The present application provides compositions comprising the antibodies including pharmaceutical compositions comprising any one or more of the antibodies described herein with one or more pharmaceutically acceptable excipients. In some embodiments the composition is sterile. The pharmaceutical compositions generally comprise an effective amount of an antibody.

These compositions can comprise, in addition to one or more of the antibodies disclosed herein, a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives can be included, as required.

The anti-fibrin antibody that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

### Methods

### Methods of Preparation

Antibodies described herein can be produced using recombinant methods and compositions, e.g., as described in U.S. Pat. No. 4,816,567. In one embodiment, isolated nucleic acid encoding an antibody described herein is provided. Such nucleic acid may encode an amino acid sequence comprising the VL and/or an amino acid sequence comprising the VH of the antibody (e.g., the light and/or heavy chains of the antibody) or an amino acid sequence comprising the VHH of a single domain antibody. In a further embodiment, one or more vectors (e.g., expression vectors) comprising such nucleic acid are provided. In one embodiment, the nucleic acid is provided in a multicistronic vector. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antigen-binding polypeptide construct, or (2) a first vector comprising a nucleic acid that encodes an amino acid sequence comprising the VL of the antigen-binding polypeptide construct and a second vector comprising a nucleic acid that encodes an amino acid sequence comprising the VH of the antigen-binding polypeptide construct. In one embodiment, the host cell is eukaryotic, e.g. a Chinese Hamster Ovary (CHO) cell, or human embryonic kidney (HEK) cell, or lymphoid cell (e.g., Y0, NS0, Sp20 cell). In one embodiment, a method of making an antibody is provided, wherein the method comprises culturing a host cell comprising nucleic acid encoding the antibody, as provided above, under conditions suitable for expression of the antibody, and optionally recovering the antibody from the host cell (or host cell culture medium).

For recombinant production of the antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

When a heteromultimer or variant thereof is recombinantly produced by the host cells, the protein in certain embodiments is present at about 30%, about 25%, about 20%, about 15%, about 10%, about 5%, about 4%, about 3%, about 2%, or about 1% or less of the dry weight of the cells. When the heteromultimer or variant thereof is recombinantly produced by the host cells, the protein, in certain embodiments, is present in the culture medium at about 5 g/L, about 4 g/L, about 3 g/L, about 2 g/L, about 1 g/L, about 750 mg/L, about 500 mg/L, about 250 mg/L, about 100 mg/L, about 50 mg/L, about 10 mg/L, or about 1 mg/L or less of the dry weight of the cells. In certain embodiments, “substantially purified” heteromultimer produced by the methods described herein, has a purity level of at least about 30%, at least about 35%, at least about 40%, at least about 45%, at least about 50%, at least about 55%, at least about 60%, at least about 65%, at least about 70%, specifically, a purity level of at least about 75%, 80%, 85%, and more specifically, a purity level of at least about 90%, a purity level of at least about 95%, a purity level of at least about 99% or greater as determined by appropriate methods such as SDS/PAGE analysis, RP-HPLC, SEC, and capillary electrophoresis.

Suitable host cells for cloning or expression of antibody-encoding vectors include prokaryotic or eukaryotic cells described herein.

Recombinant host cells or host cells are cells that include an exogenous polynucleotide, regardless of the method used for insertion, for example, direct uptake, transduction, f-mating, or other methods known in the art to create recombinant host cells. The exogenous polynucleotide may be maintained as a nonintegrated vector, for example, a plasmid, or alternatively, may be integrated into the host genome. Host cells can include CHO, derivatives of CHO, NS0, Sp20, CV-1, VERO-76, HeLa, HepG2, Per.C6, or BHK.

For example, antibody may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibodies are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, N.J.), pp. 255-268 (2003).

In one embodiment, the antibodies described herein are produced in stable mammalian cells, by a method comprising: transfecting at least one stable mammalian cell with: nucleic acid encoding the antibody, in a predetermined ratio; and expressing the nucleic acid in the at least one mammalian cell. In some embodiments, the predetermined ratio of nucleic acid is determined in transient transfection experiments to determine the relative ratio of input nucleic acids that results in the highest percentage of the antibody in the expressed product.

In some embodiments, is the method of producing an antibody in stable mammalian cells as described herein wherein the expression product of the at least one stable mammalian cell comprises a larger percentage of the desired glycosylated antibody as compared to the monomeric heavy or light chain polypeptides, or other antibodies.

In some embodiments, is the method of producing a glycosylated antibody in stable mammalian cells described herein, said method comprising identifying and purifying the desired glycosylated antibody. In some embodiments, the said identification is by one or both of liquid chromatography and mass spectrometry.

If required, the antibodies can be purified or isolated after expression. Proteins may be isolated or purified in a variety of ways known to those skilled in the art. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques. Ultrafiltration and diafiltration techniques, in conjunction with protein concentration, are also useful. As is well known in the art, a variety of natural proteins bind Fc and antibodies, and these proteins can find use in the present invention for purification of antibodies. For example, the bacterial proteins A and G bind to the Fc region. Likewise, the bacterial protein L binds to the Fab region of some antibodies. Purification can often be enabled by a particular fusion partner. For example, antibodies may be purified using glutathione resin if a GST fusion is employed, Ni-2 affinity chromatography if a His-tag is employed or immobilized anti-flag antibody if a flag-tag is used. For general guidance in suitable purification techniques, see, e.g. incorporated entirely by reference Protein Purification: Principles and Practice, 3rd Ed., Scopes, Springer-Verlag, NY, 1994, incorporated entirely by reference. The degree of purification necessary will vary depending on the use of the antibodies. In some instances, no purification is necessary.

In certain embodiments, the antibodies are purified using Anion Exchange Chromatography including, but not limited to, chromatography on Q-sepharose, DEAE sepharose, poros HQ, poros DEAF, Toyopearl Q, Toyopearl QAE, Toyopearl DEAE, Resource/Source Q and DEAE, Fractogel Q and DEAE columns.

In specific embodiments, the proteins described herein are purified using Cation Exchange Chromatography including, but not limited to, SP-sepharose, CM sepharose, poros HS, poros C M, Toyopearl SP, Toyopearl CM, Resource/Source S and CM, Fractogel S and CM columns and their equivalents and comparables.

In addition, antibodies described herein can be chemically synthesized using techniques known in the art (e.g., see Creighton, 1983, Proteins: Structures and Molecular Principles, W. H. Freeman & Co., N.Y and Hunkapiller et al., Nature, 310:105-111 (1984)). For example, a polypeptide corresponding to a fragment of a polypeptide can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4diaminobutyric acid, alpha-amino isobutyric acid, 4aminobutyric acid, Abu, 2-amino butyric acid, g-Abu, e-Ahx, 6amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, alanine, fluoro-amino acids, designer amino acids such as methyl amino acids, C-methyl amino acids, N-methyl amino acids, and amino acid analogs in general. Furthermore, the amino acid can be D (dextrorotary) or L (levorotary).

### Methods of Use

In an aspect, the present application provides methods of contacting fibrin with an anti-fibrin antibody, such as a human or humanized antibody, which results in inhibition of microglial adhesion to the fibrin or fibrinogen γC domain.

In an aspect, the present application provides methods of using the isolated anti-fibrin antibodies described herein for treatment of a degenerative disorder of the nervous system. In certain aspects, described herein is a method for treating a degenerative disorder of the nervous system, the method comprising administering to a mammalian subject a therapeutically effective amount of an anti-fibrin antibody or pharmaceutical composition comprising an anti-fibrin antibody described herein. In certain embodiments, the present application provides methods of treating a degenerative disorder of the nervous system selected from the group consisting of: multiple sclerosis, spinal cord injury, stroke, and Alzheimer's Disease.

In certain aspects, described herein are methods for treating a pathology associated with Mac-1 binding to fibrin or Mac-1 binding with fibrinogen, the method comprising administering to a mammalian subject a therapeutically effective amount an isolated anti-fibrin antibody or a pharmaceutical composition comprising an isolated anti-fibrin antibody described herein.

In certain aspects, described herein are methods of inhibiting microglia activation, the method comprising administering to a mammalian subject a therapeutically effective amount an isolated anti-fibrin antibody or a pharmaceutical composition comprising an isolated antibody described herein.

In certain aspects, described herein is a method of preventing a degenerative disorder of the nervous system, the method comprising administering to a mammalian subject a therapeutically effective amount an isolated anti-fibrin antibody or a pharmaceutical composition comprising an isolated anti-fibrin antibody described herein. In certain embodiments, the present application provides methods of preventing a degenerative disorder of the nervous system selected from the group consisting of: multiple sclerosis, spinal cord injury, stroke, and Alzheimer's Disease.

In certain aspects, described herein are methods of treating or preventing colitis.

### Methods of Administration

In some embodiments, the methods provided herein are useful for the treatment of a degenerative nervous system disorder in an individual. In an embodiment, the individual is a human and the antibody is a fibrin antibody described herein.

In some embodiments, an antibody is administered intravenously, intramuscularly, subcutaneously, topically, orally, transdermally, intraperitoneally, intraorbitally, by implantation, by inhalation, intrathecally, intraventricularly, or intranasally. An effective amount of an anti-fibrin antibody may be administered for the treatment of cancer. The appropriate dosage of the anti-fibrin antibody may be determined based on the type of cancer to be treated, the type of the anti-fibrin antibody, the severity and course of the cancer, the clinical condition of the individual, the individual's clinical history and response to the treatment, and the discretion of the attending physician.

In some embodiments, an antibody provided herein is administered with at least one additional therapeutic agent. Any suitable additional therapeutic or immunotherapeutic agent may be administered with an antibody provided herein. Additional therapeutic agents include agents that are used to treat or prevent a degenerative disorder of the nervous system selected from the group consisting of: multiple sclerosis, spinal cord injury, stroke, and Alzheimer's Disease.

The additional therapeutic agent can be administered by any suitable means. In some embodiments, an antibody provided herein and the additional therapeutic agent are included in the same pharmaceutical composition. In some embodiments, an antibody provided herein and the additional therapeutic agent are included in different pharmaceutical compositions.

In embodiments where an antibody provided herein and the additional therapeutic agent are included in different pharmaceutical compositions, administration of the antibody can occur prior to, simultaneously, and/or following, administration of the additional therapeutic agent. In some embodiments, administration of an antibody provided herein and the additional therapeutic agent occur within about one month of each other. In some embodiments, administration of an antibody provided herein and the additional therapeutic agent occur within about one week of each other. In some embodiments, administration of an antibody provided herein and the additional therapeutic agent occur within about one day of each other. In some embodiments, administration of an antibody provided herein and the additional therapeutic agent occur within about twelve hours of each other. In some embodiments, administration of an antibody provided herein and the additional therapeutic agent occur within about one hour of each other.

### Kits and Articles of Manufacture

The present application provides kits comprising any one or more of the antibody compositions described herein. In some embodiments, the kits further contain a component selected from any of secondary antibodies, reagents for immunohistochemistry analysis, pharmaceutically acceptable excipient and instruction manual and any combination thereof. In one specific embodiment, the kit comprises a pharmaceutical composition comprising any one or more of the antibody compositions described herein, with one or more pharmaceutically acceptable excipients.

The present application also provides articles of manufacture comprising any one of the antibody compositions or kits described herein. Examples of an article of manufacture include vials (including sealed vials).

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1: Humanization of Anti-Fibrin Antibodies

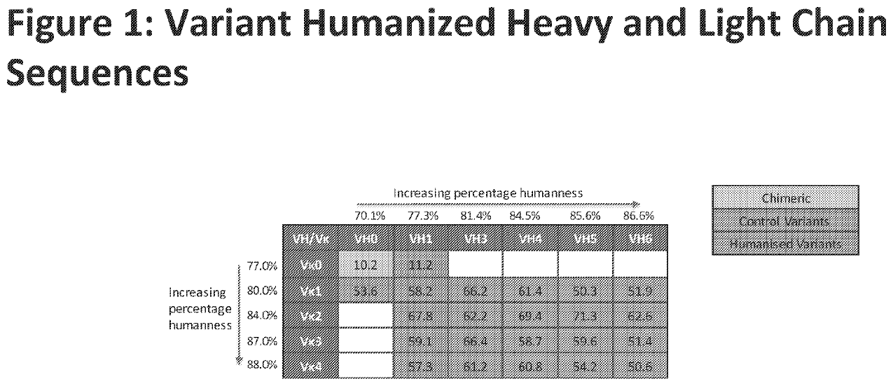

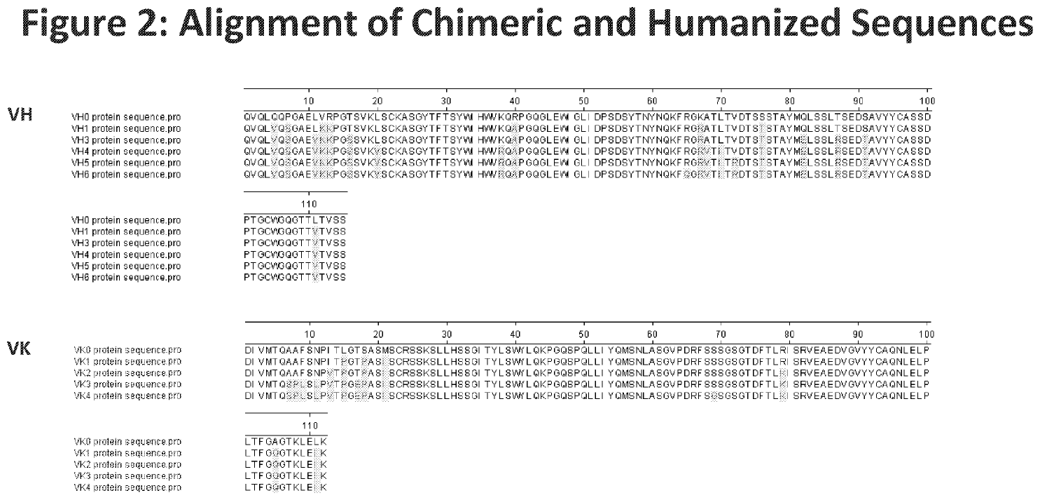

Composite Human Antibody™ VH and VL sequences were designed based on the mouse monoclonal anti-fibrin antibody (5B8) sequence (FIGS. 1-2). Structural models of the murine 5B8 antibody V regions were produced using Swiss PDB and analyzed in order to identify important “constraining” amino acids in the V regions that were likely to be essential for the binding properties of the antibody. Most residues contained within the CDRs (using both Kabat and Chothia definitions) together with a number of framework residues were considered to be important. The VH and VK sequences of murine 5B8 contain typical framework residues and the CDR 1, 2 and 3 motifs are comparable to many murine antibodies.

From the above analysis, it was considered that Composite Human sequences of 5B8 could be created with a wide latitude for alternative residues outside of the CDRs but with only a narrow menu of possible residues within the CDR sequences. Preliminary analysis indicated that corresponding sequence segments from several human antibodies could be combined to create CDRs similar or identical to those in the murine sequences. For regions outside of, and flanking the CDRs, a wide selection of human sequence segments were identified as possible components of the novel humanized V regions. It was noted during the analysis that there is a potential unpaired cysteine residue located within VH CDR3 (Kabat residue 102). Removing this residue is not possible during humanization; however, the removal of the cysteine should be considered as unpaired cysteines can lead to potential liabilities such as increased risk of aggregation.

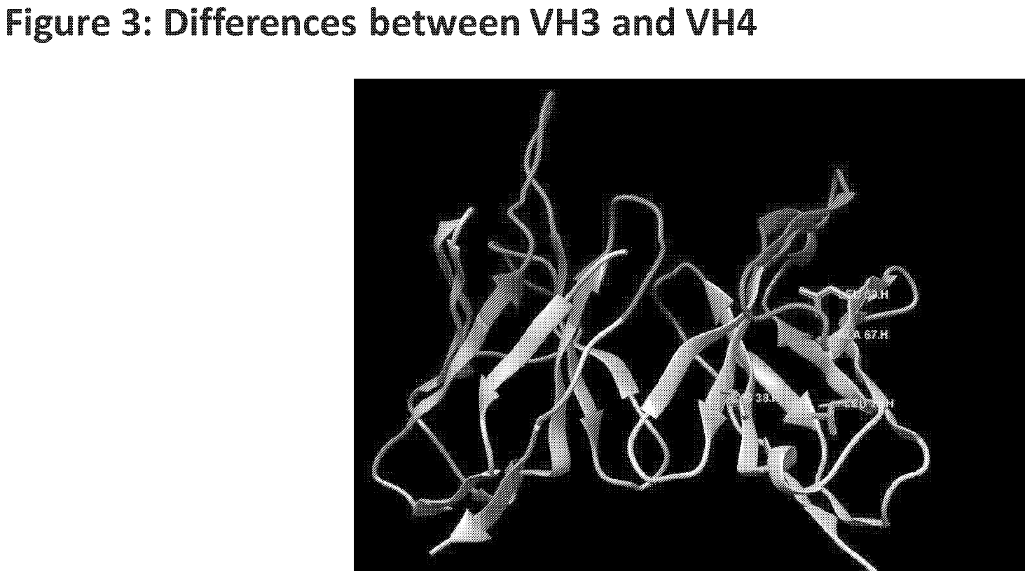

Based upon the structural analysis, a large preliminary set of sequence segments were identified that could be used to create 5B8 humanized variants. These segments were selected and analyzed using iTope™ technology for in silico analysis of peptide binding to human MHC class II alleles and using the TCED™ of known antibody sequence-related T cell epitopes. Sequence segments that were identified as significant non-human germline binders to human MHC class II or that scored significant hits against the TCED™ were discarded. This resulted in a reduced set of segments, and combinations of these were again analysed, as above, to ensure that the junctions between segments did not contain potential T cell epitopes. Selected sequence segments were assembled into complete V region sequences that were reduced in significant T cell epitopes. Initially, five heavy chain (VH1 to VH6; no VH2) and four light chain (Vκ1 to Vκ4) sequences were chosen for gene synthesis and expression in mammalian cells. A total of 23 variant combinations were expressed as IgG1 antibodies. FIG. 2 shows an alignment of the variable region sequences for the heavy and light chains for the chimeric antibody (V_{H}0 and Vk0) and humanized variants. FIG. 3 is a diagram illustrating the amino acid residue differences between VH3 and VH4.

### Example 2: Production and Characterization of Anti-Fibrin Antibodies

The number of variant combinations were narrowed down to a panel of six humanized variant antibodies that were purified. Transient transfections of antibody constructs were performed in HEK293 EBNA cells, and expression levels were assessed by Octet. Six lead humanized variants (VH3/Vκ1, VH3/Vκ2, VH4/Vκ1, VH4/Vκ2, VH5/Vκ2 and VH6/Vκ2) were transiently transfected into HEK EBNA adherent cells (LGC Standards, Teddington, UK) using a PEI transfection method in triple flasks and incubated for 10 days post-transfection. Due to the low expression of the 5B8 chimeric (V_{H}0/Vκ0) two batches were expressed. IgG supernatant titres were assessed on Day 10 by IgG titre ELISA.

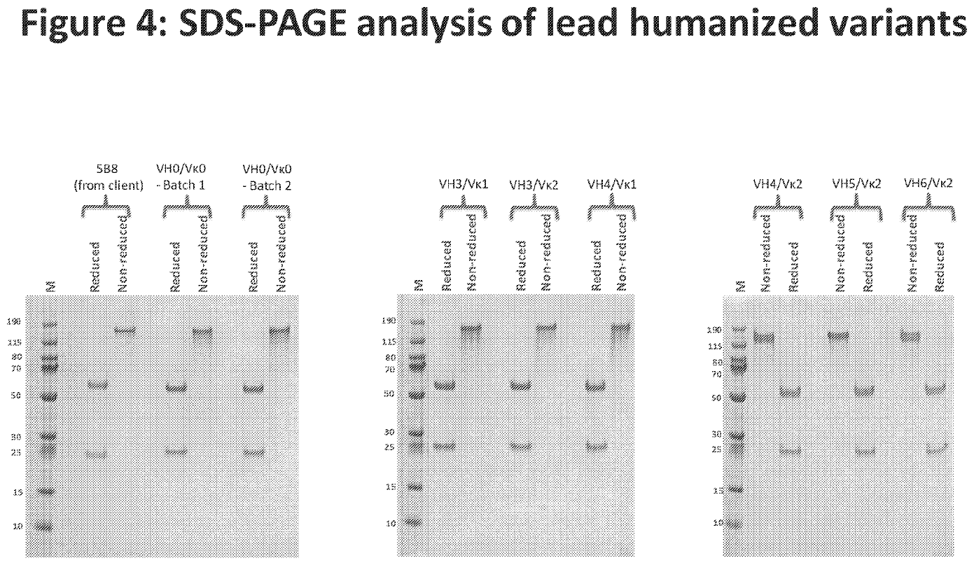

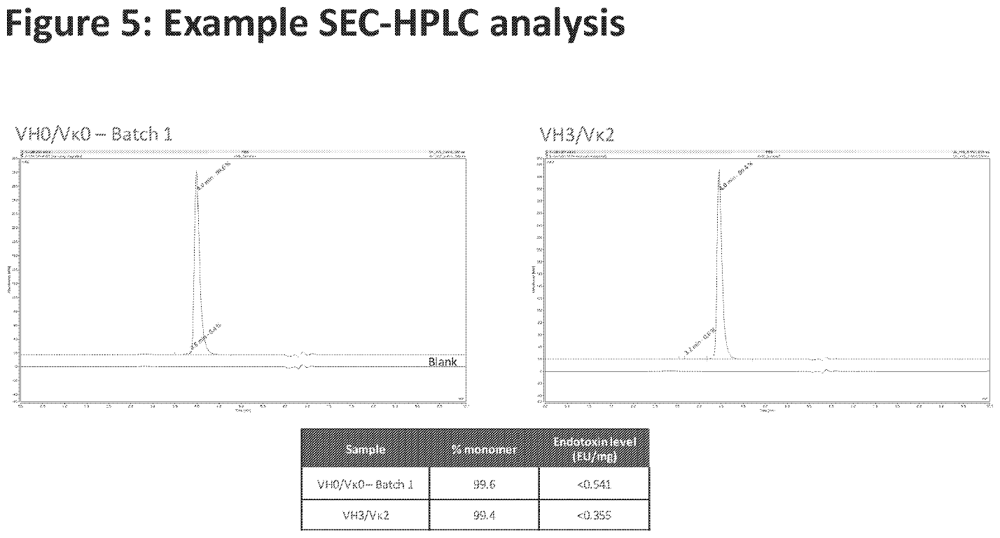

Antibodies were purified and SDS-PAGE analysis was performed on purified protein, verifying concentrations of antibodies for affinity analysis (FIG. 4). The 5B8 chimeric and lead humanized variant antibodies were purified from cell culture supernatant using 1 mL and 5 mL Mab Select Sure columns (GE Healthcare, Little Chalfont, UK) respectively. The column was washed using 1×TBS and protein eluted using Gentle elution Ag/Ab Elution buffer pH 6.6 (ThermoFisher, Loughborough, UK). Collected fractions were then further purified using a HiLoad™ 26/60 Superdex™ 200 pg preparative SEC column (GE Healthcare, Little Chalfont, UK) using PBS pH 7.4 as the mobile phase. Peak fractions containing monomeric protein were pooled, concentrated and filter sterilized before quantification by A280 nm using an extinction coefficient (Ec (0.1%)) based on the predicted amino acid sequence. Two μg of each purified protein was loaded per lane on a reducing and non-reducing SDS-PAGE. All samples were equally loaded showing that the concentrations of quantified antibodies were correct for affinity analysis. SEC-HPLC analysis was performed on select antibody variants (FIG. 5). Approximately 10 ug of antibody was analyzed by SEC-HPLC (post preparative SEC), indicating over 99% monomer purity in the purified protein samples and less than 0.6% endotoxin (EU/mg).

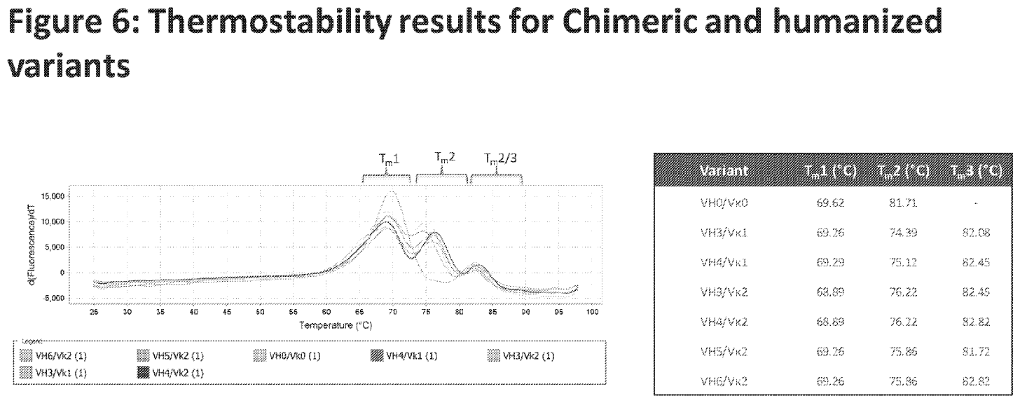

The six humanized variants were assessed for thermostability (FIG. 6). In order to assess the thermostability of the 5B8 chimeric and the six lead Composite Human Antibody™ variants; melting temperatures (the temperature at which 50% of a protein domain is unfolded) were determined using a fluorescence-based thermal shift assay. The six lead humanized antibodies, together with the irrelevant IgG1 standard (positive control), 5B8 chimeric (VH0/Vκ0) antibody and the murine 5B8 antibody, were diluted to a final concentration of 0.1 mg/ml in 1×DPBS containing SYPRO® Orange (ThermoFisher, Loughborough, UK) at a 1 in 1000 dilution and subjected to a temperature gradient from 25° C. to 99° C. on a StepOnePlus™ real-time PCR system (ThermoFisher, Loughborough, UK) over a period of 56 minutes. 1×DPBS was used as a negative control. The melting curves were analyzed using protein thermostability software (version 1.2) (FIG. 6). The negative control (PBS only) showed a constant low fluorescence, therefore the derivative data is close to zero and not shown in the melting curves in FIG. 6. The murine 5B8 antibody showed a lower first thermal transition (Tₘ1) than 5B8 chimeric and irrelevant IgG1 (FIG. 6). Additionally, the murine 5B8 displays two thermal transition peaks, however, the second unfolding event was not discrete enough for the software to assign a value. In contrast the chimeric showed two clearly distinguishable thermal transitions occurring at 69.6° C. (CH2 and Fab) and 81.7° C. (CH3). The overall Tm values suggests that the 5B8 chimeric is more stable than the murine 5B8 and the irrelevant IgG1 standard.

Typically, thermal denaturation of antibodies consists of three apparent transitions: one reversible transition of the CH2 domain and two consecutive irreversible transitions, reflecting the denaturation of the CH3 and Fab domains, respectively. The order of these transitions is usually CH2<Fab<CH3. The chimeric antibody shows two transitions while the humanised variants show three transitions and it is suggested that for the chimeric antibody, the CH2 and Fab domains have similar transition temperatures and hence overlap. Given the increase of the Tm2 (most likely correlating to the Fab domain) in the humanized variants

The results show that the first thermal transition (Tm1) occurs at a similar temperature for the chimeric and the lead humanized variants. The results show the following order of thermostability for the six select humanized variants: (Least) VH0/Vκ0<VH3/Vκ1<VH4/Vκ1<VH3/Vκ2<VH4/Vκ2<VH5/Vκ2<VH6/Vκ2 (most).

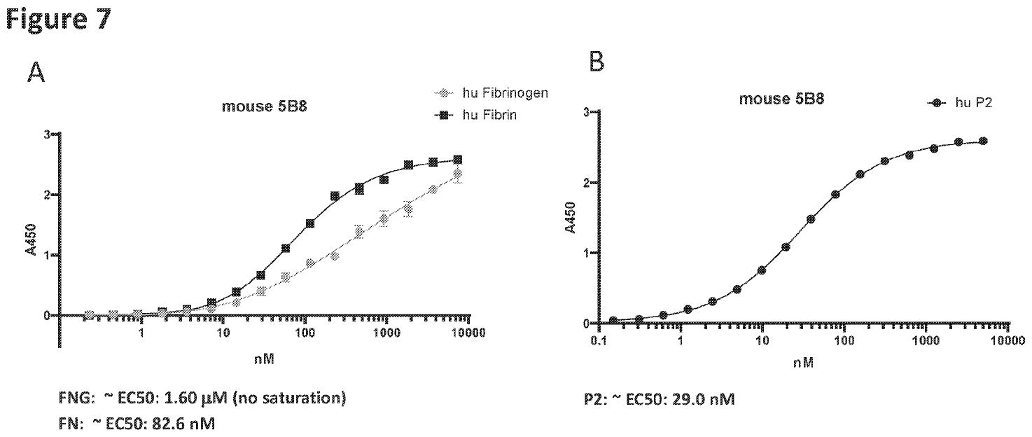

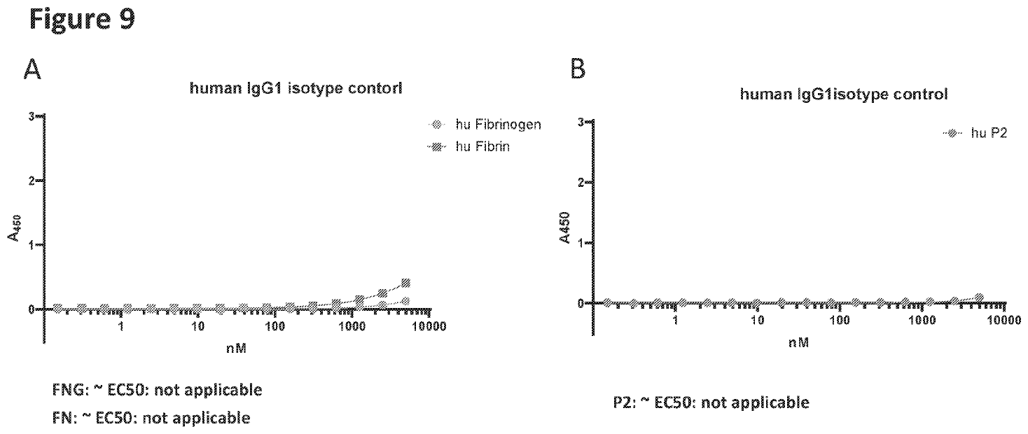

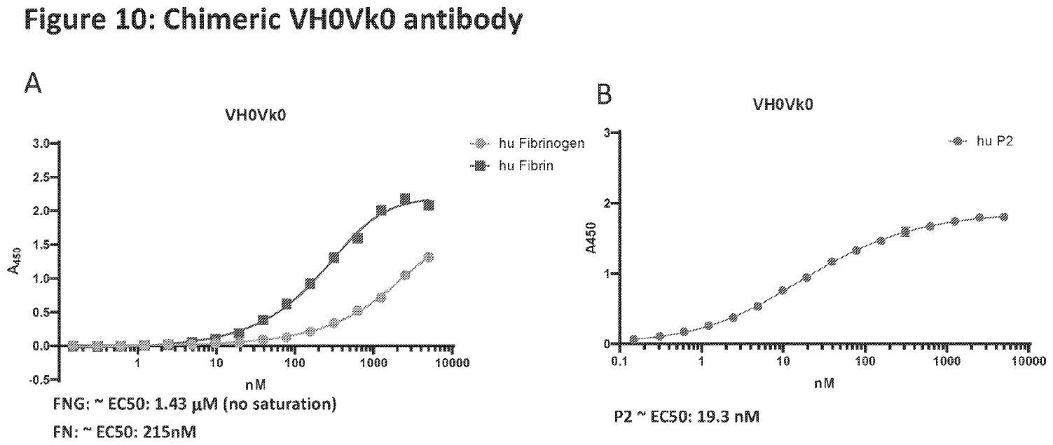

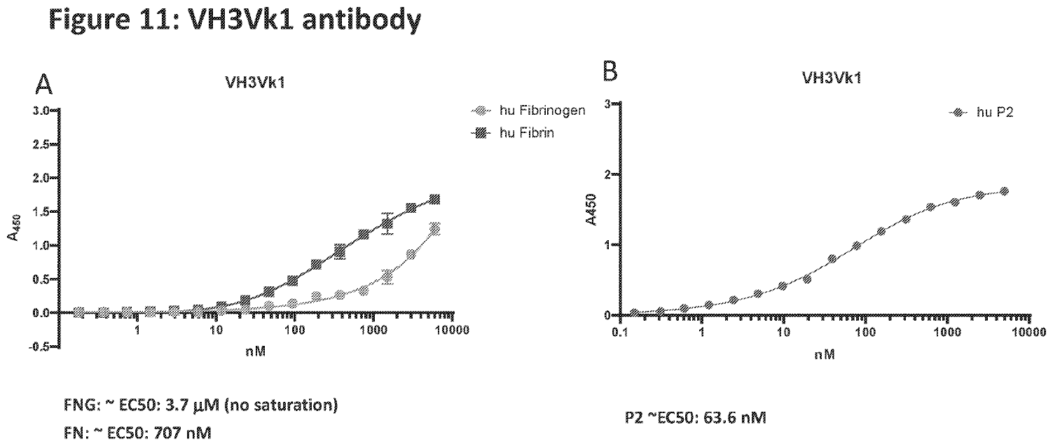

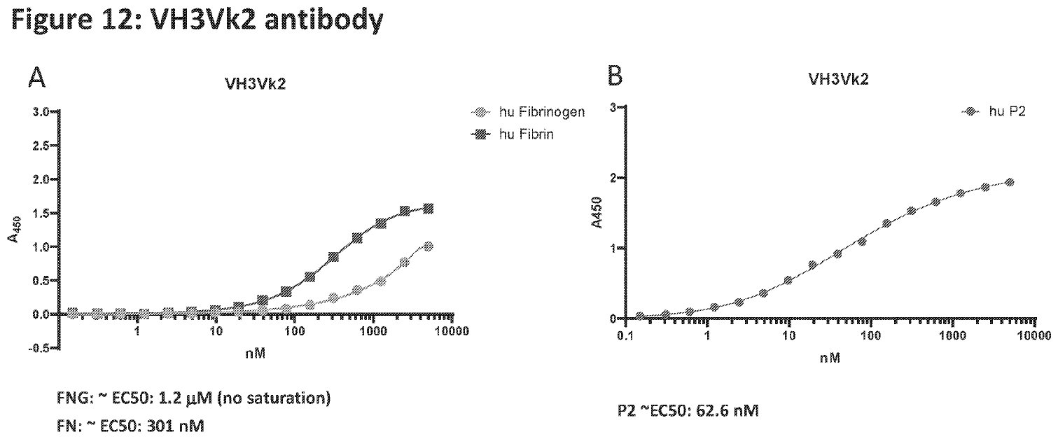

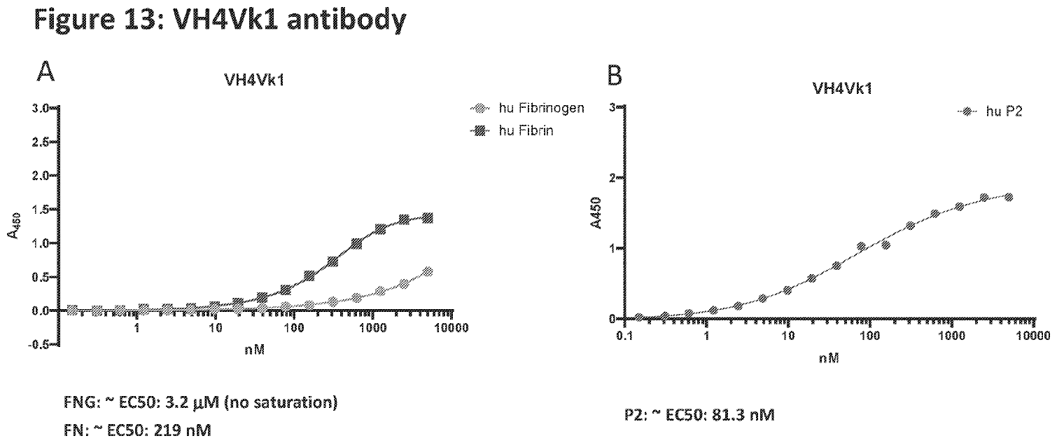

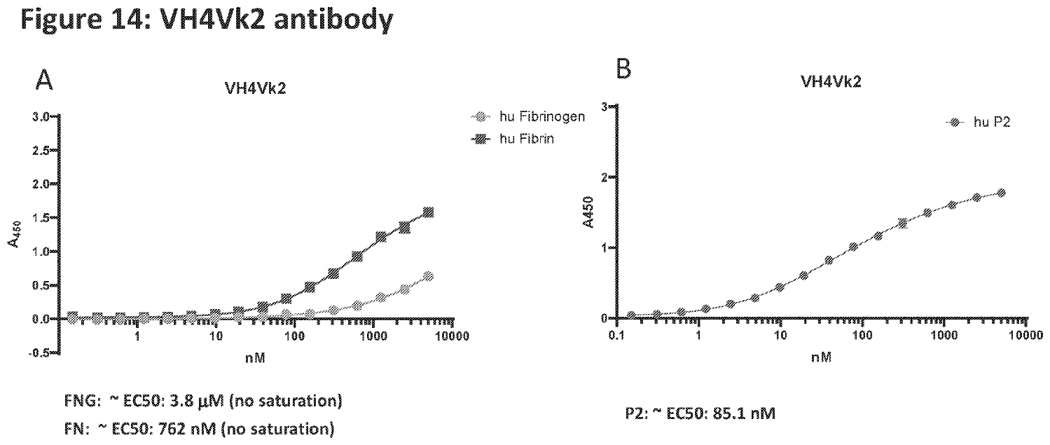

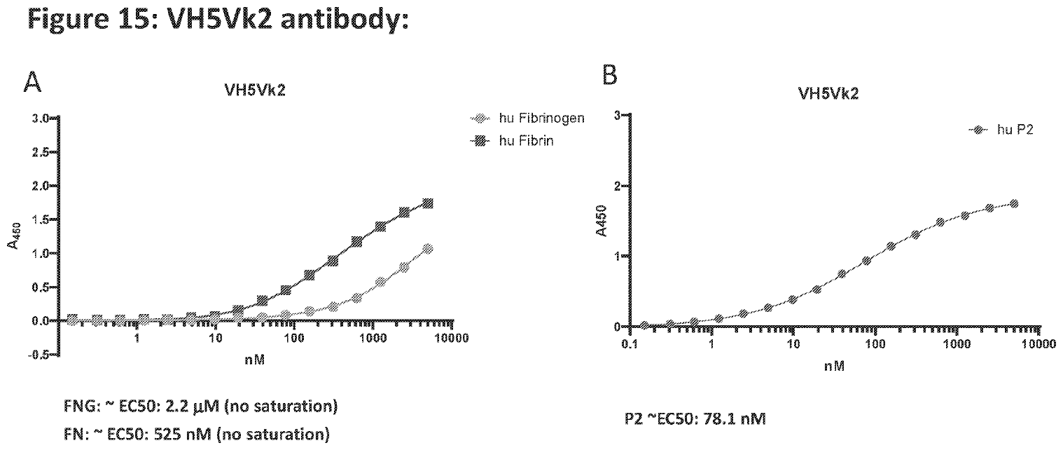

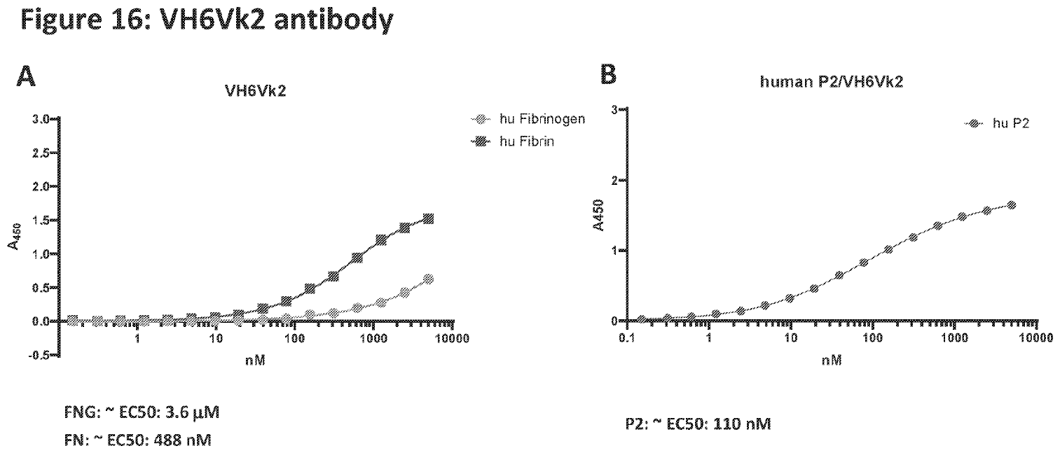

The humanized antibodies affinity for fibrinogen, fibrin and P2 peptide were assessed by Enzyme-linked immunosorbent assays (ELISA)s (FIGS. 7-16). ELISAs for fibrinogen binding were performed using plasminogen-depleted human Fibrinogen (EMD/Calbiochem). 100 mL of a 10 mg/mL IgG1-depleted Fibrinogen (FGN) in 20 mM HEPES/Saline buffer was added to each well of half of a 96-well Nunc Maxisorp™ ELISA plate. The ELISAs for fibrin binding were prepared by adding 50 mL of 20 mM HEPES/Saline buffer containing 14 mM CaCl₂, and 2 units thrombin to each well of the second half of the Nunc Maxisorp™ ELISA plate. 50 mL of 20 mg fibrinogen in 20 mM HEPES/Saline buffer were then added to a total volume of 100 mL. Plates were placed in a 37° C. incubator for 1 hour to allow fibrinogen to clot into fibrin and subsequently placed overnight into a plate dryer at 37° C. overnight. The ELISAs for P2 peptide were prepared by addition of 100 mL of 30 mM P2 peptide in 20 mM HEPES/Saline buffer to each well of a 96-well Nunc Maxisorp™ ELISA plate. Plates were transferred to the plate dryer and dried overnight at 37° C. All ELISAs were preformed using Nunc MaxiSorp 96-well ELISA plates. Blocking was performed using 100 mL 5% BSA in DPBS are incubated at RT for 1 hour. Plates were washed 5× in DPBS containing 0.05% Tween-20 (EL406 was used for the addition of wash buffer only). 50 mL of primary antibodies was diluted in 0.5% BSA in DPBS, and the primary antibodies were added to the plates and incubated for 2 hours at 37° C. Plates were washed 5× in DPBS/Tween. 100 mL secondary antibodies were diluted in 0.5% BSA in DPBS. The secondary antibodies were then added to the plates and incubated for 1 hour at RT. Afterwards, plates were washed 5× in DPBS/Tween. 100 mL of TMB/E substrate solution was added, and the reaction was stopped after 5 minutes with 1N HCl. The results of the ELISA assays (Table D) indicate that all of the humanized clones tested bind to P2 peptide and fibrin with similar increased affinity compared to their affinity to fibrinogen.

TABLE D
Approximate EC50 of Antibody Binding to
Human Fibrinogen, Fibrin and P2 peptide.

| | | | |
|---|---|---|---|
| Antibody | *Fibrinogen | Fibrin | P2 Peptide |

| | | | | | | |
|---|---|---|---|---|---|---|
| 5B8 | 1.6 | μM | 82.6 | nM | 29.0 | nM |
| VH0Vk0 | 1.4 | μM | 215 | nM | 19.3 | nM |
| VH3Vk1 | 3.7 | μM | 707 | nM | 63.6 | nM |
| VH3Vk2 | 1.2 | μM | 301 | nM | 62.6 | nM |
| VH4Vk1 | 3.2 | μM | 219 | nM | 81.3 | nM |
| VH4Vk2 | 3.8 | μM | 762 | nM | 85.1 | nM |
| VH5Vk2 | 2.2 | μM | 525 | nM | 78.1 | nM |
| VH6Vk2 | 3.6 | μM | 488 | nM | 110 | nM |

*Curves did not reach saturation

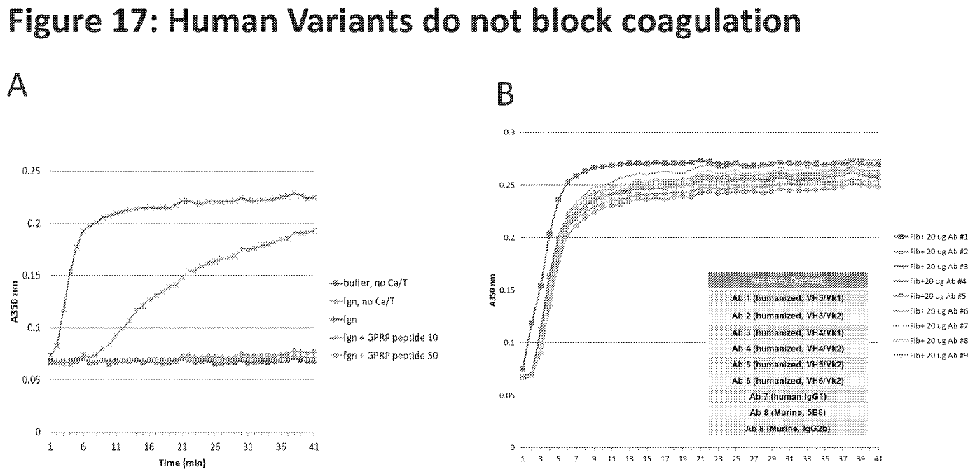

Coagulation assays (in vitro fibrin polymerization assays) were also performed with the humanized variants (FIG. 17). 20 mL of HEPES buffer was warmed in a 50 mL falcon tube in a laboratory bath with metallic beads (or water bath). This buffer was kept at 37° C. throughout the fibrin polymerization assay. Frozen fibrinogen vials were thawed in a laboratory bath. The fibrinogen was diluted in HEPES buffer to 0.656 mg/mL ( 1/38 dilution; 100 μL of fibrinogen stock solution was mixed with 3.7 mL of HEPES buffer). The fibrinogen was then mixed with the humanized variant antibodies, with or without pre-incubation for 3 h at 37° C. (a total volume of 160 μl). The frozen thrombin was thawed in a laboratory bath, and the thrombin was diluted in HEPES buffer (8 μL of thrombin stock solution was mixed with 152 μL of HEPES buffer). A mixture of CaCl₂) and thrombin in HEPES buffer was then prepared (a total of 3.6 mL: 72 mL of CaCl₂)+72 μL of thrombin dilution and 3456 μL of HEPES buffer). 40 μl of CaCl₂) and thrombin mixture was added it to each well. 160 μL of fibrinogen solution mixed with antibodies was added to each well of a 96-well plate containing the CaCl₂) and thrombin. This resulted in a final concentration in each well of: 10 mM CaCl₂), 0.3 U/mL thrombin, 150 μg/mL fibrinogen, 50 μg/mL humanized antibody. The final buffer concentration was: 20 mM HEPES, 150 mM NaCl, 5 mM eACA, pH 7.4. Controls for clot formation (fibrinogen without additions), controls with a known inhibitor of fibrinolysis (fibrinogen+GPRP), and antibody controls (fibrinogen+IgG control) were included. Duplicate wells were analyzed for each condition. Clot formation controls were loaded first and last on each experiment to indicate acceptable “apparent” differences in lag-time due to time difference between loading wells and starting absorbance readings. Background absorbance of soluble fibrinogen in absence of Ca/thrombin was very low (similar to buffer control). Absorbance was measured using a SpectraMax M5 Microplate Reader at A350 nm every 30 s for 40 min at 37° C. Upon addition of the CaCl₂) and thrombin, fibrinogen gets converted to polymerized fibrin resulting in increased turbidity, measured by an increase in absorbance at 350 nm, and the formation of a gel-like structure in the well. Once maximum fibrin polymerization reached, A350 nm stays stable (plateau).

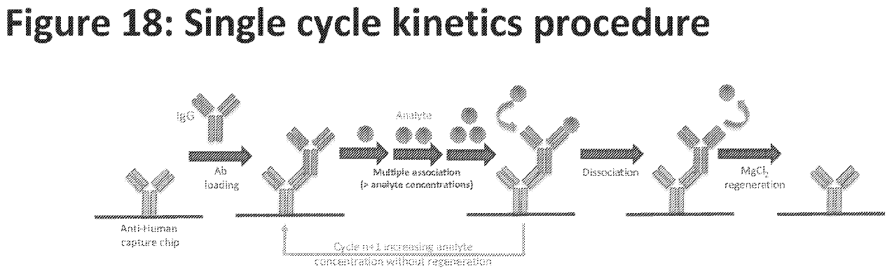

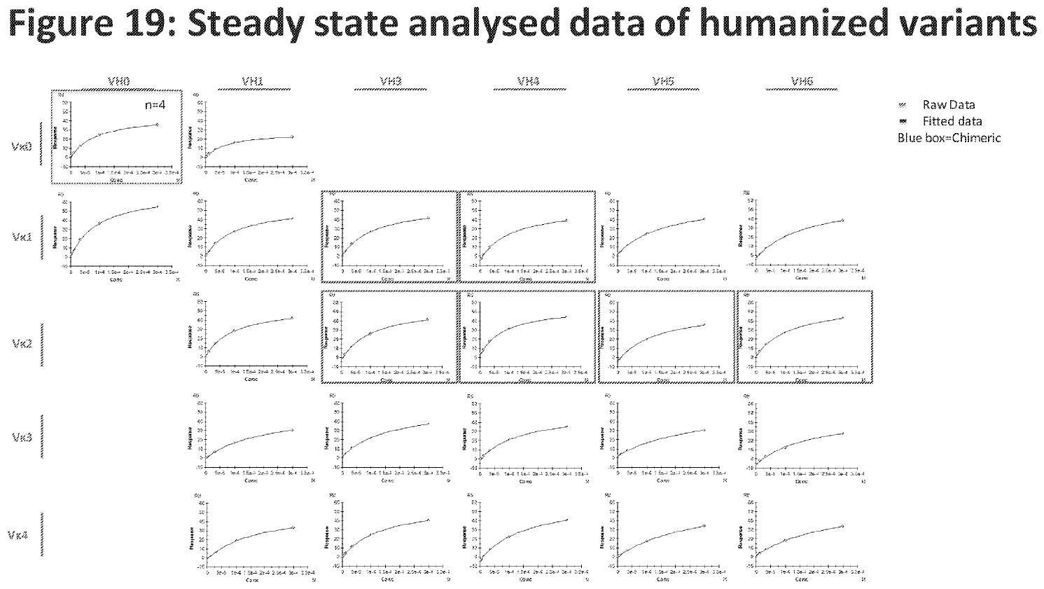

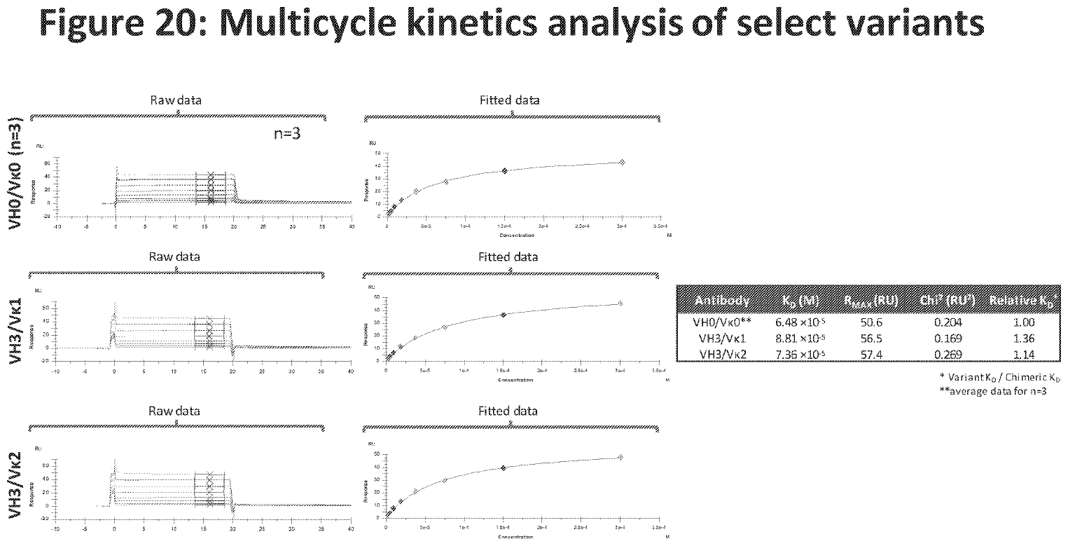

Antibody binding affinity analysis was conducted using HEK293 EBNA cell supernatants and affinity was assessed by Biacore single cycle kinetics (FIGS. 18 and 19) and multi-cycle kinetics analysis (FIG. 20). FIG. 18 illustrates the procedure used for the single cycle kinetics analysis. The Biacore T200 (U.S. Pat. No. 1,909,913) instrument was used for single cycle kinetics analysis with the control software v2.0.1, and evaluation software V3.0. The anti-human capture chip was used with a running buffer consisting of HBS-P+ buffer (pH 7.4) containing 0.1% BSA at 25° C. a flow rate of 30 μl/min. Neat HEK supernatants were used as ligand and loaded to ˜1674 RU at 10 μl/min. P2 peptide comprising SEQ ID NO: 31 (CPC Scientific 920712, Lot #CS-02-00350) was the analyte for the analysis. Five point 3-fold dilutions were run ranging from 300,000 nM to 3703 nM with an injection time of 20 s and a dissociation time of 25 s. Regeneration was performed with MgCl₂. Antibodies were diluted in running buffer to a final concentration of 20 μg/ml, based on concentrations assessed by Octet titre. At the start of each cycle, antibodies were loaded onto Fc2, Fc3 and Fc4 of the anti-human capture chip (GE Healthcare, Little Chalfont, UK). IgGs were captured at a flow rate of 10 μl/min to give an immobilization level (RL) of ˜ 1674 RU, the theoretical value to obtain an RMax of ˜50 RU. The surface was then allowed to stabilize. Single cycle kinetic data was obtained with P2 peptide as the analyte at a flow rate of 30 μl/min to minimize any potential mass transport limitations. Multiple repeats with the reference 5B8 chimeric antibody were performed to check the stability of the surface and analyte over the kinetic cycles. The signal from the reference channel Fc1 (no antibody) was subtracted from that of Fc2, Fc3 and Fc4 to correct for differences in non-specific binding to a reference surface. A five point, three-fold dilution range from 300,000 nM to 3703 nM of P2 peptide without regeneration between each concentration was used. The association phase for the five injections of increasing concentrations of P2 peptide was monitored for 20 seconds each time and a single dissociation phase was measured for 25 seconds following the last injection analyte. Regeneration of the anti-human capture surface was conducted using one injection of 3.8 M magnesium chloride. The signal from the reference channel Fc1 was subtracted from that of Fc2, Fc3 and Fc4 to correct for differences in non-specific binding to a reference surface. The data was then analyzed using a steady state fitting model due to the low affinity interaction.

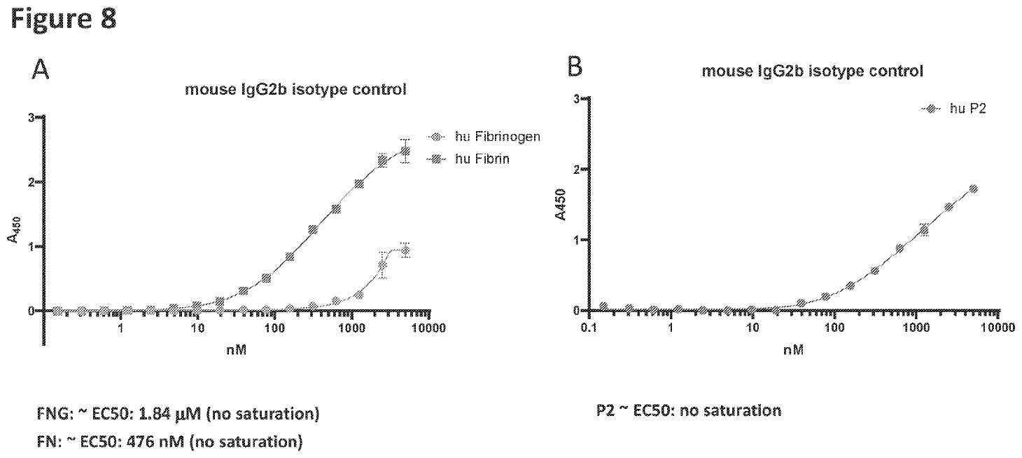

FIG. 8 shows the raw sensograms and fitted data for the single cycle kinetics binding to P2 peptide, and Table E summaries the kinetic parameters, expression levels and estimated K_{D} of the humanized variants obtained from the single cycle kinetics analyses. All variants containing Vκ1 and Vκ2 are within two-fold of the Chimeric (VH0/Vκ0). These results identified the six potential lead variants as: VH3/Vκ1, VH3/Vκ2, VH4/Vκ1, VH4/Vκ2, VH5/Vκ2 and VH6/Vκ2.

Multicycle kinetics analysis of the variants were also performed to determine antibody affinity. In order to establish an accurate affinity for P2 peptide, multicycle kinetics analysis was performed on the purified 5B8 Chimeric antibody and the six lead humanized variant antibodies using a Biacore T200 (U.S. Pat. No. 1,909,913) instrument running Biacore T200 Evaluation Software V3.0.1 (Uppsala, Sweden). At the start of each cycle, antibodies were loaded onto Fc2, Fc3 and Fc4 of the anti-human capture chip (GE Healthcare, Little Chalfont, UK). IgGs were captured at a flow rate of 10 μl/min to give an immobilization level (RL) of ˜1674 RU, the theoretical value to obtain a RMax of ˜50 RU. The surface was then allowed to stabilize. Kinetic data was obtained with P2 peptide as analyte using a flow rate of 30 l/min to minimize any potential mass transfer effects. Multiple repeats of a blank and a repeat of three concentrations of the analyte were programmed into the kinetic run in order to check the stability of both the surface and analyte over the kinetic cycles. For kinetic analysis, an eight point, two-fold dilution range was selected from 300,000 nM to 2343.75 nM of P2 peptide was used. The association phase of the peptide was monitored for 20 seconds and the dissociation phase was monitored for 25 seconds. Regeneration of the anti-human capture surface was conducted using one injection of 3.8 M magnesium chloride. The signal from the reference channel Fc1 was subtracted from that of Fc2, Fc3 and Fc4 to correct for differences in non-specific binding to a reference surface, and the data was then analyzed using steady state analysis due to the low affinity interaction to P2 peptide.

FIG. 20 shows the raw sensograms and fitted data from multi-cycle kinetics analyses of select variants for binding to P2 peptide, and Table F and Table G summarize the kinetics data from both single cycle and multi-cycle kinetics analyses. Table H summarizes the expression levels, binding affinity analysis and thermostability analysis for the six humanized variant antibodies. These results show that all of the humanized variants have a KD within ˜2-fold of the chimeric (VH0/Vκ0) antibody.

TABLE E
Steady state data summary of humanized variants

| | Expression | Estimated | | |
|---|---|---|---|---|
| | level (μg/mL) | K_{D} | Chi² | Relative |
| Antibody | Day 11 | (M) | (RU²) | K_{D} |
| VH0/VK0 | 10.20 | 8.87 × 10−⁵ | 46.10 | 1.00 |
| VH0/VK1 | 53.60 | 9.63 × 10−⁵ | 71.90 | 1.09 |
| VH1/VK0 | 11.20 | 7.65 × 10−⁵ | 28.00 | 0.86 |
| VH1/VK1 | 58.20 | 1.04 × 10⁻⁴ | 54.60 | 1.17 |
| VH1/VK2 | 67.80 | 9.64 × 10⁻⁵ | 54.40 | 1.09 |
| VH1/VK3 | 59.10 | 2.00 × 10−⁴ | 50.80 | 2.25 |
| VH1/VK4 | 57.30 | 1.76 × 10−⁴ | 55.10 | 1.98 |
| VH3/VK1 | 66.20 | 1.13 × 10−⁴ | 56.00 | 1.27 |
| VH3/VK2 | 62.20 | 1.05 × 10−⁴ | 59.20 | 1.18 |
| VH3/VK3 | 66.40 | 1.60 × 10⁻⁴ | 54.60 | 1.80 |
| VH3/VK4 | 61.20 | 1.35 × 10−⁴ | 58.90 | 1.52 |
| VH4/VK1 | 61.40 | 1.05 × 10−⁴ | 58.00 | 1.18 |
| VH4/VK2 | 69.40 | 8.45 × 10−⁵ | 53.60 | 0.80 |
| VH4/VK3 | 58.70 | 1.48 × 10⁻⁴ | 53.00 | 1.67 |
| VH4/VK4 | 60.80 | 1.57 × 10⁻⁴ | 67.70 | 1.77 |
| VH5/VK1 | 50.30 | 1.73 × 10−⁴ | 59.80 | 1.95 |
| VH5/VK2 | 71.30 | 1.30 × 10−⁴ | 56.90 | 1.47 |
| VH5/VK3 | 59.60 | 2.79 × 10−⁴ | 54.30 | 3.15 |
| VH5/VK4 | 54.20 | 2.77 × 10⁻⁴ | 64.70 | 3.12 |
| VH6/VK1 | 51.90 | 1.74 × 10⁻⁴ | 64.60 | 1.96 |
| VH6/VK2 | 62.60 | 1.24 × 10−⁴ | 57.90 | 1.40 |
| VH6/VK3 | 51.40 | 1.92 × 10−⁴ | 55.10 | 2.16 |
| VH6/VK4 | 50.60 | 2.65 × 10⁻⁴ | 60.70 | 2.99 |

TABLE F
Kinetics analysis of lead variant antibodies

| | Single cycle kinetics data | | Multicycle kinetics data | |
|---|---|---|---|---|
| Antibody | K_{D}(M) | Relative K_{D} | K_{D}(M) | Relative K_{D} |
| VH0/VK0 | 8.87 × 10⁻⁵ | 1.00 | 6.48 × 10⁻⁵ | 1.00 |
| VH3/VK1 | 1.13 × 10⁻⁴ | 1.27 | 8.81 × 10⁻⁵ | 1.36 |
| VH3/VK2 | 1.05 × 10⁻⁴ | 1.18 | 7.36 × 10⁻⁵ | 1.14 |
| VH4/VK1 | 1.05 × 10⁻⁴ | 1.18 | 8.62 × 10⁻⁵ | 1.33 |
| VH4/VK2 | 8.45 × 10⁻⁵ | 0.80 | 7.92 × 10⁻⁵ | 1.22 |
| VH5/VK2 | 1.30 × 10⁻⁴ | 1.47 | 1.22 × 10⁻⁴ | 1.88 |
| VH6/VK2 | 1.24 × 10⁻⁴ | 1.40 | 1.32 × 10⁻⁴ | 2.04 |

TABLE G
Summary of humanized lead data

| | iTope analysis (VH & Vk) | | 6 well | | | | | |
|---|---|---|---|---|---|---|---|---|
| | Promiscuous | Promiscuous | Expression | Multicycle kinetics data | | Thermostability data | | |
| | High | Moderate | levels | | Relative | Tₘ1 | Tₘ2 | Tₘ3 |
| Antibody | residues | residues | (μg/mL) | K_{D} (M) | K | (° C.) | (° C.) | (° C.) |
| VH0/VK0 | 17 | 7 | 10 | 6.48 × 10−⁵ | 1.00 | 69.62 | 81.71 | |
| VH3/Vk1 | 11 | 5 | 66 | 8.81 × 10−⁵ | 1.36 | 69.26 | 74.39 | 82.08 |
| VH3/Vk2 | 10 | 5 | 62 | 7.36 × 10⁻⁵ | 1.14 | 68.89 | 76.22 | 82.45 |
| VH4/Vk1 | 9 | 5 | 61 | 8.62 × 10−⁵ | 1.33 | 69.29 | 75.12 | 82.45 |
| VH4/Vk2 | 8 | 5 | 69 | 7.92 × 10 − 5 | 1.22 | 68.89 | 76.22 | 82.82 |
| VH5/Vk2 | 8 | 5 | 71 | 1.22 × 10−⁴ | 1.88 | 69.26 | 75.86 | 81.72 |
| VH6/Vk2 | 7 | 4 | 62 | 1.32 × 10⁻⁴ | 2.04 | 69.26 | 75.86 | 82.82 |
| Murine 5B8 | | | | 9.15 × 10−⁵ | | 64.55 | | |
| (from client) | | | | | | | | |

TABLE H
Steady state data and expression summary

| | Expression level | | | | |
|---|---|---|---|---|---|
| Antibody | (μg/mL) Day 7* | K_{D}(M) | R_{MAX} (RU) | Chi² (RU²) | Relative K_{D} |
| VH4/VK2 | 16.4 | 4.7-05 | 133.3 | 4.5 | 1.00 |
| VH4 C102A/VK2 | 37.9 | 7.2E−05 | 127.2 | 2.4 | 1.53 |
| VH4 C102G/VK2 | 24.5 | 2.2E−05 | 128 | 7.0 | 0.59 |
| VH4 C102L/VK2 | 42.8 | 9.3E−05 | 134.9 | 2.2 | 1.98 |
| VH4 C102S/VK2 | 27.6 | 6.6E−05 | 128.9 | 4.6 | 1.41 |
| VH4 C102T/VK2 | 26.5 | 4.9E−05 | 145.8 | 5.5 | 1.05 |
| VH4 C102V/VK2 | 33.6 | 3.3E−05 | 100.2 | 12.9 | 0.70 |
| VH5/VK2 | 8.8 | 7.9E−05 | 139.9 | 3.0 | 1.00 |
| VH5 C102A/VK2 | 39.8 | 1.1E−04 | 142.2 | 0.03 | 1.44 |
| VH5 C102G/VK2 | 59.3 | 3.9E−05 | 159.4 | 2.9 | 0.50 |
| VH5 C102L/VK2 | 50.8 | 1.4E−04 | 167.3 | 0.2 | 1.77 |
| VH5 C102S/VK2 | 41.8 | 1.1E−04 | 157 | 1.0 | 1.33 |
| VH5 C102T/VK2 | 59.0 | 8.0E−05 | 177.2 | 1.6 | 1.01 |
| VH5 C102V/VK2 | 34.5 | 4.4E−05 | 115.3 | 4.5 | 0.56 |

### Example 3: Substitution of Cysteine 102 Improves Anti-Fibrin Binding Affinity to Fibrin γ377-395 Epitope

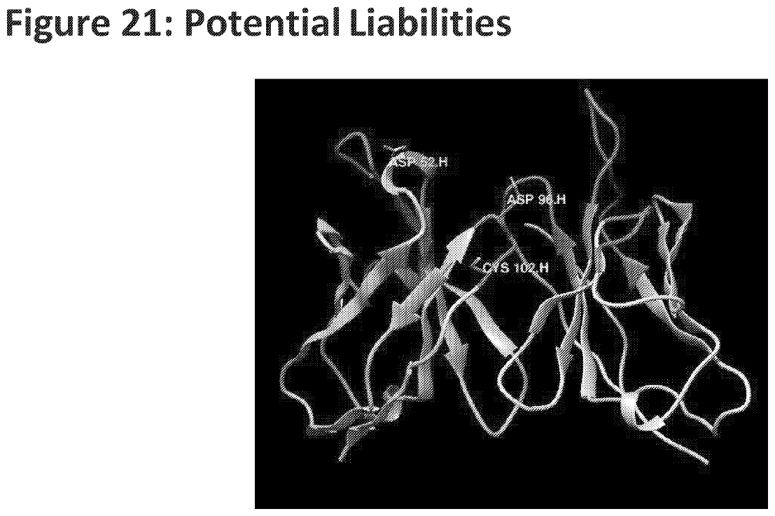

Three potential liabilities were identified within the V_{H} CDRs of the parental antibody sequence and that could not addressed by humanization (FIG. 21). Free Cysteine was identified at Cysteine 102 of CDR-H3 (SEQ ID NO: 3) and acid labile sites Asp 52 of CDR-H2 and Asp 96 of CDR-H3.

Cysteine 102 of the CDR-H3 (SEQ ID NO: 3) sequence liability was originally identified during humanization of 5B8 mouse monoclonal anti-fibrin antibody. To address the cysteine 102 liability in two lead humanized variants VH4/Vκ2 and VH5/Vκ2 (IgG1), site directed mutagenesis was performed. A total of 6 substitutions (C102A, C102G, C102L, C102S, C102T and C102V) were performed on either VH4 (SEQ ID NO: 7) or VH5 (SEQ ID NO: 14) to produce a total of 12 new VH sequences. Twelve new antibodies were produced using the 12 VH sequences and Vκ2 light chain sequence (SEQ ID NO: 21). These 12 new antibodies were transiently transfected into HEK EBNA cells using a PEI method at small scale (6 well) together with the parental humanized variants VH4/Vκ2 and VH5/Vκ2. Supernatants were harvested seven days post-transfection and Octet titers and binding of fibrin peptide γ377-395 (SEQ ID NO: 31) was assessed by Biacore single cycle kinetics using steady state affinity.

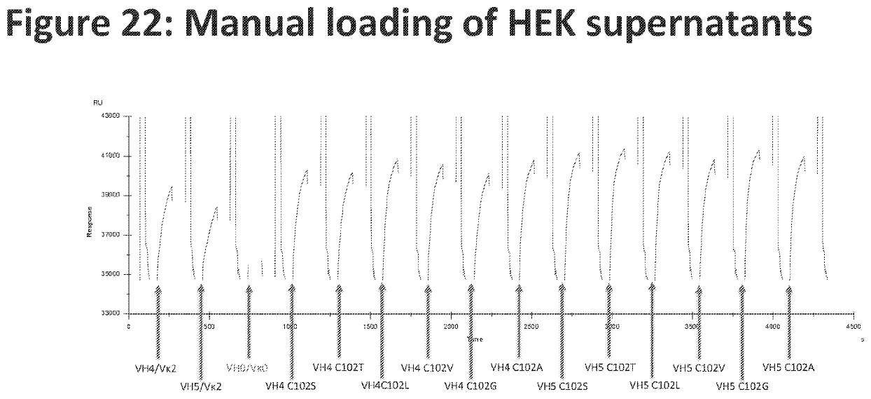

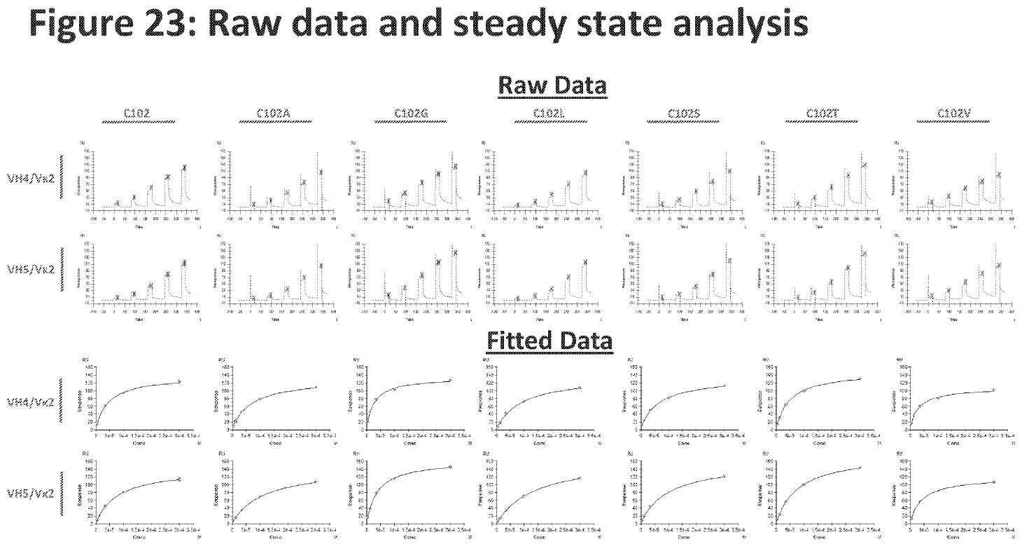

To calculate contact times for Biacore antibody loading, HEK supernatants were injected for 90 sec at 10 ml/min (FIG. 22). The signal following the injection is an indication of antibody expression levels. The chimeric antibody which was transfected at the same time as the other variants gave a very low signal. As a result, sufficient capture levels could not be achieved (low expression was confirmed by Octet which showed the chimeric was below the detection level). For Biacore analysis, antibody variants harboring cysteine substitutions were compared to the corresponding (unmodified) humanized variant (either V_{H}4/Vκ2 or V_{H}5/Vκ2). FIG. 23 shows the results of the steady state analysis using single cycle kinetics for 12 humanized variant antibodies with the cysteine substitutions, and Table G summarizes the expression levels and binding affinity for the twelve humanized variant antibodies with the cysteine substitutions. These results show that antibodies containing all six C102 substitutions were able to bind fibrin peptide γ377-395 (SEQ ID NO: 31) within ˜2-fold of the corresponding parental antibody. KD values were consistent with those observed previously using supernatants, 8.5×10⁻⁵ (VH4/Vκ2) and 1.3×10⁻⁴ (VH5/Vκ2). C102G was identified to be the best substitution for both VH4/Vκ2 and VH5/Vκ2 (C102G<C102V<C102T<C102S<C102A<C102L). Unexpectedly, humanization improved antibody expression, and this was further improved with removal of free cysteine at cysteine 102 of CDR-H3.

### Example 4: The Humanized Antibody Variants have Similar Activity to 5B8 in Fibrinogen-Induced Encephalomyelitis (FIE)

The effect of I.C. V. injection of humanized anti-fibrin antibody variant V_{H}5 C102G/Vκ2 on microglia activation, oxidative stress, and macrophage recruitment was assessed. Plasminogen-free fibrinogen was dissolved in endotoxin-free distilled water, diluted to 5 mg/ml with artificial cerebral spinal fluid (ACSF). Fibrinogen (1 μl of 5 mg/ml) was injected at a rate of 0.3 μl/min with a 10-μl Hamilton syringe attached to a 33 gauge needle into the brain at coordinates: anteroposterior, −1.0 mm; mediolateral, −0.7 mm; dorsoventral, −1.325 mm from the bregma, according to Paxinos and Watson. For prophylactic intracerebroventricular (i.e.v.) injections, 10 μg of antibodies were delivered at a rate of 0.3 l/min) with a 10-μl syringe attached to a 33 gauge needle into the cerebral ventricle (anteroposterior, −2.0 mm; mediolateral, 0 mm, dorsoventral, −2.0 mm) 30 min before fibrinogen injection. For prophylactic intravenous (i.v.) injections, antibodies were injected retro-orbitally with a 0.3 mL 29 g insulin syringe 1 hr before fibrinogen injection. Mice were sacrificed 3 days after fibrinogen injection for brain histopathological assessment.

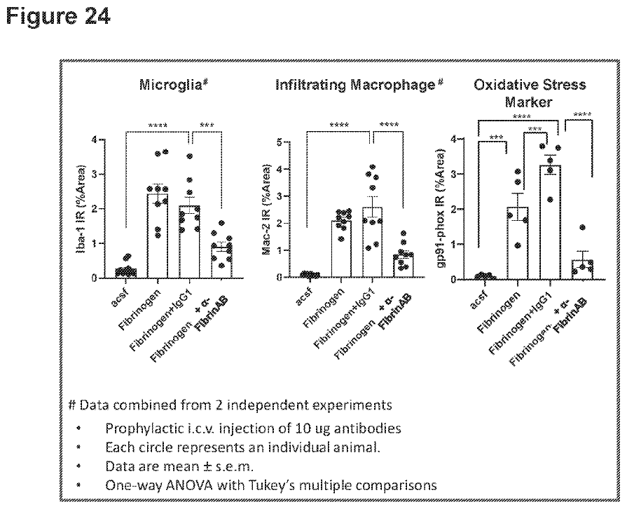

10 ug antibodies were administered prophylactically by i.e.v. injection to FIE mice (FIG. 24). Stereotaxic fibrinogen was injected into the corpus callosum to induce encephalomyelitis. Each circle represents an individual animal. Data are mean+s.e.m. One-way ANOVA with Tukey's multiple comparisons.

This data confirms a humanized antibody variant described herein inhibits microglial activation, macrophage recruitment and oxidative stress in FIE in vivo.

### Example 5: The Humanized Antibody Variant Co-Localizes with Fibrinogen in Experimental Autoimmune Encephalomyelitis (EAE)

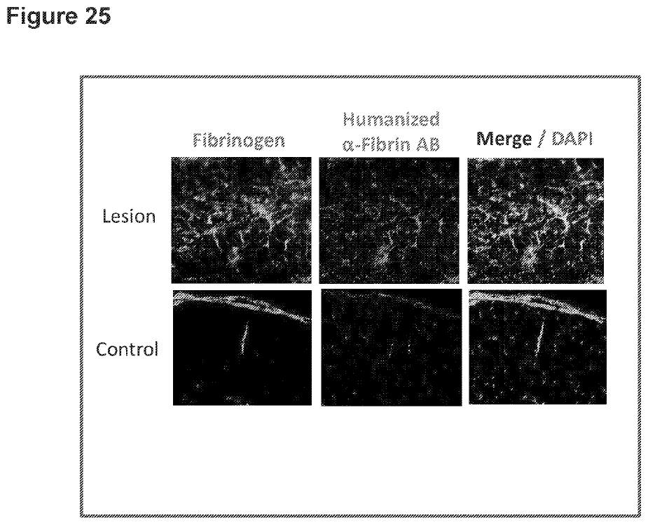

Tissue sections from mice with chronic EAE harboring fibrinogen accumulation in spinal cord lesions were stained with 10 mg/ml VH5 C102G/Vκ2-biotin and CY3-streptavidin antibody (FIG. 25).

Chronic EAE was induced by the epitope of amino acids 35-55 of myelin oligodendrocyte glycoprotein (MOG) (‘MOG35-55 EAE’) in 8-9 week old female SJL/J mice by subcutaneous immunization with 15 μg PLP139-151 in complete Freund's adjuvant supplemented with 400 μg of heat-inactivated Mycobacterium tuberculosis H37Ra (Day 0). Two days after immunization, mice were injected with 5 ng pertussis toxin via IP administration. Antibodies were administered at 0.2, 1, or 5 mg/kg IP prophylactically twice per week starting on day 0. Dexamethasone (0.5 mg/kg) was administered IP daily as a positive control. EAE disability scores were monitored daily up to the end of the study. The study was terminated 3 days post peak-EAE at around day 14-16 of the study and spinal cords were collected for histopathological analysis.

This data confirms that a humanized antibody variant described herein co-localizes with fibrin(ogen).

### Example 6: Pharmacokinetic Analysis of Humanized Antibody Variants

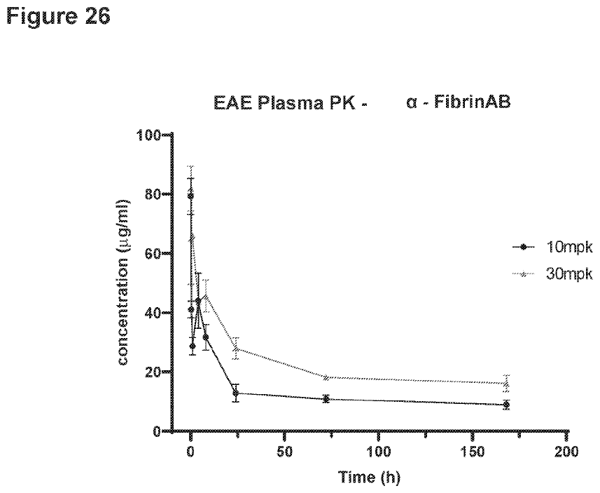

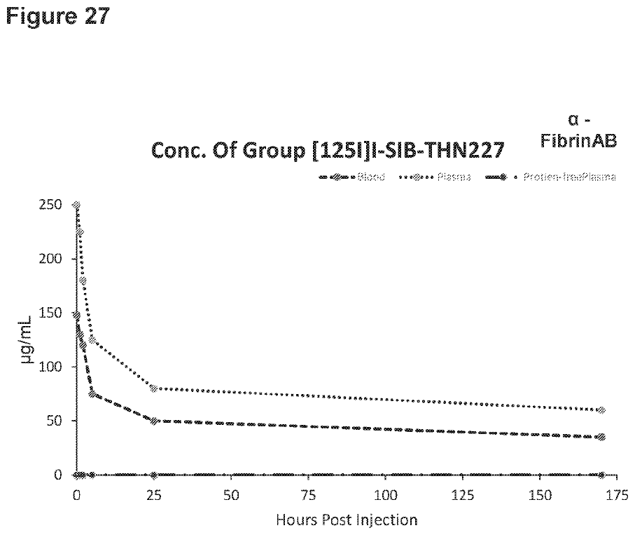

Pharmacokinetic analysis was performed using humanized anti-fibrin antibody variant VH5 C102G/Vκ2. Antibodies were detected via ELISA in plasma from EAE mice that have been administered either 10 mg/Kg or 30 mg/Kg VH5 C102G/Vκ2 antibody (FIG. 26). Antibodies were detected via ELISA in plasma and blood from wild-type Balb/c mice that have been administered VH5 C102G/Vκ2 antibody (FIG. 27). Similar pharmacokinetic trends were observed between BioAgilytix and Invicro, despite different baselines, disease models, and analytical methods.

These results show efficient metabolism and/or clearance of the humanized antibody variant at the dosages shown.

### Example 7: Therapeutic Treatment of Fibrinogen-Induced Encephalomyelitis (FIE)

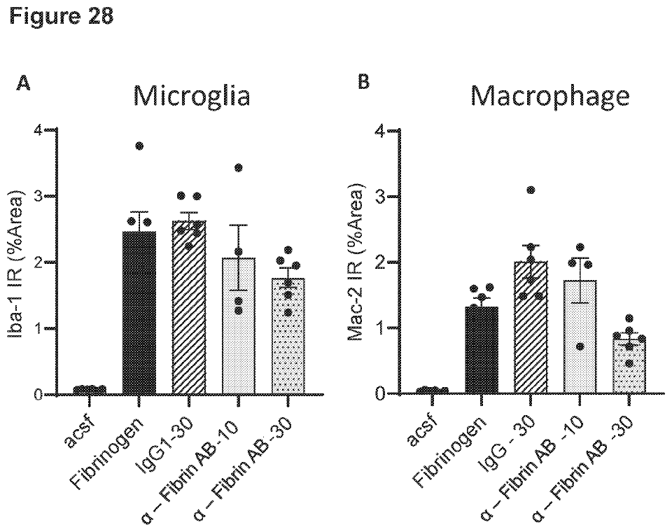

The ability of the humanized anti-fibrin antibodies to therapeutically inhibit microglia activation (FIG. 28A) and macrophage infiltration (FIG. 28B) in a fibrinogen-induced encephalomyelitis (FIE) mouse model was then assessed. To induced FIE, mice were anaesthetized with avertin and placed in a stereotactic apparatus. Plasminogen-free fibrinogen was dissolved in endotoxin-free distilled water, diluted to 5 mg/ml with artificial cerebral spinal fluid (ACSF). Fibrinogen (1 μl of 5 mg/ml) was injected at a rate of 0.3 μl/min with a 10-μl Hamilton syringe attached to a 33 gauge needle into the brain at coordinates: anteroposterior, −1.0 mm; mediolateral, −0.7 mm; dorsoventral, −1.325 mm from the bregma, according to Paxinos and Watson.

For prophylactic intracerebroventricular (i.e.v.) injections, 10 μg of antibodies were delivered at a rate of 0.3 μl/min) with a 10-μl syringe attached to a 33 gauge needle into the cerebral ventricle (anteroposterior, −2.0 mm; mediolateral, 0 mm, dorsoventral, −2.0 mm) 30 min before fibrinogen injection. For prophylactic intravenous (i.v.) injections, antibodies were injected retro-orbitally with a 0.3 mL 29 g insulin syringe 1 hr before fibrinogen injection.

Stereotaxic fibrinogen was injected into the corpus callosum to induce encephalomyelitis. A total of 78 mice, separated into 13 groups: n=6 mice per group were then injected i.v. with the V_{H}5 C102G/Vκ2 humanized antibody at either 10 mg/kg or 30 mg/kg. Brain tissue harvesting and preparation was performed three days post-injection. Sample exclusion: 5 mice; found dead at day 1 post-surgery (C 10 mg/kg, n=1) and day 2 post-surgery (B 10 mg/kg, n=1; D 10 mg/kg, n=1). Wrong site injection (B 10 mg/kg, n=1; D 10 mg/kg, n=1). Blinding & Quantification: all FIE experiments, image collection and quantification performed in a blinded manner. Immunohistochemistry (IHC) and quantification was performed as follows: 73 mice samples were included for IHC and quantification. Coronal sections (30 um) were prepared on the Cryostat. Tissues were stained with Iba-1 (microglia marker, at a dilution of 1:750) and Mac-2 (macrophage infiltration marker, at a dilution of 1:750). The immunoreactivity of Iba-1 (Iba-1+ area) and Mac-2 (Mac-2+ area) was then calculated. A decrease in both microglia and macrophages were detected in tissues from mice treated with humanized anti-fibrin antibody variant

These results show the humanized antibody variants described herein can therapeutically reduce microglia and macrophage infiltration of mice with FIE.

### Example 8: Prophylactic Treatment of Relapsing-Remitting EAE

The ability of the humanized anti-fibrin antibodies to prophylactically treat Relapsing-Remitting EAE induced by the epitope of amino acids 139-151 of proteolipid protein (PLP) (‘PLP139-151 EAE’) was assessed. EAE was induced in 8-9 week old female SJL/J mice by subcutaneous immunization with 15 μg PLP139-151 in complete Freund's adjuvant supplemented with 400 μg of heat-inactivated Mycobacterium tuberculosis H37Ra (Day 0). 2 days after immunization, mice are injected with 5 ng pertussis toxin via IP administration. Antibodies were administered at 0.2, 1, or 5 mg/kg IP prophylactically twice per week starting on day 0. Dexamethasone (0.5 mg/kg) was administered IP daily as a positive control. Experimental Design: 6 groups: n=10 mice per group, a total of 60 mice. Dose regimen: Dexamethasone (5 mg/kg, daily), humanized anti-fibrin antibodies (A, B, C, D_5 mg/kg, every 3 days). EAE disability scores were monitored daily up to the end of the study. The study was terminated 3 days post peak-EAE at around day 14-16 of the study and spinal cords were collected for histopathological analysis.

Sample exclusion: 3 mice; found dead at day 12 (antibody B, n=1), day 15 (antibody C, n=1), or day 16 (antibody A, n=1). Blinding & Qunatification: All EAE experiments (antibody treatment and clinical score) were performed in a blinded manner. 57 spinal cord samples were prepared for tissue processing.

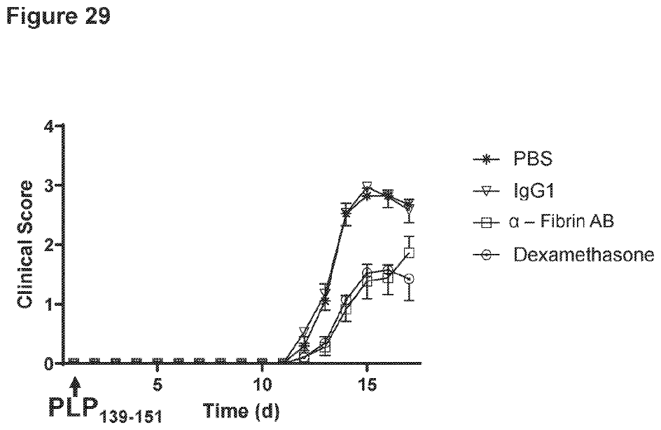

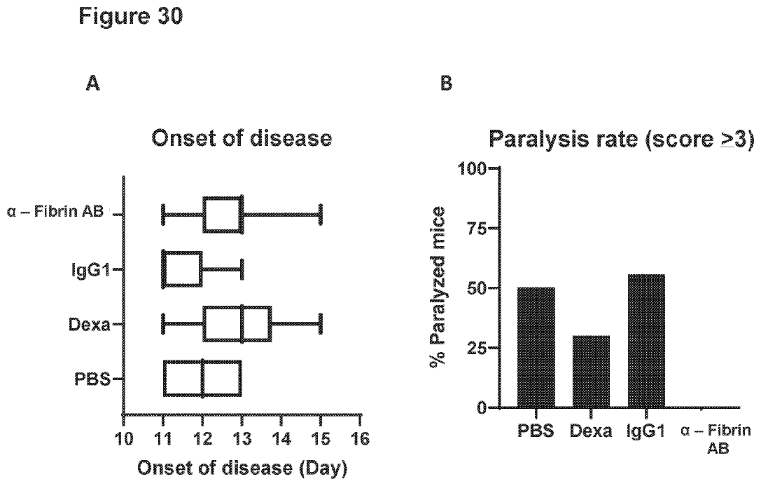

Clinical score of PLP EAE was assessed in mice that were prophylactically injected with antibodies (5 mg/kg i.p. every 3 days) (FIG. 29). The clinical score of mice that had been injected with anti-fibrin humanized antibody was reduced compared to control mice injected with PBS or IgG1 alone. Time to onset of disease was also assessed (FIG. 30A). The were no mice with paralysis that had been injected with anti-fibrin humanized antibody compared to control mice injected with PBS, IgG1, or dexamethasone alone which had between 25% and over 50% of mice with paralysis (FIG. 30B).

Taken together, these results show that the anti-fibrin humanized antibody is effective for prophylactic treatment of encephalomyelitis.

### Example 9: Treatment of Neurodegenerative Disaese

The purified humanized antibody variants described herein are formulated into a pharmaceutical composition to be administered to patients for the treatment of a neurodegenerative disease (e.g., multiple sclerosis or Alzheimer's Disease). The pharmaceutical composition comprising a humanized antibody variant described herein is administered at a dose sufficient to effectively reduce the symptoms of the neurodegenerative disease. The pharmaceutical composition is well tolerated and does not induce significant harmful adverse effects in the patient.

### Example 10: Humanized Antibody Variant for Treatment of Colitis

To initiate dextran sodium sulfate (DSS) induced colitis treatment, 8-10 week old female C57BL/6 mice are acclimated to the vivarium for at least 4 days, weighed, and randomized into treatment groups based on body weight. 2 types of studies were conducted: acute (7 days) and chronic (28 days).

The acute DSS study is performed by adding 2.5% DSS into drinking water for 7 days. Antibodies are administered IP every 2 days (Q2D) at 10 and 30 mg/kg. Mice are euthanized on day 7 with isoflurane anesthesia, exsanguination, followed by cervical dislocation. The colon was removed and analyzed for histopathology.

The chronic DSS study is performed by adding 2.0% DSS into drinking water for 1 week followed by replacing with 1 week of normal drinking water, followed by another week of 2% DSS and ending with another week of normal drinking water. Humanized antibody variants described herein are administered IV prophylactically starting on day 0 twice a week at 30 and 5 mg/kg. Mice are euthanized after 28 days with isoflurane anesthesia, exsanguination, followed by cervical dislocation. The colon is removed and analyzed for histopathology.

This study confirms that the humanized antibody variants described herein are effective for the treatment of colitis.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

| Description | Sequence | SEQ ID NO |
|---|---|---|
| CDR-H1 | GYTFTSYWIH | SEQ ID NO: 1 |
| CDR-H2 | LIDPSDSYTNYNQKFRG | SEQ ID NO: 2 |
| CDR-H3 | SDPTGX | SEQ ID NO: 3 |
| CDR-L1 | RSSKSLLHSSGITYLS | SEQ ID NO: 4 |
| CDR-L2 | QMSNLAS | SEQ ID NO: 5 |
| CDR-L3 | AQNLELPLT | SEQ ID NO: 6 |
| VH4-102C | QVQLVQSGAEVKKPGSSVKVSCKASGYTFTSYWIHWV | SEQ ID NO: 7 |
| | RQAPGQGLEWIGLIDPSDSYTNYNQKFRGRVTITVDTST | |
| | STAYMELSSLRSEDTAVYYCASSDPTGCWGQGTTVTVSS | |
| VH4-102A | QVQLVQSGAEVKKPGSSVKVSCKASGYTFTSYWIHWV | SEQ ID NO: 8 |
| | RQAPGQGLEWIGLIDPSDSYTNYNQKFRGRVTITVDTST | |
| | STAYMELSSLRSEDTAVYYCASSDPTGAWGQGTTVTVSS | |
| VH4-102G | QVQLVQSGAEVKKPGSSVKVSCKASGYTFTSYWIHWV | SEQ ID NO: 9 |
| | RQAPGQGLEWIGLIDPSDSYTNYNQKFRGRVTITVDTST | |
| | STAYMELSSLRSEDTAVYYCASSDPTGGWGQGTTVTVSS | |
| VH4-102L | QVQLVQSGAEVKKPGSSVKVSCKASGYTFTSYWIHWV | SEQ ID NO: 10 |
| | RQAPGQGLEWIGLIDPSDSYTNYNQKFRGRVTITVDTST | |
| | STAYMELSSLRSEDTAVYYCASSDPTGLWGQGTTVTVSS | |
| VH4-102S | QVQLVQSGAEVKKPGSSVKVSCKASGYTFTSYWIHWV | SEQ ID NO: 11 |
| | RQAPGQGLEWIGLIDPSDSYTNYNQKFRGRVTITVDTST | |
| | STAYMELSSLRSEDTAVYYCASSDPTGSWGQGTTVTVSS | |
| VH4-102T | QVQLVQSGAEVKKPGSSVKVSCKASGYTFTSYWIHWV | SEQ ID NO: 12 |
| | RQAPGQGLEWIGLIDPSDSYTNYNQKFRGRVTITVDTST | |
| | STAYMELSSLRSEDTAVYYCASSDPTGTWGQGTTVTVSS | |
| VH4-102V | QVQLVQSGAEVKKPGSSVKVSCKASGYTFTSYWIHWV | SEQ ID NO: 13 |
| | RQAPGQGLEWIGLIDPSDSYTNYNQKFRGRVTITVDTST | |
| | STAYMELSSLRSEDTAVYYCASSDPTGVWGQGTTVTVSS | |
| VH5-102C | QVQLVQSGAEVKKPGSSVKVSCKASGYTFTSYWIHWVR | SEQ ID NO: 14 |
| | QAPGQGLEWIGLIDPSDSYTNYNQKFRGRVTITRDTSTST | |
| | AYMELSSLRSEDTAVYYCASSDPTGCWGQGTTVTVSS | |
| VH5-102A | QVQLVQSGAEVKKPGSSVKVSCKASGYTFTSYWIHWVR | SEQ ID NO: 15 |
| | QAPGQGLEWIGLIDPSDSYTNYNQKFRGRVTITRDTSTST | |
| | AYMELSSLRSEDTAVYYCASSDPTGAWGQGTTVTVSS | |
| VH5-102G | QVQLVQSGAEVKKPGSSVKVSCKASGYTFTSYWIHWVR | SEQ ID NO: 16 |
| | QAPGQGLEWIGLIDPSDSYTNYNQKFRGRVTITRDTSTST | |
| | AYMELSSLRSEDTAVYYCASSDPTGGWGQGTTVTVSS | |
| VH5-102L | QVQLVQSGAEVKKPGSSVKVSCKASGYTFTSYWIHWVR | SEQ ID NO: 17 |
| | QAPGQGLEWIGLIDPSDSYTNYNQKFRGRVTITRDTSTST | |
| | AYMELSSLRSEDTAVYYCASSDPTGLWGQGTTVTVSS | |
| VH5-102S | QVQLVQSGAEVKKPGSSVKVSCKASGYTFTSYWIHWVR | SEQ ID NO: 18 |
| | QAPGQGLEWIGLIDPSDSYTNYNQKFRGRVTITRDTSTST | |
| | AYMELSSLRSEDTAVYYCASSDPTGSWGQGTTVTVSS | |
| VH5-102T | QVQLVQSGAEVKKPGSSVKVSCKASGYTFTSYWIHWVR | SEQ ID NO: 19 |
| | QAPGQGLEWIGLIDPSDSYTNYNQKFRGRVTITRDTSTST | |
| | AYMELSSLRSEDTAVYYCASSDPTGYWGQGTTVTVSS | |
| VH5-102V | QVQLVQSGAEVKKPGSSVKVSCKASGYTFTSYWIHWVR | SEQ ID NO: 20 |
| | QAPGQGLEWIGLIDPSDSYTNYNQKFRGRVTITRDTSTST | |
| | AYMELSSLRSEDTAVYYCASSDPTGVWGQGTTVTVSS | |
| VL (MEDR | DIVMTQAAFSNPVTPGTPASISCRSSKSLLHSSGITYLS | SEQ ID NO: 21 |
| 1230 Vκ2) | WYLQKPGQSPQLLIYQMSNLASGVPDRFSSSGSGTDF | |
| | TLKISRVEAEDVGVYYCAQNLELPLTFGQGTKLEIK | |
| Constant | ASTKGPSVFPLAPSSKSTSGGTAALGCLVKDYFPEPVTV | SEQ ID NO: 22 |
| Heavy Chain | SWNSGALTSGVHTFPAVLQSSGLYSLSSVVTVPSSSLGT | |
| (IgG1 Glm | QTYICNVNHKPSNTKVDKRVEPKSCDKTHTCPPCPAPE | |
| (3)) | LLGGPSVFLFPPKPKDTLMISRTPEVTCVVVDVSHEDPE | |
| | VKFNWYVDGVEVHNAKTKPREEQYNSTYRVVSVLTVL | |
| | HQDWLNGKEYKCKVSNKALPAPIEKTISKAKGQPREPQ | |
| | VYTLPPSREEMTKNQVSLTCLVKGFYPSDIAVEWESNG | |
| | QPENNYKTTPPVLDSDGSFFLYSKLTVDKSRWQQGNVF | |
| | SCSVMHEALHNHYTQKSLSLSPGK | |
| Constant light | RTVAAPSVFIFPPSDEQLKSGTASVVCLLNNFYPREAK | SEQ ID NO: 23 |
| chain | VQWKVDNALQSGNSQESVTEQDSKDSTYSLSSTLTLS | |
| | KADYEKHKVYACEVTHQGLSSPVTKSFNRGEC | |
| CDR-H3 102C | SDPTGC | SEQ ID NO: 24 |
| CDR-H3 102A | SDPTGA | SEQ ID NO: 25 |
| CDR-H3 102G | SDPTGG | SEQ ID NO: 26 |
| CDR-H3 102L | SDPTGL | SEQ ID NO: 27 |
| CDR-H3 102S | SDPTGS | SEQ ID NO: 28 |
| CDR-H3 102T | SDPTGT | SEQ ID NO: 29 |
| CDR-H3 102V | SDPTGV | SEQ ID NO: 30 |
| Human fibrin | YSMKKTTMKIIPFNRLTIG | SEQ ID NO: 31 |
| γ377-395 | | |
| VH4 102C- | CAAGTGCAGCTGGTGCAGTCGGGAGCCGAAGTCAAGAA | SEQ ID NO: 32 |
| DNA | GCCTGGTTCAAGCGTGAAAGTGTCCTGCAAGGCCTCCGG | |
| SEQUENCE | GTACACCTTCACGTCCTACTGGATTCACTGGGTCCGCCAA | |
| | GCACCAGGACAGGGCCTGGAGTGGATCGGACTGATCGAC | |
| | CCCAGCGACTCCTACACTAACTACAACCAGAAGTTCCGGG | |
| | GCAGAGTGACCATCACCGTGGATACCTCGACCAGCACTGC | |
| | TTATATGGAACTGTCGTCCCTCCGGTCCGAGGACACTGCC | |
| | GTGTACTACTGCGCGTCATCCGATCCGACTGGGTGTTGGG | |
| | GCCAGGGAACCACCGTGACAGTCTCCTCA | |
| VH5 102C- | CAAGTGCAGCTCGTGCAGTCCGGAGCCGAAGTCAAGAAG | SEQ ID NO: 33 |
| DNA | CCTGGCAGCTCAGTGAAAGTGTCCTGCAAGGCCTCGGGAT | |
| SEQUENCE | ACACCTTCACTTCCTACTGGATTCACTGGGTCAGACAAGC | |
| | GCCAGGACAGGGACTGGAGTGGATCGGGCTGATCGATCC | |
| | GTCCGACTCCTACACCAACTATAACCAGAAGTTCAGAGGC | |
| | CGCGTGACCATCACCAGGGACACTTCCACCTCGACGGCTT | |
| | ACATGGAGCTTAGCTCCCTGCGGTCGGAAGATACCGCCGT | |
| | GTACTACTGCGCAAGCTCAGACCCCACCGGTTGTTGGGGC | |
| | CAGGGCACTACTGTGACAGTCTCCTCA | |
| Chimeric 5B8 | CAAGTGCAGCTTCAGCAGCCGGGTGCTGA | SEQ ID NO: 34 |
| VH0 | ACTGGTCCGCCCTGGCACTTCCGTGAAGCT | |
| DNA | CAGCTGTAAAGCCTCCGGCTACACCTTCAC | |
| SEQUENCE | CTCATACTGGATTCACTGGGTCAAGCAAC | |
| | GGCCAGGACAAGGGTTGGAATGGATCGGG | |
| | CTCATCGATCCGTCCGATTCGTACACTAAC | |
| | TATAACCAGAAGTTCAGAGGAAAGGCCAC | |
| | CCTGACCGTGGACACCTCGTCGTCCACCGC | |
| | GTACATGCAGCTGTCAAGCCTGACTTCCG | |
| | AGGACTCCGCCGTGTACTACTGCGCAAGC | |
| | TCCGACCCCACTGGATGCTGGGGCCAGGG | |
| | TACCACGCTGACAGTCTCCTCA | |
| Chimeric 5B8 | QVQLQQPGAELVRPGTSVKLSCKASGYTFT | SEQ ID NO: 35 |
| VH0 | SYWIHWVKQRPGQGLEWIGLIDPSDSYTNY | |
| PROTEIN | NQKFRGKATLTVDTSSSTAYMQLSSLTSED | |
| SEQUENCE | SAVYYCASSDPTGCWGQGTTLTVSS | |
| Mouse | TFDSPYQVRRMRFSAQLLGLLVLWIPGSTADIVMTQAAFSNP | SEQ ID NO: 36 |
| Monoclonal | ITLGTSASMSCRSSKSLLHSSGITYLSWYLQKPGQSPQLLIYQ | |
| 5B8 Light | MSNLASGVPDRESSSGSGTDFTLRISRVEAEDVGVYYCAQNL | |
| Chain | ELPLTFGAGTKLELKRADAAPTVSACTKGEF | |
| Mouse | NTAFAGFGRNMRSLFSLQLLSTQDLAMGWSCIIVLLVSTATG | SEQ ID NO: 37 |
| Monoclonal | VHSQVQLQQPGAELVRPGTSVKLSCKASGYTFTSYWIHWVK | |
| 5B8 Heavy | QRPGQGLEWIGLIDPSDSYTNYNQKFRGKATLTVDTS | |
| Chain | SSTAYMQLSSLTSEDSAVYYCASSDPTGCWGQGTTLTVSPAS | |
| | TTPP | |

1. An isolated antibody that binds human fibrin or fibrinogen γC domain, comprising a heavy chain comprising a variable heavy (VH) chain sequence comprising three heavy chain CDR sequences, CDR-H1, CDR-H2, and CDR-H3, and a light chain comprising a variable light (VL) chain sequence comprising three light chain CDR sequences, CDR-L1, CDR-L2, and CDR-L3, wherein:
a. CDR-H1 comprises the sequence set forth in SEQ ID NO: 1,
b. CDR-H2 comprises the sequence set forth in SEQ ID NO: 2,
c. CDR-H3 comprises the sequence set forth in SEQ ID NO: 3, wherein X is Glycine (G), Valine (V), Threonine (T), Serine (S), Alanine (A) or Leucine (L).
d. CDR-L1 comprises the sequence set forth in SEQ ID NO: 4,
e. CDR-L2 comprises the sequence set forth in SEQ ID NO: 5, and
f. CDR-L3 comprises the sequence set forth in SEQ ID NO: 6.

2. The isolated antibody of claim 1, wherein the antibody comprises a VH sequence selected from a sequence set forth in one of SEQ ID NOs: 7-20.

3. The isolated antibody of claim 1 or 2, wherein the antibody comprises a VL sequence selected from a sequence set forth in SEQ ID NO 21.

4. The isolated antibody of any one of the above claims, wherein the antibody comprises a VH sequence selected from a sequence set forth in one of SEQ ID Nos: 7-20, and the VL sequence set for in SEQ ID NO: 21.

5. The isolated antibody of any one of the above claims, wherein the antibody comprises a VH sequence set forth in SEQ ID NO: 7 and a VL sequence set forth in SEQ ID NO: 21.

6. The isolated antibody of any one of the above claims, wherein the antibody comprises a VH sequence set forth in SEQ ID NO: 8 and a VL sequence set forth in SEQ ID NO: 21.

7. The isolated antibody of any one of the above claims, wherein the antibody comprises a VH sequence set forth in SEQ ID NO: 9 and a VL sequence set forth in SEQ ID NO: 21.

8. The isolated antibody of any one of the above claims, wherein the antibody comprises a VH sequence set forth in SEQ ID NO: 10 and a VL sequence set forth in SEQ ID NO: 21.

9. The isolated antibody of any one of the above claims, wherein the antibody comprises a VH sequence set forth in SEQ ID NO: 11 and a VL sequence set forth in SEQ ID NO: 21.

10. The isolated antibody of any one of the above claims, wherein the antibody comprises a VH sequence set forth in SEQ ID NO: 12 and a VL sequence set forth in SEQ ID NO: 21.

11. The isolated antibody of any one of the above claims, wherein the antibody comprises a VH sequence set forth in SEQ ID NO: 13 and a VL sequence set forth in SEQ ID NO: 21.

12. The isolated antibody of any one of the above claims, wherein the antibody comprises a VH sequence set forth in SEQ ID NO: 14 and a VL sequence set forth in SEQ ID NO: 21.

13. The isolated antibody of any one of the above claims, wherein the antibody comprises a VH sequence set forth in SEQ ID NO: 15 and a VL sequence set forth in SEQ ID NO: 21.

14. The isolated antibody of any one of the above claims, wherein the antibody comprises a VH sequence set forth in SEQ ID NO: 16 and a VL sequence set forth in SEQ ID NO: 21.

15. The isolated antibody of any one of the above claims, wherein the antibody comprises a VH sequence set forth in SEQ ID NO: 17 and a VL sequence set forth in SEQ ID NO: 21.

16. The isolated antibody of any one of the above claims, wherein the antibody comprises a VH sequence set forth in SEQ ID NO: 18 and a VL sequence set forth in SEQ ID NO: 21.

17. The isolated antibody of any one of the above claims, wherein the antibody comprises a VH sequence set forth in SEQ ID NO: 19 and a VL sequence set forth in SEQ ID NO: 21.

18. The isolated antibody of any one of the above claims, wherein the antibody comprises a VH sequence set forth in SEQ ID NO: 20 and a VL sequence set forth in SEQ ID NO: 21.

19. The isolated antibody of any one of the above claims, wherein the antibody is a humanized, human or chimeric antibody.

20. The isolated antibody of claim 19, wherein the antibody is a humanized antibody.

21. The isolated antibody of any one of the above claims, wherein the antibody comprises a heavy chain human constant region of a class selected from IgG, IgA, IgD, IgE, and IgM.

22. The isolated antibody of any one of the above claims, wherein the human Fc region comprises a human heavy chain constant region of the class IgG and a subclass selected from IgG1, IgG2, IgG3, and IgG4.

23. The isolated antibody of claim 22, wherein the human Fc region comprises wild-type, human IgG1 Fc.

24. The isolated antibody of claim 23, wherein the human Fc domain comprises the sequence set forth in SEQ ID NO: 22.

25. The isolated antibody of any one of the above claims, wherein the heavy chain comprises a constant heavy chain sequence set forth by SEQ ID NO: 22.

26. The isolated antibody of any one of the above claims, wherein the light chain comprises a constant light chain sequence set forth by SEQ ID NO: 23.

27. The isolated antibody of any one of the above claims, wherein the antibody comprises the VH sequence set forth in SEQ ID NO: 7, and the VL sequence set forth in SEQ ID NO: 21; and the human Fc region comprises wild-type, human IgG1 Fc.

28. The isolated antibody of any one of the above claims, wherein the antibody comprises the VH sequence set forth in SEQ ID NO: 8, and the VL sequence set forth in SEQ ID NO: 21; and the human Fc region comprises wild-type, human IgG1 Fc.

29. The isolated antibody of any one of the above claims, wherein the antibody comprises the VH sequence set forth in SEQ ID NO: 9, and the VL sequence set forth in SEQ ID NO: 21; and the human Fc region comprises wild-type, human IgG1 Fc.

30. The isolated antibody of any one of the above claims, wherein the antibody comprises the VH sequence set forth in SEQ ID NO: 10, and the VL sequence set forth in SEQ ID NO: 21; and the human Fc region comprises wild-type, human IgG1 Fc.

31. The isolated antibody of any one of the above claims, wherein the antibody comprises the VH sequence set forth in SEQ ID NO: 11, and the VL sequence set forth in SEQ ID NO: 21; and the human Fc region comprises wild-type, human IgG1 Fc.

32. The isolated antibody of any one of the above claims, wherein the antibody comprises the VH sequence set forth in SEQ ID NO: 12, and the VL sequence set forth in SEQ ID NO: 21; and the human Fc region comprises wild-type, human IgG1 Fc.

33. The isolated antibody of any one of the above claims, wherein the antibody comprises the VH sequence set forth in SEQ ID NO: 13, and the VL sequence set forth in SEQ ID NO: 21; and the human Fc region comprises wild-type, human IgG1 Fc.

34. The isolated antibody of any one of the above claims, wherein the antibody comprises the VH sequence set forth in SEQ ID NO: 14, and the VL sequence set forth in SEQ ID NO: 21; and the human Fc region comprises wild-type, human IgG1 Fc.

35. The isolated antibody of any one of the above claims, wherein the antibody comprises the VH sequence set forth in SEQ ID NO: 15, and the VL sequence set forth in SEQ ID NO: 21; and the human Fc region comprises wild-type, human IgG1 Fc.

36. The isolated antibody of any one of the above claims, wherein the antibody comprises the VH sequence set forth in SEQ ID NO: 16, and the VL sequence set forth in SEQ ID NO: 21; and the human Fc region comprises wild-type, human IgG1 Fc.

37. The isolated antibody of any one of the above claims, wherein the antibody comprises the VH sequence set forth in SEQ ID NO: 17, and the VL sequence set forth in SEQ ID NO: 21; and the human Fc region comprises wild-type, human IgG1 Fc.

38. The isolated antibody of any one of the above claims, wherein the antibody comprises the VH sequence set forth in SEQ ID NO: 18, and the VL sequence set forth in SEQ ID NO: 21; and the human Fc region comprises wild-type, human IgG1 Fc.

39. The isolated antibody of any one of the above claims, wherein the antibody comprises the VH sequence set forth in SEQ ID NO: 19, and the VL sequence set forth in SEQ ID NO: 21; and the human Fc region comprises wild-type, human IgG1 Fc.

40. The isolated antibody of any one of the above claims, wherein the antibody comprises the VH sequence set forth in SEQ ID NO: 20, and the VL sequence set forth in SEQ ID NO: 21; and the human Fc region comprises wild-type, human IgG1 Fc.

41. The isolated antibody of any one of claims 27-40, wherein the Fc region comprises one or more amino acid substitutions, wherein the one or more substitutions result in increased antibody half-life, increased ADCC activity, increased ADCP activity, or increased CDC activity compared with the Fc without the one or more substitutions.

42. The isolated antibody of any one of the above claims, wherein the Fc region binds an Fcγ Receptor selected from the group consisting of: FcγRI, FcγRIIa, FcγRIIb, FcγRIIc, FcγRIIIa, and FcγRIIIb.

43. The isolated antibody of any one of the above claims, wherein the antibody is a monoclonal antibody.

44. The antibody of claim 1, wherein the antibody binds an γ377-395 epitope of the fibrin or fibrinogen γC domain.

45. The isolated antibody of any one of the above claims, wherein the antibody binds to a peptide comprising SEQ ID NO: 31 with a K_{D} of less than or equal to about 1, 2, 3, 4, 5, 6, 7, or 8×10⁻⁵ M, as measured by surface plasmon resonance (SPR) single cycle kinetics (SCK) assay.

46. The isolated antibody of any one of the above claims, wherein the antibody binds to a peptide comprising the sequence of the γ377-395 epitope of the human fibrin or fibrinogen γC domain with a K_{D} of less than or equal to about 8×10-⁵ M, as measured by surface plasmon resonance (SPR) single cycle kinetics (SCK) assay.

47. The isolated antibody of any one of the above claims, wherein the antibody inhibits Mac-1 binding to fibrin or fibrinogen γC domain.

48. The isolated antibody of any one of the above claims, wherein the antibody exhibits inhibition of microglial adhesion to the fibrin or fibrinogen γC domain.

49. The isolated antibody of any one of the above claims for use in the treatment of a degenerative disorder of the nervous system.

50. An isolated polynucleotide or set of polynucleotides encoding the antibody of any of the above claims, a VH thereof, a VL thereof, a light chain thereof, a heavy chain thereof, or an antigen-binding portion thereof; optionally cDNA.

51. A vector or set of vectors comprising the polynucleotide or set of polynucleotides of claim 50.

52. A host cell comprising the polynucleotide or set of polynucleotides of claim 71 or the vector or set of vectors of claim 51.

53. A method of producing an antibody, the method comprising expressing the antibody with the host cell of claim 52 and isolating the expressed antibody.

54. A pharmaceutical composition comprising the antibody of any one of claims 1-46 and a pharmaceutically acceptable excipient.

55. A kit comprising the antibody of any one of claims 1-46 or a pharmaceutical composition of claim 54 and instructions for use

56. A method for treating a degenerative disorder of the nervous system, the method comprising administering to a mammalian subject a therapeutically effective amount the antibody of any one of claims 1-46 or a pharmaceutical composition of claim 54.

57. The method of claim 56, wherein the degenerative disorder of the nervous system is selected from the group consisting of: multiple sclerosis, spinal cord injury, stroke, and Alzheimer's Disease.

58. A method for treating a pathology associated with Mac-1 binding to fibrin or Mac-1 binding with fibrinogen, the method comprising administering to a mammalian subject a therapeutically effective amount the antibody of any one of claims 1-46 or a pharmaceutical composition of claim 54.

59. A method of inhibiting microglia activation, the method comprising administering to a mammalian subject a therapeutically effective amount the antibody of any one of claims 1-46 or a pharmaceutical composition of claim 54.

60. A method of preventing a degenerative disorder of the nervous system, the method comprising administering to a mammalian subject a therapeutically effective amount the antibody of any one of claims 1-46 or a pharmaceutical composition of claim 54.

61. A method of preventing colitis, the method comprising administering to a mammalian subject a therapeutically effective amount the antibody of any one of claims 1-46 or a pharmaceutical composition of claim 54.

62. A method of treating colitis, the method comprising administering to a mammalian subject a therapeutically effective amount the antibody of any one of claims 1-46 or a pharmaceutical composition of claim 54.
